(19) United States
(12) Patent Application Publication
(10) Pub. No.: US 2024/0189244 A1
(43) Pub. Date: Jun. 13, 2024

(54) COMPOSITIONS AND METHODS FOR DELIVERING PHARMACEUTICAL AGENTS

(71) Applicant: Arizona Board of Regents on Behalf of the University of Arizona, Tucson, AZ (US)

(72) Inventors: Heidi Mansour, Tucson, AZ (US); Rick Schnellmann, Tucson, AZ (US); David Encinas Basurto, Tucson, AZ (US); Ernest Vallorz, Tucson, AZ (US)

(21) Appl. No.: 18/285,971

(22) PCT Filed: Apr. 8, 2022
(86) PCT No.: PCT/US2022/023977

§ 371 Date: Oct. 6, 2023

Related U.S. Application Data
(60) Provisional application No. 63/172,941, filed on Apr. 9, 2021.

(51) Int. Cl. A61K 9/51; A61K 9/00; A61K 31/167; A61P 13/12

(52) U.S. Cl. CPC A61K 9/5153; A61K 9/0019; A61K 31/167; A61P 13/12

(57) ABSTRACT

Provided herein are compositions and methods for delivering pharmaceutical agents to the kidney. In particular, provided herein are nanoparticle formulations of β₂ adrenergic agonists for use in treating and preventing kidney damage or disease.

### CROSS-REFERENCE TO RELATED APPLICATION

The present application claims priority to U.S. Provisional Patent Application No. 63/172,941, filed Apr. 9, 2021, which is hereby incorporated by reference in its entirety.

### FIELD

Provided herein are compositions and methods for delivering pharmaceutical agents to the kidney. In particular, provided herein are nanoparticle formulations of β₂ adrenergic agonists for use in treating and preventing kidney damage or disease.

### BACKGROUND

Kidney failure occurs when they lose the ability to sufficiently filter waste from blood. Many factors can interfere with kidney health and function, such as exposure to toxic environmental pollutants or certain medications, and acute and chronic diseases such as diabetes, severe dehydration, and trauma.

Symptoms include reduced urine, swelling of legs, ankles, and feet from retention of fluids caused by the failure of the kidneys to eliminate water, unexplained shortness of breath, excessive drowsiness or fatigue, persistent nausea, confusion, pain or pressure in the chest, and seizures.

There are limited treatments for kidney failure including medications to control symptoms, dialysis, and kidney transplant.

Mitochondrial dysfunction is a primary consequence in both acute and chronic renal injury including drug and toxicant induced renal injury, ischemia-reperfusion injury and diabetic nephropathy (Feldkamp T, Kribben A, Weinberg J M. Assessment of mitochondrial membrane potential in proximal tubules after hypoxia-reoxygenation. Am J Physiol Renal Physiol. 2005; 288(6):F1092-102; Honda H M, Korge P, Weiss J N. Mitochondria and ischemia/reperfusion injury. Ann N Y Acad Sci. 2005; 1047:248-58; Cleveland K H, Brosius F C, Schnellmann R G. Regulation of mitochondrial dynamics and energetics in the diabetic renal proximal tubule by the β. Am J Physiol Renal Physiol. 2020; 319(5):F773-F9; Bellomo R, Kellum J A, Ronco C. Acute kidney injury. Lancet. 2012; 380(9843):756-66). In vitro mitochondrial dysfunction as a result of oxidant injury results in a decrease in the number and functionality of renal proximal tubule cell (RPTC) mitochondria as well as inhibition of ATP-dependent cellular repair processes (Rasbach K A, Schnellmann R G. Signaling of mitochondrial biogenesis following oxidant injury. J Biol Chem. 2007; 282(4):2355-62). This continual energy depletion and mitochondrial dysfunction hinders recovery and can lead to irreversible tissue and organ injury (Feldkamp et al., supra; Weinberg J M, Venkatachalam M A, Roeser N F, Nissim I. Mitochondrial dysfunction during hypoxia/reoxygenation and its correction by anaerobic metabolism of citric acid cycle intermediates. Proc Natl Acad Sci U S A. 2000; 97(6):2826-31; Coca S G, Yusuf B, Shlipak M G, Garg A X, Parikh C R. Long-term risk of mortality and other adverse outcomes after acute kidney injury: a systematic review and meta-analysis. Am J Kidney Dis. 2009; 53(6):961-73; Golestaneh L, Melamed M L, Hostetter T H. Uremic memory: the role of acute kidney injury in long-term outcomes. Kidney Int. 2009; 76(8):813-4).

As such, additional therapies for kidney disease and damage, in particular drug that can promote mitochondrial function and repair, are needed.

### SUMMARY

Agonism of the β₂-adrenergic receptor by formoterol results in mitochondrial biogenesis in the renal proximal tubules and can recover kidney function in a variety of injuries. However, intravenous administration of formoterol is limited by the potential for serious cardiovascular side effects. In experiments described herein, poly (ethylene glycol) methyl ether-block-poly(lactide-co-glycolide) nanoparticles containing formoterol were prepared by a modified single emulsion technique resulting in low opsonizing particles with a 442 nm median hydrodynamic diameter. These particles as well as formoterol were shown to be non-cytotoxic to tubular epithelial cells. Compared with intravenous administration of formoterol, the nanoparticle formulation of formoterol resulted in the complete attenuation of the tachycardic and hypotensive side effects of the free formoterol as well as decreased protein markers of mitochondrial biogenesis in the heart. The formoterol nanoparticles were shown to localize to the tubules of the renal cortex and improve the renal localization of formoterol compared to the free formoterol. The modified distribution of the nanoparticles containing formoterol allows for a significant mitochondrial biogenic or other efficacious effects in the kidneys at a fraction of the dose and minimize side effects at a lower dose. Accordingly, provided herein are compositions and methods for administering β₂ adrenergic agonists such as formoterol in nanoparticles for use in treating kidney damage and disease.

For example, in some embodiments, provided herein is a composition, comprising: a pharmaceutical composition comprising nanoparticles comprising a β₂ adrenergic agonist and a polymer (e.g., active agents dispersed within a polymer matrix). In some embodiments, the particles are spherical. In some embodiments, the nanoparticles have a diameter of 100 to 800 nm. In some embodiments, the beta 2 adrenergic agonist is released from the nanoparticle at physiological conditions. In some embodiments, each of the nanoparticles comprises 1 to 5 μg of said beta 2 adrenergic agonist per mg of nanoparticles. In some embodiments, the composition further comprises a pharmaceutically acceptable carrier.

The present disclosure is not limited to particular β₂ adrenergic agonists. Examples include but are not limited to, albuterol, formoterol, bitolterol, fenoterol, isoproterenol, levalbuterol, metaproterenol, pirbuterol, procaterol, ritodrine, terbutaline, arformoterol, bambuterol, clenbuterol, salmeterol, abediterol, carmoterol, indacaterol, olodaterol, vilanterol, isoxsuprine, mabuterol, or zilpaterol.

The present disclosure is not limited to particular polymers. In some embodiments, the polymer is biocompatible and biodegradable. Examples include but are not limited to, methyl ether-block-poly(lactide-co-glycolide) (PLGA), poly(vinyl alcohol), chitosan, poly(ε-caprolactone), poly(ethylene glycol) methyl ether-block-poly(lactide-co-glycolide) (PLGA-PEG), Poly(lactide-co-glycolide) methyl ether block-poly(ethylene glycol)-amine (PLGA-PEG-HN2), or poly(lactide-co-glycolide) methyl ether block-poly(ethylene glycol)-carboxylic acid (PLGA-PEG-COOH). In some embodiments, the PEG is amine modified, methyl end capped, and/or carboxy terminated. In some embodiments, the PLGA has an average molecular weight of 10,000 to 20,000 (e.g., 55,000) and the PEG has an average molecular weight of 2000 to 10,000 (e.g., 5,000).

The nanoparticles are made by any suitable method. One exemplary method comprises a) mixing a first solution comprising said β₂ adrenergic agonist with a second solution comprising an emulsion of said polymer in a solvent; b) emulsifying the first solution in the second solution; and c) removing the solvent. In other embodiments, the method comprises a) mixing a first solution comprising said β₂ adrenergic agonist with a second solution comprising an emulsion of the polymer in a solvent; b) emulsifying the first solution in the second solution to generate a first emulsion; c) mixing and emulsifying the first emulsion with a third solution comprising the polymer; and d) removing the solvent.

Additional embodiments provide a method of treating or preventing kidney disease, comprising: administering a composition described herein to a subject in need thereof. In some embodiments, the composition is administered to glomerular cells (e.g., podocytes, mesangial cells, endothelial cells). In some embodiments, the administering is intravenous administration, subcutaneous, or intraperitoneal. In some embodiments, the nanoparticles localize to the kidney of the subject (e.g., to the tubules of the renal cortex). In some embodiments, the administration does not result in cardiac side effects (e.g., tachycardic or hypotensive side effects). In some embodiments, the administration results in mitochondrial biogenesis in the renal proximal tubules.

The present disclosure is not limited to particular kidney diseases or damage. Examples include but are not limited to, acute renal injury, chronic renal injury, glomerular injury, drug and toxicant induced renal injury, ischemia-reperfusion injury, or diabetic nephropathy. In some embodiments, the subject has type II diabetes.

Further embodiments provide the use of a composition described herein to treat or prevent kidney disease in a subject or a composition described herein for use in treating or preventing kidney disease in a subject.

Additional embodiments are described herein.

### DESCRIPTION OF THE FIGURES

FIG. 1. Nanoparticles (NP) containing formoterol were prepared as described. (A) NP hydrodynamic diameter was measured via dynamic light scattering (DLS) using the Malvern Zetasizer Nano ZS. Size distribution is representative (n=3). (B) NP size and morphology was assessed via SEM. (C) Zeta potential was determined at a concentration of 0.1 mg/mL of NP suspended in 0.1× normal saline at 25° C. and a measured pH of 7.2. Zeta potential is representative (n=3).

FIG. 2. Cumulative formoterol release from nanoparticle suspension over time. Nanoparticles containing formoterol are dispersed in 1x PBS with stirring at 37° C. Data are presented as mean+s.d. (n=3)

FIG. 3. (A) Formoterol and (B) PLGA-PEG Nanoparticles (NP) do not impact viability of renal proximal tubule cells (RPTCs). Renal proximal tubule cells isolated from New Zealand white rabbits were plated at a density of 18,000 cells/well. After reaching confluence, cells were exposed to either formoterol or PLGA-PEG NP at concentrations from 1 to 1000 nM (formoterol) or 0.001 to 1 mg/mL (NP) for 24 hours. After 24 hours of exposure, cells were washed with fresh media and viability was assessed by resazurin assay. Data were analyzed via one-way ANOVA followed by Dunnett's post hoc test to evaluate differences between groups. Data are presented as the mean of+s.d. (n=3 biological replicates). ns, no significant difference versus vehicle control.

FIG. 4. Evaluation of NP renal localization. Renal cross-sections are co-stained with anti-polyethylene glycol (PEG) antibody to visualize NP (red), phytohemagglutinin (PHA) lectin to identify renal proximal tubules (green) and counterstained with 40,6-diamidino-2-phenylindole (DAPI) to visualize cell nuclei (blue). C57BL/6 mice exposed to (A) NP2 (0.04 mg/kg) or (B) normal saline. Images are representative of n=3 animals per group. (C) Formoterol concentration in renal cortex as determined by LCMS-MS at 3 and 24-hours post administration. Data were analyzed via student's t-test to evaluate differences between groups. Data are presented as the mean+s.d. (n=4-5). * indicates significant difference between groups.

FIG. 5. Nanoparticles containing formoterol stimulate mitochondrial biogenesis. C57Bl/6 mice received a single lateral tail vein injection of either vehicle, formoterol (0.3 mg/kg), or formoterol containing nanoparticle (0.04 mg/kg formoterol). Renal cortex (A-C) and left ventricle (D-F) was evaluated by immunoblotting for PGC1a (A&D), ND1 (B&E) and NDUFS1 (C&F) expression and quantified via densitometry. (G) Representative immunoblots. Data were analyzed via one-way ANOVA followed by Dunnett's post hoc test to evaluate differences between groups. Data are presented as the mean of+s.e.m. (n=8-9). *P, 0.05 versus vehicle control.

FIG. 6. Representative electron micrographs of renal cortical sections from mice with either exposed to either (A) saline, (B) 0.3 mg/kg formoterol, (C) 0.04 mg/kg formoterol NP. (D) Quantification of mitochondria per field (E) Quantification of individual mitochondrial area. Data were analyzed via one-way ANOVA followed by Dunnett's post hoc test to evaluate differences between groups. Data are presented as the mean of+s.e.m. (n=5). *P, 0.05 versus vehicle control.

FIG. 7. Naïve C57BL/6 mice were dosed with either saline, formoterol containing nanoparticles (0.04 mg/kg) or formoterol free drug (0.3 mg/kg). Animal heart rate and blood pressure was measured between 1-2 hours following treatment. Data show a significant (*) increase in (A) heart rate and decrease in (B) mean arterial pressure (MAP) and (C) diastolic blood pressure for the formoterol treatment group but not for the formoterol nanoparticle group when compared to saline control. Data were analyzed via one-way ANOVA followed by Dunnett's post hoc test to evaluate differences between groups. Data are presented as the mean of+s.d. *=p<0.05

FIG. 8. Solubility of formoterol determined in (A) ethanol and methanol (B) water at various pH and (C) aqueous solutions of various surfactants at 25° C. and gentle shaking for 24 hours. Data presented are mean (n=3)+s.d.

FIG. 9. Formoterol drug loading of nanoparticles (A) prepared by single emulsion with increasing PLGA molecular weight (B) prepared by single emulsion with increasing PLGA concentration (C) prepared by double emulsion with modified PEG groups (D) prepared by double emulsion with alterations to inner aqueous phase surfactant (E) structures of sodium cholate and sodium deoxycholate. PVA; 1% polyvinyl alcohol, NaCho; 12 mM sodium cholate, NaDOChol; 10 mM sodium deoxycholate. * indicates significance (p<0.05). All data are presented as mean (n=3)+s.d.

FIG. 10. Median hydrodynamic diameter determined by zetasizer for nanoparticles prepared by double emulsion with (A) increasing 60 W ultrasonication time and (B) increasing polymer concentration in the organic phase. All data are presented as mean (n=3)+s.d.

FIG. 11. Median hydrodynamic diameter determined by zetasizer for PLGA-PEG-NH₂ nanoparticles prepared by double emulsion using either ▴ mannitol, ▪ trehalose or ● sucrose as cryoprotectants. All data are presented as mean (n=3)±s.d.

FIG. 12. Zeta potential measurements of nanoparticles prepared with various PEG modifications immediately following synthesis after resuspension in 0.1× normal saline solution at 25° C. and pH 7.2. [symbol] PLGA-PEG-COOH, [symbol] PLGA-PEG, [symbol] PLGA-PEG-NH₂. All data are presented as mean (n=3)±s.d.

FIG. 13. Cumulative formoterol release from nanoparticles at 37° C. and constant stirring (A) [symbol] oil-in-water single emulsion [symbol] water-in-oil-in-water double emulsion (B) nanoparticles prepared by double emulsion with [symbol] 1% PVA inner phase, [symbol] 12 mM sodium cholate inner phase, [symbol] 10 mM sodium deoxycholate inner phase (C) nanoparticles prepared by double emulsion using [symbol] 10 mg/mL, [symbol] 20 mg/mL, [symbol] 50 mg/mL PLGA-PEG-NH₂. Inserts of the first 10 hours are included for (B) and (C). All data are presented as the mean of (n=3)±s.d.

FIG. 14. Scanning electron micrographs taken of double emulsion prepared nanoparticles of (A) PLGA-PEG, (B) PLGA-PEG-COOH, (C) PLGA-PEG-NH2.

FIG. 15. XRPD diffractograms (A) formoterol fumarate dihydrate (orange), PLGA-PEG-NH2 (red), sucrose (green), and trehalose (blue) and (B) nanoparticles lyophilized without cryoprotectant (red), nanoparticles lyophilized with either sucrose (green) or trehalose (blue) cryoprotectants. Black is the blank background.

FIG. 16. Differential scanning calorimetry thermograms of (A) formoterol fumarate dihydrate, (B) PLGA-PEG-NH2 (C) Sucrose, (D) Trehalose, (E) PLGA-PEG-NH2 nanoparticles lyophilized without cryoprotectant, (F) nanoparticles lyophilized with 5% sucrose cryoprotectant, (G) nanoparticles lyophilized with 5% trehalose cryoprotectant.

FIG. 17. Representative hot stage microscopy images for (A) PLGA-PEG-NH2 nanoparticles lyophilized without cryoprotectant, (B) nanoparticles lyophilized with 5% sucrose cryoprotectant, (C) nanoparticles lyophilized with 5% trehalose cryoprotectant.

FIG. 18. (a) Mice subjected to I/R injury or sham were administered DEDC-NP 24 hours after injury and organs extracted and imaged 1 hour after injection (b) fluorescence intensities from each of the evaluated organs (c) fluorescence intensity of each organ normalized to individual cardiac intensity (n=5)

FIG. 19. (a) Mice subjected to I/R injury or sham were administered formoterol or vehicle therapy daily starting 24 hours post injury. (b) Mice subjected to I/R injury or sham were administered formoterol or vehicle therapy at 24 hours post injury and again 96 hours post injury. (c) Serum KIM-1 and (d) serum NGAL for mice at 24, 96 and 144 hours post injury. VS—Vehicle saline, VNP—Vehicle nanoparticle, FFD—Formoterol free drug, FNP—Formoterol containing nanoparticles (n=5-6)

FIG. 20. Renal cortex from mice subjected to I/R injury 144 hours post injury and stained with anti-KIM-1 antibody. Mice were treated daily with either (a) VS (b) VNP (c) FFD or (d) FNP. Renal cortex from sham (e). Quantitative analysis of renal KIM-1 expression from mice 144 hours post surgery (f) and representative western blots (g). I/R—Ischemia-Reperfusion.

FIG. 21. Renal cortex from mice subjected to I/R injury 144 hours post injury, quantitative analysis of renal PGC-1α (a) and representative western blots (b). (n=5-6)

FIG. 22. Renal cortex from mice subjected to I/R injury 144 hours post injury, quantitative analysis of renal mitochondrial proteins (a) ATP5A, (b) UQCRC2, (c) SDHB, (d) NDUFB8, and representative western blots (e). (n=5)

FIG. 23. Electron micrographs showing mitochondria (arrows) of mouse proximal tubules 144 hours post surgery. Mice were treated with either (a, b) VS (c, f) VNP (d, g) FFD or (e, h) FNP. Analysis of mitochondrial number per field (i) and total mitochondrial area per field (j). Scale bars are 2 μm. (n=5)

FIG. 24. Renal cortex from mice subjected to I/R injury 144 hours post injury, quantitative analysis of renal tight junction proteins (a) ZO-1, (b) Occludin, (c) Claudin-5, and representative western blots (d). (n=5)

FIG. 25. PAS stained microscopy sections of renal cortex 144 hours post surgery. Mice were treated with either (a, b) VS (c, f) VNP (d, g) FFD or (e, h) FNP. Analysis of (i) degree of tubular necrosis (j) degree of protein cast formation per field. (n=5)

FIG. 26. Trichrome stained microscopy sections of the renal cortex 144 hours post surgery. Mice were treated with either (a, b) VS, (c, f) VNP, (d, g) FFD, or (e, h) FNP. (i) analysis of the % of collagen per histological field. scale bar is 150 μm, (n=5)

FIG. 27. (a) Heart rate measurements taken from mice 1-2 hours following administration of either formoterol or vehicle therapy. (b) Mean arterial pressure measurements taken from mice 1-2 hours following administration of either formoterol or vehicle therapy. (c) Diastolic blood pressure measurements taken from mice 1-2 hours following administration of either formoterol or vehicle therapy. (n=5)

FIG. 28. Left ventricle from mice subjected to I/R injury 144 hours post injury, quantitative analysis of (a) ATP5A, (b) UQCRC2, (c) SDHB, (d) NDUFB8, (e) MHC, (f) ANP. The total cardiac mass (g) from I/R injured mice and (h) representative western blots. (n=5)

FIG. 29. Hydrodynamic particle size distribution analysis of (a) FNP and (b) VNP nanoparticles. VNP—Vehicle nanoparticle, FNP—Formoterol containing nanoparticles Representative of (n=3) measurements.

FIG. 30. Quantitative analysis of renal KIM-1 expression from mice 144 hours post surgery amongst sham mice treated with VS, VNP, FFD, and FNP. (n=5)

FIG. 31. Electron micrographs of mouse proximal tubules 144 hours post I/R injury treated with either VS, VNP, FFD, FNP were analyzed for individual mitochondrial area. (n=5)

FIG. 32. Measurements taken from naïve mice 1-2 hours following administration of either VS, VNP, FFD, or FNP. (a) heart rate, (b) mean arterial pressure, (c) diastolic blood pressure. (n=5)

FIG. 33.—Nanoparticle characterization of DEDC(NP) (a) hydrodynamic size distribution and (b) zeta potential. Representative FFD(NP) (c) hydrodynamic size distribution and (d) zeta potential (e) cumulative formoterol released from FFD(NP) at 37° C. in PBS from 0-144 h.

FIG. 34. Nanoparticle biocompatibility and uptake in RPTCs. (a) DEDC(NP)s have no impact on the viability of RPTCs up to 1 mg/mL after 24 hours (b) representative fluorescence microscopy of DEDC and DEDC(NP)s uptaken by RPTCs 2 h after exposure (c) DEDC(NP)s have significantly increased fluorescent intensity compared to DEDC dye in media alone (d) representative fluorescence microscopy of DEDC(NP)s uptaken by RPTCs pretreated with endocytosis inhibitors (e) quantitative uptake of nanoparticles by RPTCs with inhibitors chlorpromazine, nocodazole, and simvastatin. [symbol] chlorpromazine, [symbol] nocodazole [symbol] simvastatin. RPTC—renal proximal tubule cells, ns—not significant, all n=5 biological replicates.

FIG. 35.—Kidney specific accumulation of nanoparticles. (a) representative whole body fluorescence imaging of mice 3 h following administration of 30 mg/kg DEDC(NP) or equivalent amount of free DEDC (b) fluorescence intensity in the kidney region of interest of mice following administration of 30 mg/kg DEDC(NP) or equivalent amount of free DEDC (c) representative ex vivo fluorescence of individual organs 3 h following administration of 30 mg/kg DEDC(NP) (d) individual organ ex vivo fluorescence intensity normalized to organ weight (e) individual organ ex vivo fluorescence intensity normalized to cardiac fluorescence. Cont. [symbol] —DEDC free dye control, i.v. [symbol] —intravenous, i.p. [symbol] —intraperitoneal, s.c. [symbol] —subcutaneous, p.o. [symbol] —per os. (n=4-5)

FIG. 36. Whole body fluorescence of nanoparticles up to 144 h post administration. left to right in each image are DEDC free dye control, intravenous, intraperitoneal, subcutaneous, per os (n=5)

FIG. 37. Mouse weight gain following NP dosing for 1 week with either Veh(Sal), Veh(NP), FFD(NP) or FFD(Sal) (n=6)

FIG. 38. Electron transport chain protein expression following 1 week of FFD(NP), FFD(Sal), Veh(Sal) or Veh(NP) administration (a) NDUFB8 (complex I) expression (b) SDHB (complex II) expression (c) UQCRC2 (complex III) expression (d) ATP5A (complex V) expression (e) representative western blots. Animals were administered FFD(NP) and Veh(NP) either once or twice per week or FFD(Sal) or Veh(Sal) daily (n=6),

FIG. 39. Initial body weight of WT and ob/ob mice.

FIG. 40. Initial blood glucose of WT and ob/ob mice

FIG. 41. Body weight and blood glucose of mice. WT and BTBR ob/ob mice were administered Veh(Sal), Veh(NP), FFD(Sal) or FFD(NP) therapy for eight weeks starting at 5 weeks of age and ending after 12 weeks of age (a & b) body weight was measured weekly and (c & d) blood glucose was measured at 5, 8 and 12 weeks of age. [symbol] WT Veh(Sal), [symbol] WT Veh(NP), [symbol] WT FFD(Sal), [symbol] WT FFD(NP), [symbol] ob/ob Veh(Sal), [symbol] ob/ob Veh(NP), [symbol] ob/ob FFD(Sal), [symbol] ob/ob FFDN(NP)

FIG. 42. Initial ACR of WT and ob/ob mice taken from the initial vehicle treated cohorts.

FIG. 43. Urinalysis of WT and ob/ob mice. WT and BTBR ob/ob mice were administered Veh(Sal), Veh(NP), FFD(Sal) or FFD(NP) therapy for eight weeks starting at 5 weeks of age and ending after 12 weeks of age (a & b) urine volume (c & d) urinary albumin creatinine ratio (e & f) transdermal estimated glomerular filtration rate collected between 5 and 12 weeks of age (g) 12-week urinary KIM-1 (h) 12-week urinary NGAL. [symbol] WT Veh(Sal), [symbol] WT Veh(NP), [symbol] WT FFD(Sal), [symbol] WT FFD(NP), [symbol] ob/ob Veh(Sal), [symbol] ob/ob Veh(NP), [symbol] ob/ob FFD(Sal), [symbol] ob/ob FFDN(NP)

FIG. 44. Histopathology of glomerular injury in WT and ob/ob mice. WT and BTBR ob/ob mice were administered Veh(Sal), Veh(NP), FFD(Sal) or FFD(NP) therapy for eight weeks starting at 5 weeks of age and ending after 12 weeks of age (a) representative PAS-stained glomeruli of 12-week-old WT and ob/ob mice. Scale bar is 100 μm (b) mesangial matrix as a fraction of the glomerular tuft in 12-week-old mice (c) glomerular tuft size in 12-week-old mice (d) representative picrosirius red-stained glomeruli of 12-week-old WT and ob/ob mice. Scale bar is 50 μm (e) quantification of the picrosirius red-positive area as a fraction of the glomerular tuft.

FIG. 45. Histopathology of tubular injury in WT and ob/ob mice. WT and BTBR ob/ob mice were administered Veh(Sal), Veh(NP), FFD(Sal) or FFD(NP) therapy for eight weeks starting at 5 weeks of age and ending after 12 weeks of age (a) representative F4/80 IHC stained sections of renal cortex of 12-week-old WT and ob/ob mice. Scale bar is 100 μm (b) number of F4/80 positive cells per high powered field (c) representative picrosirius red-stained sections of renal cortex of 12-week-old WT and ob/ob mice. Scale bar is 100 μm (d) quantification of the picrosirius red-positive area as a fraction of the glomerular-free field.

FIG. 46. Profibrotic protein expression in the renal cortex in WT and BTBR ob/ob mice that were administered Veh(Sal), Veh(NP), FFD(Sal) or FFD(NP) therapy for eight weeks starting at 5 weeks of age and ending after 12 weeks of age (a) Collagen I expression (b) pSMAD3/SMAD3 expression (c) TGF-β1 expression (d) representative western blots.

FIG. 47. Myocardial fibrosis in mice exposed to either (a) Veh(Sal) (b) FFD(Sal) or (c) FFD(NP) for 8 weeks. Solid arrows indicate interstitial fibrosis, dashed arrows indicate replacement fibrosis.

FIG. 48. Formoterol containing nanoparticles protect against cardiovascular damage in WT and BTBR ob/ob mice that were administered Veh(Sal), Veh(NP), FFD(Sal) or FFD(NP) therapy for eight weeks starting at 5 weeks of age and ending after 12 weeks of age (a) representative Masson's trichrome-stained interstitial and perivascular sections from the left ventricular wall of 12-week-old mice. Scale bar is 50 μm (b) quantitative analysis of interstitial trichrome staining intensity (c) quantitative analysis of perivascular trichrome staining intensity (histology Veh(Sal) and FFD(NP) n=8, FFD(Sal) n=4) (d) heart weights of WT and ob/ob mice (e) heart/body weight normalized to respective WT or ob/ob Veh(sal) treated mice (FFD(Sal) mice are between 10-12 weeks of age all others are 12 weeks of age, FFD(Sal) n=4-7, all other groups n=8) (f) survival probability for treatment groups Veh(Sal) & Veh(NP)(black), FFD(Sal)(red), FFD(NP)(green), dashed line is WT, solid line is ob/ob (P<0.05)

### DEFINITIONS

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human or non-human mammal subject.

As used herein, the term “diagnosed,” as used herein, refers to the recognition of a disease by its signs and symptoms (e.g., resistance to conventional therapies), or genetic analysis, pathological analysis, histological analysis, and the like.

As used herein, the term “effective amount” refers to the amount of an agent (e.g., a composition of the present disclosure) sufficient to effect beneficial or desired results. An effective amount can be administered in one or more administrations, applications or dosages and is not limited to a particular formulation or administration route.

As used herein, the term “co-administration” refers to the administration of at least two agent(s) (e.g., a composition of the present disclosure) and one or more additional agents or therapies to a subject. In some embodiments, the co-administration of two or more agents/therapies is concurrent. In some embodiments, a first agent/therapy is administered prior to a second agent/therapy. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

As used herein, the term “pharmaceutical composition” refers to the combination of an active agent with a carrier, inert or active, making the composition especially suitable for diagnostic or therapeutic use in vivo, in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants. (See e.g., Martin, Remington's Pharmaceutical Sciences, 15th Ed., Mack Publ. Co., Easton, PA, (1975)).

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water and industrial samples. Such examples are not however to be construed as limiting the sample types applicable to the present disclosure.

As used herein, the terms “purified” or “to purify.” refer, to the removal of undesired components from a sample. As used herein, the term “substantially purified” refers to molecules that are at least 60% free, at least 65% free, at least 70% free, at least 75% free, at least 80% free, at least 85% free, at least 90% free, at least 95% free, at least 96% free, at least 97% free, at least 98% free, at least 99% free, or 100% free from other components with which they usually associated.

As used herein, the term “modulate” refers to the activity of a compound (e.g., a compound of the present disclosure) to affect (e.g., to promote or retard) an aspect of cellular function.

As used herein, the phrase “in need thereof” means that the subject has been identified as having a need for the particular method or treatment. In some embodiments, the identification can be by any means of diagnosis. In any of the methods and treatments described herein, the subject can be in need thereof. In some embodiments, the subject is in an environment or will be traveling to an environment in which a particular disease, disorder, condition, or injury is prevalent.

### DETAILED DESCRIPTION

Mitochondrial biogenesis has been shown to promote recovery of mitochondrial and cellular function (Rasbach K A, Schnellmann R G. Signaling of mitochondrial biogenesis following oxidant injury. J Biol Chem. 2007; 282(4):2355-62; Rasbach K A, Schnellmann R G. PGC-1alpha over-expression promotes recovery from mitochondrial dysfunction and cell injury. Biochem Biophys Res Commun. 2007; 355(3):734-9; Bhargava P, Janda J, Schnellmann R G. Elucidation of cGMP-dependent induction of mitochondrial biogenesis through PKG and p38 MAPK in the kidney. Am J Physiol Renal Physiol. 2020; 318(2):F322-F8; Dupre T V, Jenkins D P, Muise-Helmericks R C, Schnellmann R G. The 5-hydroxytryptamine receptor 1F stimulates mitochondrial biogenesis and angiogenesis in endothelial cells. Biochem Pharmacol. 2019; 169:113644) via induction of the peroxisome proliferator-activated receptor-γ coactivator-1α (PGC-1α) which is expressed in highly metabolic tissues such as the heart, skeletal muscle and kidneys (Scarpulla R C. Transcriptional paradigms in mammalian mitochondrial biogenesis and function. Physiol Rev. 2008; 88(2):611-38). Overexpression of PGC-1α following treatment of RPTCs in vitro showed increased mitochondrial DNA copy number as well as increased expression of electron transport chain (ETC) proteins and elevated mitochondrial respiration rate (Funk J A, Odejinmi S, Schnellmann R G. SRT1720 induces mitochondrial biogenesis and rescues mitochondrial function after oxidant injury in renal proximal tubule cells. J Pharmacol Exp Ther. 2010; 333(2):593-601).

The Food and Drug Administration-approved β₂-adrenergic receptor agonist formoterol is a potent inducer of PGC-1α and mitochondrial biogenesis in RPTCs (Wills L P, Trager R E, Beeson G C, Lindsey C C, Peterson Y K, Beeson C C, et al. The β2-adrenoceptor agonist formoterol stimulates mitochondrial biogenesis. J Pharmacol Exp Ther. 2012; 342(1):106-18; Cameron R B, Peterson Y K, Beeson C C, Schnellmann R G. Structural and pharmacological basis for the induction of mitochondrial biogenesis by formoterol but not clenbuterol. Sci Rep. 2017; 7(1):10578). Additionally, formoterol treatment has been shown to improve renal function in vivo via mitochondrial biogenesis in mouse models of acute kidney injury (Wills et al, supra; Jesinkey S R, Funk J A, Stallons L J, Wills L P, Megyesi J K, Beeson C C, et al. Formoterol restores mitochondrial and renal function after ischemia-reperfusion injury. J Am Soc Nephrol. 2014; 25(6):1157-62; Cameron R B, Gibbs W S, Miller S R, Dupre T V, Megyesi J, Beeson C C, et al. Proximal Tubule. J Pharmacol Exp Ther. 2019; 369(1):173-80) as well as maintaining mitochondrial dynamics in an early model of diabetic kidney disease (Cleveland et al., supra).

While the β₂-adrenergic receptor is ubiquitously expressed, its significantly increased renal proximal tubule expression, compared to other renal structures, makes the tubules the primary target for therapeutic effect (Cameron et al., supra). The prevalence of β₂-adrenergic receptors outside of the kidneys supports that systemic formoterol exposure will have many off-target effects, which may result in significant toxicity. Cardiac specific PGC-1α overexpression leads to increases in mitochondrial number in cardiomyocytes which is linked with edema and dilated cardiomyopathy (Villena J A. New insights into PGC-1 coactivators: redefining their role in the regulation of mitochondrial function and beyond. FEBS J. 2015; 282(4):647-72; Lehman J J, Barger P M, Kovacs A, Saffitz J E, Medeiros D M, Kelly D P. Peroxisome proliferator-activated receptor gamma coactivator-1 promotes cardiac mitochondrial biogenesis. J Clin Invest. 2000; 106(7):847-56) and compounds that similarly induce mitochondrial biogenesis for the treatment of chronic kidney disease and type 2 diabetes showed an increase in the rate of heart failure events in clinical trials (de Zeeuw D, Akizawa T, Audhya P, Bakris G L, Chin M, Christ-Schmidt H, et al. Bardoxolone methyl in type 2 diabetes and stage 4 chronic kidney disease. N Engl J Med. 2013; 369(26):2492-503). Formoterol has also been shown to induce mitochondrial biogenesis in adult feline cardiomyocytes at a concentration one third of that required for mitochondrial biogenesis in RPTCs (Wills et al., supra).

Apart from long-term effects of mitochondrial biogenesis on heart, immediate chronotropic and ionotropic effects of intravenous administration of formoterol are also of concern (Levine M A, Leenen F H. Role of beta 1-receptors and vagal tone in cardiac inotropic and chronotropic responses to a beta 2-agonist in humans. Circulation. 1989; 79(1):107-15; Brodde O E. Beta 1- and beta 2-adrenoceptors in the human heart: properties, function, and alterations in chronic heart failure. Pharmacol Rev. 1991; 43(2):203-42). Systemic formoterol administration in Wistar rats showed an increase in heart rate of 100-150 bpm from baseline immediately following dosing, which persisted for up to 4 hours. This effect was seen with formoterol doses as low as 0.003 mg/kg which is significantly less than the 0.3 mg/kg effective dose required for mitochondrial biogenesis in the above studies (Koziczak-Holbro M, Rigel D F, Dumotier B, Sykes D A, Tsao J, Nguyen N H, et al. Pharmacological Characterization of a Novel 5-Hydroxybenzothiazolone-Derived. J Pharmacol Exp Ther. 2019; 369(2):188-99). Additionally, intravenous formoterol administration resulted in a significant decrease in mean arterial pressure (MAP) at similar doses (Koziczak-Holbro M, Rigel D F, Dumotier B, Sykes D A, Tsao J, Nguyen N H, et al. Pharmacological Characterization of a Novel 5-Hydroxybenzothiazolone-Derived. J Pharmacol Exp Ther. 2019; 369(2):188-99). As a result, translation of formoterol to the clinic is severely limited by the current formulation.

The present disclosure addresses these concerns by encapsulation of formoterol within a polymeric nanoparticle carrier. These carriers offer the ability to encapsulate a wide variety of molecules including nucleic acids, proteins and peptides as well as small molecules and otherwise insoluble drugs (Makadia H K, Siegel S J. Poly Lactic-co-Glycolic Acid (PLGA) as Biodegradable Controlled Drug Delivery Carrier. Polymers (Basel). 2011; 3(3):1377-97). An additional benefit is the ability of nanoparticle delivery systems to modify the pharmacokinetics of loaded compounds especially with regard to the kidneys where many drugs, including formoterol, are cleared however they persist in the kidneys for too brief a window to have the desired therapeutic effect (Duncan R, Gaspar R. Nanomedicine(s) under the microscope. Mol Pharm. 2011; 8(6):2101-41; Singh R, Lillard J W. Nanoparticle-based targeted drug delivery. Exp Mol Pathol. 2009; 86(3):215-23; Sasaki H, Kamimura H, Shiobara Y, Esumi Y, Takaichi M, Yokoshima T. Disposition and metabolism of formoterol fumarate, a new bronchodilator, in rats and dogs. Xenobiotica. 1982; 12(12):803-12; Labhasetwar V, Song C, Humphrey W, Shebuski R, Levy R J. Arterial uptake of biodegradable nanoparticles: effect of surface modifications. J Pharm Sci. 1998; 87(10):1229-34). Targeting of nanoparticles to the kidneys additionally allows for a reduction of undesired off target effects as well as a reduction in the overall administered dose as a greater proportion of the drug is available at the target site (Wang A Z, Langer R, Farokhzad O C. Nanoparticle delivery of cancer drugs. Annu Rev Med. 2012; 63:185-98; Song C X, Labhasetwar V, Murphy H, Qu X, Humphry W R, Shebuski R J, et al. Formulation and characterization of biodegradable nanoparticles for intravascular local drug delivery Journal of Controlled Release 1997. p. 197-212; Westedt U, Kalinowski M, Wittmar M, Merdan T, Unger F, Fuchs J, et al. Poly(vinyl alcohol)-graft-poly(lactide-co-glycolide) nanoparticles for local delivery of paclitaxel for restenosis treatment. J Control Release. 2007; 119(1):41-51). This has been exploited for a variety of kidney diseases however there are currently no clinically approved therapeutic nanoparticles and few preclinical research studies have successfully targeted the renal tubule specifically (Williams R M, Jaimes E A, Heller D A. Nanomedicines for kidney diseases. Kidney Int. 2016; 90(4):740-5; Nair A V, Keliher E J, Core A B, Brown D, Weissleder R. Characterizing the interactions of organic nanoparticles with renal epithelial cells in vivo. ACS Nano. 2015; 9(4):3641-53; Williams R M, Shah J, Ng B D, Minton D R, Gudas L J, Park C Y, et al. Mesoscale nanoparticles selectively target the renal proximal tubule epithelium. Nano Lett. 2015; 15(4):2358-64; Williams R M, Shah J, Tian H S, Chen X, Geissmann F, Jaimes E A, et al. Selective Nanoparticle Targeting of the Renal Tubules. Hypertension. 2018; 71(1):87-94; Han S J, Williams R M, D'Agati V, Jaimes E A, Heller D A, Lee H T. Selective nanoparticle-mediated targeting of renal tubular Toll-like receptor 9 attenuates ischemic acute kidney injury. Kidney Int. 2020; 98(1):76-87; Yu H, Lin T, Chen W, Cao W, Zhang C, Wang T, et al. Size and temporal-dependent efficacy of oltipraz-loaded PLGA nanoparticles for treatment of acute kidney injury and fibrosis. Biomaterials. 2019; 219:119368).

The present disclosure provides a polymeric nanoparticle drug delivery system that safely induces mitochondrial biogenesis in the proximal tubule cells of kidneys while reducing toxic side effects.

Accordingly, provided herein are nanoparticle formulations of β₂ adrenergic agonists (e.g., formoterol) that allow for delivery to the kidneys without cardiac toxicity.

For example, in some embodiments, provided herein is a composition, comprising: a pharmaceutical composition comprising nanoparticles comprising a β₂ adrenergic agonist and a polymer (e.g., active agents encapsulated by a polymer shell).

In some embodiments, the particles are spherical. In some embodiments, the nanoparticles have a diameter of 100 to 800 nm. In some embodiments, the β₂ adrenergic agonist is released from the nanoparticle at physiological conditions. In some embodiments, each of the nanoparticles comprises 1 to 5 μg of the β₂ adrenergic agonist per mg of nanoparticles. In some embodiments, the composition further comprises a pharmaceutically acceptable carrier.

The present disclosure is not limited to particular β₂ adrenergic agonists. Examples include but are not limited to, albuterol, formoterol, bitolterol, fenoterol, isoproterenol, levalbuterol, metaproterenol, pirbuterol, procaterol, ritodrine, terbutaline, arformoterol, bambuterol, clenbuterol, salmeterol, abediterol, carmoterol, indacaterol, olodaterol, vilanterol, isoxsuprine, mabuterol, or zilpaterol.

The present disclosure is not limited to particular polymers. In some embodiments, the polymer is biocompatible and biodegradable. Examples include but are not limited to, methyl ether-block-poly(lactide-co-glycolide) (PLGA), poly(vinyl alcohol), chitosan, poly(ε-caprolactone) and/or poly(ethylene glycol) methyl ether-block-poly(lactide-co-glycolide) (PLGA-PEG). In some embodiments, the PEG is amine modified, methyl end capped, and/or carboxy terminated. In some embodiments, the PLGA has an average molecular weight of 10,000 to 20,000 (e.g., 55,000) and the PEG has an average molecular weight of 2000 to 10,000 (e.g., 5,000).

The nanoparticles are made by any suitable method. One exemplary method comprises a) mixing a first solution comprising said β₂ adrenergic agonist with a second solution comprising an emulsion of said polymer in a solvent; b) emulsifying the first solution in the second solution; and c) removing the solvent. Exemplary methods are described in Example 1.

In some embodiments of the present invention, the compositions are administered alone, while in some other embodiments, the compositions are preferably present in a pharmaceutical formulation comprising at least one active ingredient/agent, as defined above, together with a solid support or alternatively, together with one or more pharmaceutically acceptable carriers and optionally other therapeutic agents. Each carrier must be “acceptable” in the sense that it is compatible with the other ingredients of the formulation and not injurious to the subject.

The present disclosure further provides pharmaceutical compositions (e.g., comprising the compounds described above). The pharmaceutical compositions of the present disclosure may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients. Pharmaceutical compositions of the present disclosure include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present disclosure, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present disclosure may be formulated into any of many possible dosage forms such as, but not limited to, tablets, capsules, liquid syrups, soft gels, suppositories, and enemas. The compositions of the present disclosure may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

The compositions of the present disclosure may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present disclosure, such as dyes, flavoring agents, preservatives, antioxidants, opacifiers, thickening agents and stabilizers. However, such materials, when added, should not unduly interfere with the biological activities of the components of the compositions of the present disclosure. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC50s found to be effective in in vitro and in vivo animal models or based on the examples described herein. In general, dosage is from 0.01 μg to 100 g per kg of body weight, and may be given once or more daily. weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the oligonucleotide is administered in maintenance doses, ranging from 0.01 μg to 100 g per kg of body weight, once or more daily, to once every 20 years.

In certain embodiments, the present invention provides instructions for administering compositions described herein. In certain embodiments, the present invention provides instructions for using the compositions contained in a kit for the treatment or prevention of kidney disease (e.g., providing dosing, route of administration, decision trees for treating physicians for correlating patient-specific characteristics with therapeutic courses of action). In certain embodiments, the present invention provides instructions for using the compositions contained in the kit to treat a variety of medical conditions associated with kidney disease.

In some embodiments, in vivo administration is affected in one dose, continuously or intermittently throughout the course of treatment. Methods of determining the most effective means and dosage of administration are well known to those of skill in the art and vary with the composition used for therapy, the purpose of the therapy, the target cell being treated, and the subject being treated. Single or multiple administrations are carried out with the dose level and pattern being selected by the treating physician.

The present invention also includes methods involving co-administration of the agents described herein with one or more additional active agents. Indeed, it is a further aspect of this invention to provide methods for enhancing existing therapies and/or pharmaceutical compositions by co-administering agents described herein. In co-administration procedures, the agents may be administered concurrently or sequentially. In one embodiment, the agents described herein are administered prior to the other active agent(s). The pharmaceutical formulations and modes of administration may be any of those described above. In addition, the two or more co-administered chemical agents, biological agents or radiation may each be administered using different modes or different formulations. When the agents described herein are co-administered with another agent (e.g., as sensitizing agents), the effective amount may be less than when the agent is used alone.

In some embodiments, compositions described herein find use in treating or preventing kidney disease. As described herein, the formulations of the present disclosure allow the nanoparticles to localize to the kidney of the subject (e.g., to the tubules of the renal cortex). In some embodiments, the composition is administered to glomerular cells (e.g., podocytes, mesangial cells, endothelial cells) of the kidney. In addition, the administration does not result in cardiac side effects (e.g., tachycardic or hypotensive side effects).

The present disclosure is not limited to particular kidney diseases or damage. Examples include but are not limited to, acute renal injury, chronic renal injury, glomerular injury, drug and toxicant induced renal injury, ischemia-reperfusion injury, or diabetic nephropathy.

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

### EXPERIMENTAL

### Example 1

### Methods:

### Preparation of Drug PLGA-PEG Nanoparticles:

Formoterol containing Poly(ethylene glycol) methyl ether-block-poly(lactide-co-glycolide) (PLGA-PEG) (PLGA average Mn 55,000, PEG average Mn 5,000, Sigma-Aldrich, Saint Louis, MO) nanoparticles were prepared using the oil-in-water emulsion solvent evaporation method (Song C X, Labhasetwar V, Murphy H, Qu X, Humphry W R, Shebuski R J, et al. Formulation and characterization of biodegradable nanoparticles for intravascular local drug delivery Journal of Controlled Release 1997. p. 197-212). Briefly, 50 mg of PLGA-PEG was dissolved in a chloroform-acetonitrile solution (80:20). Formoterol fumarate dihydrate (Sigma-Aldrich, Saint Louis, MO) is dissolved in glacial acetic acid and 10 μL of formoterol solution is added to form the organic phase which is emulsified in an aqueous solution of 3% polyvinyl alcohol (PVA) using a microtip probe sonicator (Qsonica, Newtown, CT) at 60 W of energy output for 3 minutes over an ice bath. The emulsion was then diluted with 1% poloxamer 407 and organic phase removed by rotovapping at 40° C. for 40 minutes. Nanoparticle suspension is centrifuged at 15,000 rcf and washed thrice with distilled water. Samples are lyophilized (−80° C., <0.133 mmHg Labconco, Kansas City, MO) in 5% α-Trehalose and stored at −20° C. until use.

### Drug Loading and Encapsulation Efficiency:

An HPLC system (Alliance 2965, Waters, Milford, MA) with UV detector (Alliance 2487 Dual Wavelength Absorbance Detector, Waters, Milford, MA) with Phenomenex C18(2) column (4.6 mm×250 mm, 5 μm) was used to quantify formoterol encapsulated within the nanoparticles. The mobile phase consisted of methanol and 50 mM phosphoric acid buffer with 1% acetic acid at a ratio of 65:35, 1.0 mL/min flow rate and a column temperature of 40° C. The detection wavelength was 242 nm and the injection volume was 10 μL.

The drug loading (DL %) was calculated (Zhang Z, Feng S S. The drug encapsulation efficiency, in vitro drug release, cellular uptake and cytotoxicity of paclitaxel-loaded poly(lactide)-tocopheryl polyethylene glycol succinate nanoparticles. Biomaterials. 2006; 27(21):4025-33) as:

$$DL \% = \frac{the amount of formoterol assayed}{the total amount of nanoparticles in the preparation} \times 100$$

$$The encapsulation efficiency ( EE \% ) was calculated as :$$

$$EE \% = \frac{the amount of formoterol assayed}{the total amount of formoterol used in the preparation} \times 100$$

### LCMS Analysis:

Analysis of the formoterol content in the renal cortex was carried out using d-6 formoterol (Clearsynth, Mumbai, India) as an internal standard. Homogenization and extraction of formoterol from the renal cortex in methanol followed by centrifugation and HPLC separation was carried out as previously described (Mascher D G, Zech K, Nave R, Kubesch K M, Mascher H J. Ultra-sensitive determination of Formoterol in human serum by high performance liquid chromatography and electrospray tandem mass spectrometry. J Chromatogr B Analyt Technol Biomed Life Sci. 2006; 830(1):25-34). Quantification of formoterol was carried out using a Orbitrap Exploris 480 (ThermoFisher, Waltham, MA) mass spectrometer using the ESI source in the positive multiple reaction monitoring (MRM) mode. The MRM transitions were 345.3→149.1 m/z for formoterol and 351.3→155.1 m/z for d6-formoterol.

### In-Vitro Drug Release:

Formoterol loaded nanoparticles were dispersed in 10 mL of phosphate buffered saline (PBS) (pH 7.4) and incubated at 37° C. under magnetic stirring. At determined time intervals, aliquots were centrifuged at 10,000 rcf for 10 minutes. Supernatant was extracted and replaced with fresh PBS. Samples were analyzed by HPLC as described above. Nanoparticle release was determined in triplicate.

### Hydrodynamic Particle Size and Zeta Potential:

Formoterol containing PLGA-PEG nanoparticle suspensions were diluted to 1 mg/mL with deionized water. Particle size was determined by photon correlation spectroscopy using the Zetasizer Nano ZS (Malvern Instruments Ltd., Malvern, UK). The nanoparticle suspension was evaluated using a scattering angle of 173° at a temperature of 25° C. A minimum of 10 measurements were taken per replicate and samples were evaluated a minimum of 3 replicates.

Zeta potential measurements were carried out in 0.1× normal saline solution at 25° C. and a pH of 7.2. The mean zeta potential was determined using the Zetasizer Nano ZS (Malvern Instruments Ltd., Malvern, UK) phase analysis light scattering technique.

### Scanning Electron Microscopy:

The size and surface morphology of formoterol containing PLGA-PEG nanoparticles was captured using scanning electron microscopy (FEI Inspect S SEM, FEI Company, Hillsboro, OR) at 30 kV. Nanoparticle were deposited on conductive tape and sputter-coated with gold for 90 seconds before being loaded into the microscope.

### Renal Proximal Tubule Isolation and Culture:

Female New Zealand White rabbits were purchased from Charles River (Oakwood, MI). Renal proximal tubule cells (RPTCs) were isolated as previously described using the iron oxide perfusion method (Nowak G, Schnellmann R G. Improved culture conditions stimulate gluconeogenesis in primary cultures of renal proximal tubule cells. Am J Physiol. 1995; 268(4 Pt 1):C1053-610). The RPTCs were cultured on 35-mm tissue culture dishes with 1:1 DMEM:F-12 (without glucose, phenol red, or sodium pyruvate) as media supplemented with 15 mM HEPES buffer, 2.5 mM L-glutamine, 1 μM pyridoxine HCL, 15 mM sodium bicarbonate, and 6 mM lactate. Daily, hydrocortisone (50 nM), selenium (5 ng/mL), human transferrin (5 μg/mL), bovine insulin (10 nM), and L-ascorbic acid-2-phosphate (50 μM) were added to fresh culture medium. Confluent cells were used for all experiments.

### Cell Viability:

Cultured RPTCs were grown on 96-well plates at a cell density of 5×10³ cells/well for 96 h. Cells were exposed to varying concentrations of formoterol free drug as well as nanoparticle carrier. The treatments were each prepared by dissolving or suspending the powders in culture media. Following 24 hours of incubation at 37° C. and 5% CO2 with gentle shaking, the treatment was removed and cells were rinsed and replenished with fresh media. Viability was assessed (n=6 technical replicates per biological replicate) by resazurin reduction assay as described previously (41). Detection was assessed using Synergy H1 Multi-Mode Reader (BioTek Instruments, Inc., Winooski, VT) with 544 nm excitation and 590 nm emission wavelengths. The relative viability of the cells was calculated as follows:

$$Relative viability ( \% ) = \frac{Sample fluorescence intensity}{Control fluorescence intensity} \times 100 \%$$

### Animal Experiments:

Male C57BL/6 mice were obtained from Charles River (Oakwood, MI) and housed in a room at a constant temperature of 22+2° C. with 12:12 hour light-dark cycles. Mice 8-9 weeks of age were treated with saline vehicle, formoterol (0.3 mg/kg), or formoterol containing nanoparticles (0.04 mg/kg formoterol) via lateral tail vein injection. At 24 hours post exposure, mice were euthanized and kidney and heart were harvested. All experiments were approved by The University of Arizona in accordance with the guidelines set forth by the National Institutes of Health Guide for the Care and Use of Laboratory Animals.

### Immunoblot Analysis:

Protein was extracted from renal cortical tissue sections using radioimmunoprecipitation assay (RIPA) buffer (50 mM Tris-HCl, 150 mM NaCl, 0.1% SDS, 0.5% sodium deoxycholate and 1% Triton X-100, pH 7.4) with protease inhibitor cocktail (1:100), 1 mM sodium fluoride, and 1 mM sodium orthovanadate (Sigma Aldrich, St. Louis, MO) added prior to extraction. Equal protein quantities (10 μg) were loaded onto 4-15% SDS-PAGE gels, resolved by gel electrophoresis and transferred onto nitrocellulose membranes (Bio-Rad, CA). Membranes were blocked with 5% nonfat milk in Tris-buffered saline with Tween 20 to prevent nonspecific binding and incubated at 4° C. overnight with primary antibody. Primary antibodies include PGC-1α (EMD Millipore, Billerica, MA), mt-ND1, NDUFS1, and GAPDH (Abcam, Cambridge, MA). Membranes were washed and incubated with the appropriate horseradish peroxidase-conjugated secondary antibody (Santa Cruz biotechnology) for 1 h. Bound antibodies were visualized using enhanced chemiluminescence (Thermo Scientific, Waltham, MA) and the GE ImageQuant LAS400 (GE Life Sciences, Marlborough, MA).

### Immunofluorescence:

Kidney sections were harvested and fixed in 4% paraformaldehyde and embedded in paraffin. Embedded sections (5 μm thick) were deparaffinized using CitriSolv followed by rehydration in an ethanol gradient. Upon hydration, sections were permeabilized in 0.1% Triton X-100 for 10 minutes. Endogenous peroxidases were neutralized with 3% hydrogen peroxide. Slides were then blocked with normal horse serum in PBS for 1 h at room temperature. Polyethylene glycol (PEG) primary rabbit antibody (Abcam, Cambridge, MA) was added to slides at a 1:200 dilution and allowed to incubate overnight at 4° C. in a humidified environment. Slides were washed with PBS and incubated for 1 h with Alexa Fluor 594 secondary antibody (ThermoFisher, Waltham, MA) followed by Lectin phytohemagglutin-L conjugated to Alexa Fluor 488 (ThermoFisher, Waltham, MA) and DAPI nuclear dye (ThermoFisher, Waltham, MA). VECTASHIELD Antifade Mounting Medium was applied to each section along with a glass coverslip. All images were acquired using the EVOS M5000 Imaging System (ThermoFisher, Waltham, MA).

### Electron Microscopy:

Renal cortical tissue sections were fixed in glutaraldehyde, stained and sectioned for transmission electron microscopy as previously described (Isoe J, Collins J, Badgandi H, Day W A, Miesfeld R L. Defects in coatomer protein I (COPI) transport cause blood feeding-induced mortality in Yellow Fever mosquitoes. Proc Natl Acad Sci USA. 2011; 108(24): E211-7). Images were captured using the FEI Tecnai Spirit microscope (FEI, Hillsboro, OR) operated at 100 kV. Mitochondrial count and size were analyzed using the analyze particles plug-in in ImageJ FIJI.

### Tail Cuff Heart Rate and Blood Pressure:

The blood pressure and heart rates were measured with a non-invasive tail cuff device (CODA8, Kent Scientific, CT) which uses a volume-pressure method as previously described (Wang Y, Thatcher S E, Cassis L A. Measuring Blood Pressure Using a Noninvasive Tail Cuff Method in Mice. Methods Mol Biol. 2017; 1614:69-73). All measurements were performed on restrained, conscious animals located on a heating pad following the manufacturers protocol. Mice were measured in one set of 20 measurements following 5 acclimatization cycles. Cycles which did not pass the internal software control or were outside normal temperature range (34-37° C.) were excluded. At least 10 valid measurements were taken per animal.

### Statistical Analysis:

Comparison between the difference of means was performed by one-way analysis of variance (ANOVA) with the Tukey or Dunnett's test applied post hoc for comparisons (Prism 9.0) where p values of 0.05 or less were considered significant.

### Results

### Physicochemical Characterization of Formoterol Containing Nanoparticles:

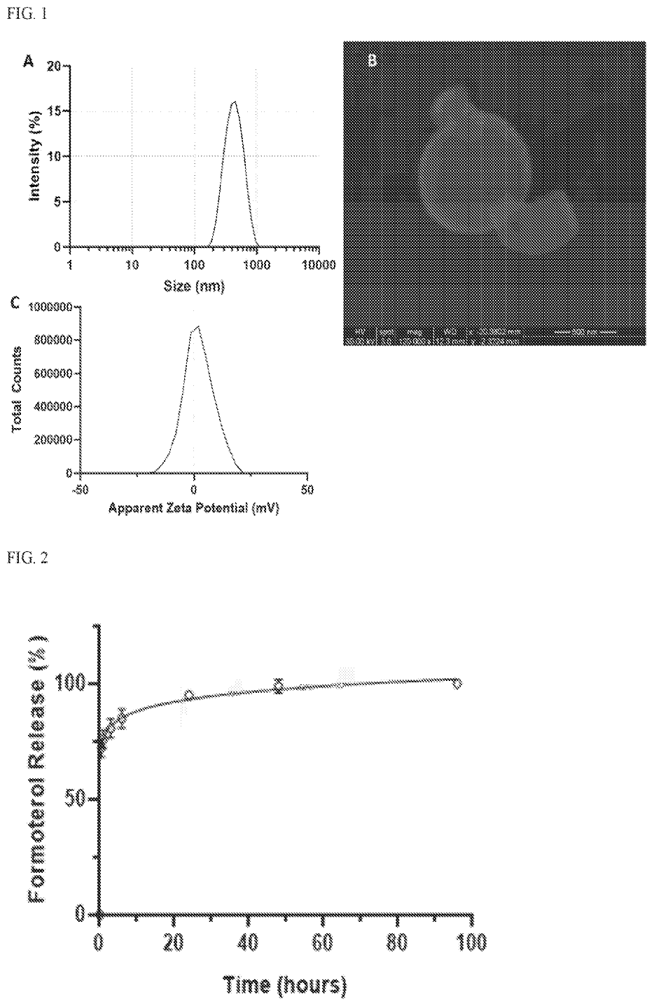

Nanoparticles prepared by single emulsion solvent evaporation procedures as described were resuspended following lyophilization and characterized for size, surface potential and drug loading (n=3 replicates). The mean hydrodynamic particle size (FIG. 1A) was measured in triplicate to be 442+17 nm with a polydispersity index of 0.1. The mean zeta potential (FIG. 1C) of the nanoparticles was measured to be 0.16+0.04 mV. Surface morphology of the nanoparticles (FIG. 1B) is shown to be smooth and the particles spherical in nature. These particles contained 1.6 μg per mg of nanoparticles or 0.2+0.04% drug loading with an entrapment efficiency of 24+5%. The in vitro release profile of formoterol containing nanoparticles was investigated in PBS at 37° C. The cumulative formoterol released is shown in FIG. 2. After the initial burst release within the first 3 hours, the release rate slows. The profile indicates >80% of the total drug release occurs within the first 24 hours with >99% of the drug release occurring within the first 48 hours.

### In Vitro RPTC Toxicity Study:

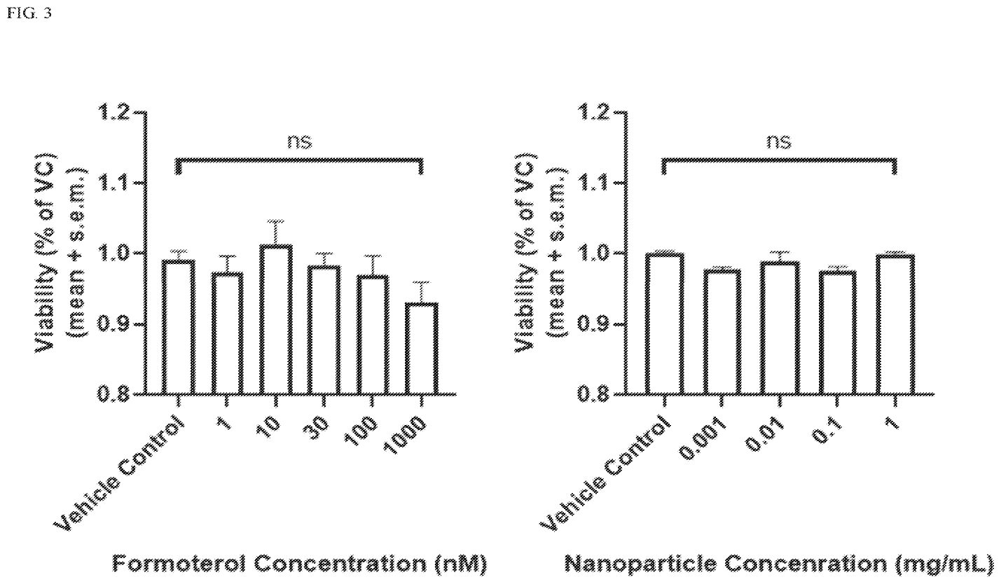

Confluent primary renal proximal tubule cells (n=3 biological replicates) treated with either formoterol or nanoparticles at concentrations up to 100× the intended exposure were assessed for any cytotoxic effects using the resazurin reduction assay. The viability of proximal tubule cells exposed to formoterol concentrations (1-1000 nm) (FIG. 3A) was not significantly decreased compared to the control following 24 hours of exposure. The viability of proximal tubule cells exposed to nanoparticle concentrations (0.001-1 mg/mL) (FIG. 3B) corresponds to the amount of nanoparticle needed for maximum formoterol concentration and shows no significant decrease following 24 hours of exposure.

### Renal Tubule Uptake of Formoterol Containing Nanoparticles:

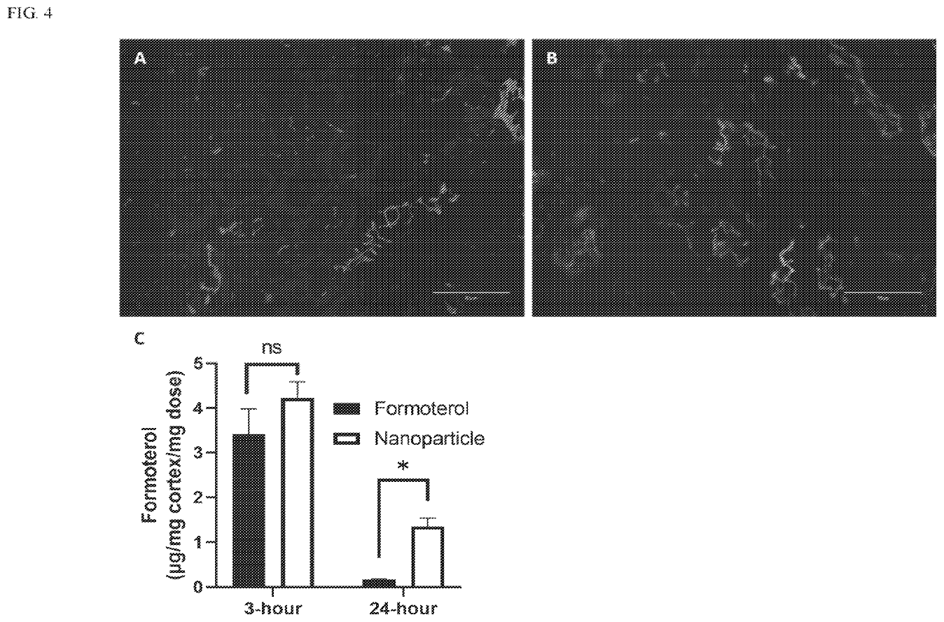

To demonstrate renal uptake of nanoparticles, 24 hours following administration of 60 mg/kg formoterol containing nanoparticles and formoterol to mice, renal cross-sections were prepared and stained using anti-PEG antibody. The proximal tubule localization was evaluated by co-staining with phytohemagglutinin lectin and DAPI. Results (FIG. 4A, representative n=3) show uptake of nanoparticles to the renal proximal tubule segments, as well as other tubule segments, however not endothelial cells or glomeruli. Additionally, these results show that the nanoparticles are present in the kidney up to 24 hours post-administration. Analysis of the concentration of formoterol in the renal cortex at 3- and 24-hours post-administration (n=4-5) was normalized to the amount of formoterol that was dosed. Data shows a significant difference in the amount of formoterol present in the kidneys between dosage forms (FIG. 4C). Free formoterol injections showed a substantial decrease in formoterol concentration by 24 hours with significantly lower concentration remaining in the renal cortex compared to formoterol containing nanoparticles.

### Renal Cortex and Ventricular Induction of Mitochondrial Biogenesis in Mice:

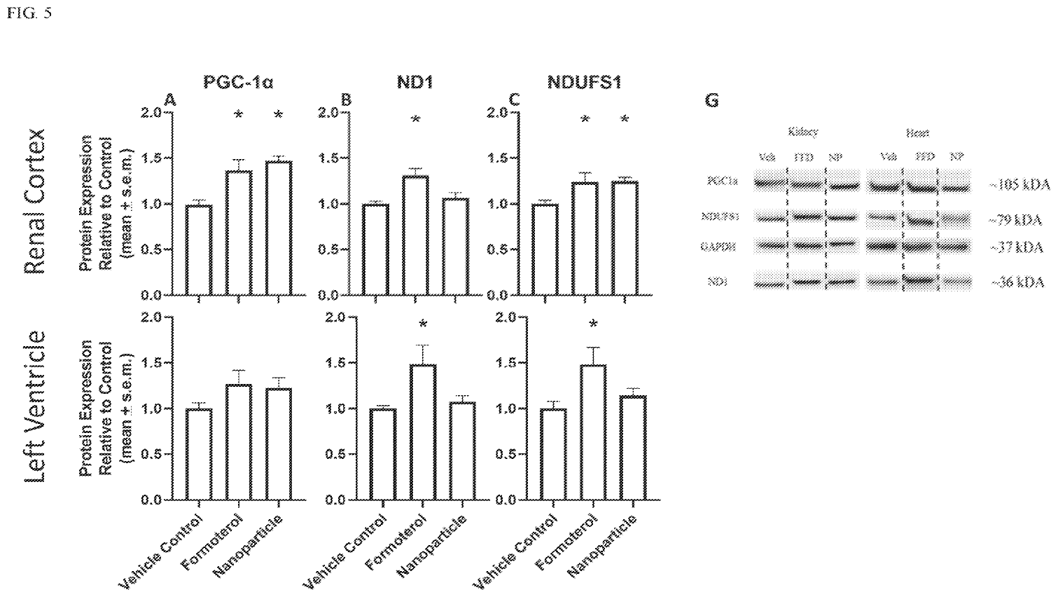

The renal cortex and left ventricle of mice (n=8-9) injected with formoterol free drug (0.3 mg/kg) or formoterol containing nanoparticles (0.04 mg/kg formoterol) were assessed following 24 hours of exposure for protein biomarkers of mitochondrial biogenesis. Overexpression in the renal cortex of the master regulator of mitochondrial biogenesis, PGC-1α, was exhibited for both formoterol free drug as well as formoterol containing nanoparticle treated animals (FIG. 5A). Both groups are significantly increased compared to the vehicle control. Expression of NADH dehydrogenase 1 (ND1) and NADH:Ubiquinone Oxidoreductase Core Subunit S1 (NDUFS1), both complex I proteins, are elevated in the renal cortex of mice treated with formoterol free drug and formoterol containing nanoparticles (FIGS. 5B & C). Both formoterol free drug and formoterol containing nanoparticles resulted in significantly increased expression of NDUFS1. Formoterol free drug resulted in significant increases in ND1 protein expression in the renal cortex while formoterol containing nanoparticles resulted in elevated but not significant ND1 expression. In the left ventricle, all three markers were elevated for formoterol free drug treated animals. For mice treated with formoterol, there was a significant change in complex I subunit expression (FIG. 5E-F). Formoterol free drug shows consistently higher cardiac expression of ETC proteins than the nanoparticle formulations. Elevated protein expression was significant for formoterol free drug when compared to control, but not for formoterol containing nanoparticles.

In order to confirm mitochondrial biogenesis directly, tissue sections of renal proximal tubules of mice (n=10) treated with vehicle, formoterol free drug or formoterol containing nanoparticles for 24 hours were imaged by TEM (FIG. 6A-C) and their mitochondria counted and mitochondrial area measured. Compared to the vehicle control, the total number of mitochondria counted (FIG. 6D) increased significantly with either formoterol free drug or formoterol containing nanoparticles as did the individual mitochondrial area (FIG. 6E).

### Formoterol Containing Nanoparticles Attenuates the Cardiovascular Effects of Formoterol:

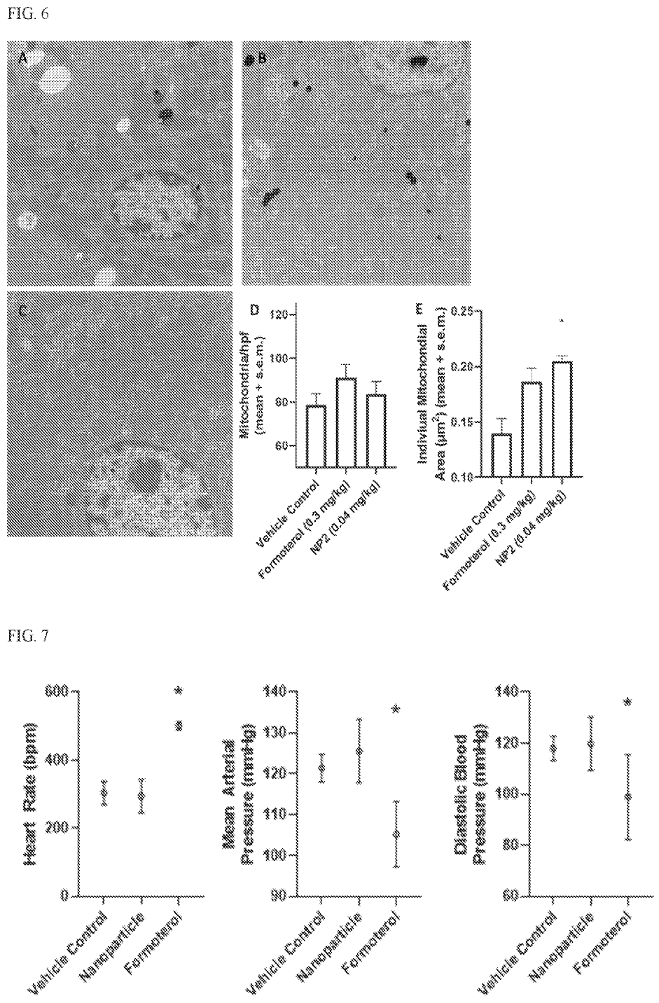

In order to assess the cardiovascular effects of formoterol free drug and formoterol containing nanoparticles on heart rate and blood pressure, mice were treated with either vehicle, formoterol (0.3 mg/kg) or formoterol containing nanoparticles (0.04 mg/kg formoterol) and heart rate and blood pressure was measured between 1-2 hours following administration. The heart rate of mice treated with formoterol free drug was significantly elevated, 198 bpm above the vehicle group or a 65% increase. In contrast, compared with the vehicle control, the formoterol containing nanoparticles showed a negligible change, a 9 bpm decrease or 3% (FIG. 7A). Unlike the changes in heart rate, formoterol free drug showed a decrease in mean arterial pressure (MAP) which was decreased by 16 mmHg and diastolic blood pressure which was decreased by 19 mmHg; both effects are significant. Formoterol containing nanoparticles showed no similar effects on either MAP or diastolic blood pressure which increased by 4 mmHg and 2 mmHg respectively (FIGS. 7B & C).

### Example 2

### Materials and Methods

### Materials

Formoterol fumarate dihydrate [C₁₉H₂₄N₂O₄·0.5C₄H₄O₄·H₂O; 420.46 g/mol; 99.8% pure] was purchased from APAC Pharmaceuticals (Columbia, MD, USA). Poly(ethylene glycol) methyl ether-block-poly(lactide-co-glycolide) (PLGA-PEG), lactide:glycolide ratio 50:50, PLGA average Mₙ 55,000 g/mol, 30,000 g/mol, and 15,000 g/mol; PEG average Mₙ 5,000 g/mol was purchased from Sigma-Aldrich (St. Louis, MO, USA). Poly(lactide-co-glycolide) methyl ether block-poly(ethylene glycol)-amine (PLGA-PEG-HN₂) PLGA average Mₙ 20,000 g/mol, PEG average Mₙ 5,000 g/mol and poly(lactide-co-glycolide) methyl ether block-poly(ethylene glycol)-carboxylic acid (PLGA-PEG-COOH) PLGA average Mₙ 20,000 g/mol, PEG average Mₙ 5,000 g/mol were purchased from Nanosoft Polymers (Winston-Salem, NC, USA). Acetic acid (HPLC grade) was purchased from ThermoFisher (Waltham, MA). Ethanol (99.9% purity, HPLC grade), hydrochloric acid 1 N (ACS grade), sodium hydroxide 1 N (ACS grade), poly(vinyl alcohol) (PVA) (molecular weight 89,000-98,000 g/mol, >99% hydrolyzed, reagent grade), Pluronic F127™ (molecular weight˜12,600 g/mol, reagent grade), sodium choate hydrate (>99% purity), sodium deoxycholate (>99% purity) potassium phosphate monobasic (>99% purity), potassium phosphate dibasic (>98% purity), formic acid (97.5%-98.5% purity), d-mannitol (ACS grade) and sucrose (99.5% purity) were purchased from Sigma-Aldrich (St. Louis, MO). (+)-Trehalose dihydrate (387.32 g/mol) was purchased from Acros Organics (Fair Lawn, NJ, USA). Choloroform (ACS grade), anhydrous acetonitrile (LCMS grade), and methanol (LCMS grade) were purchased from Spectrum Chemical Mfg. Corp. (Gardena, CA, USA). 0.45 μm polyvinylidene fluoride (PVDF) and 0.45 μm polytetrafluoroethylene (PTFE) membrane filters were purchased from MilliporeSigma (Burlington, MA, USA).

### Solubility of Formoterol Fumarate Dihydrate in Aqueous and Organic Media

Solubility of formoterol fumarate dihydrate (APAC Pharmaceuticals, Columbia, MD) was determined in various common aqueous and organic media to determine their suitability for use in preparation of nanoparticles. Excess of formoterol fumarate dihydrate was added to a known volume of solvent. For variable pH samples, solution pH was adjusted with either hydrochloric acid or sodium hydroxide solutions (1 M Sigma-Aldrich, St. Louis, MO). Vials were rotated gently for 24 hours at 25° C. as previously described (Aodah A, Pavlik A, Karlage K, Myrdal P B. Preformulation Studies on Piperlongumine. PLOS One. 2016; 11(3):e0151707). Test solutions were filtered using 0.45 μm PVDF or PTFE membrane filters (MilliporeSigma, Burlington, MA) for aqueous and organic solvents respectively. Quantitative analysis of formoterol content was determined by high performance liquid chromatography (HPLC) as previously described (Akapo S O, Asif M. Validation of a RP-HPLC method for the assay of formoterol and its related substances in formoterol fumarate dihydrate drug substance. J Pharm Biomed Anal. 2003; 33(5):935-45). Briefly, a C18-column (4.6 mm×250 mm length, 5 μm pore size) (Phenomenex Torrance, CA), mobile phase consisting of methanol (Spectrum Chemical MFG Corp., Gardena, CA) and 50 mM phosphoric acid (Sigma-Aldrich, St. Louis, MO) buffer with 1% acetic acid (ThermoFisher, Waltham, MA) at a ratio of 65:35, 1.0 mL/min flow rate and a column temperature of 40° C. was used to quantify formoterol content.

### Preparation of Polymeric Nanoparticles

Nanoparticles were prepared by either single or double emulsion methods. For nanoparticles prepared by oil-in-water single emulsion, polymer was dissolved in chloroform and formoterol fumarate dihydrate dissolved in methanol before being added to the polymer solution. This was then emulsified in an aqueous solution of 3% PVA (Sigma-Aldrich, St. Louis, MO) using a microtip probe sonicator (Qsonica, Newton, CT) at 60 Watt of energy output for 3 minutes over ice and the organic solvent allowed to evaporate with stirring (700 rpm) at room temperature for at least 8 hours. For nanoparticles prepared by water-in-oil-in-water double emulsion, formoterol fumarate dihydrate was dissolved in aqueous media before being added to a solution of polymer in chloroform (Spectrum Chemical Mfg. Corp. Gardena, CA) at a 1:2 aqueous:organic ratio. An initial emulsion was formed by sonicating at 60 Watt using a microtip probe sonicator for 30 seconds before being added to a 3% PVA solution and sonicated again. The organic solvent was then allowed to evaporate with stirring at room temperature. For all nanoparticle syntheses, particles were collected by centrifugation at 15,000 relative centrifugal force (rcf) and washed thrice with distilled ultrapure water (18.2 MΩ·cm) (Milli-Q Plus, MilliporeSigma, Burlington, MA). Samples were lyophilized at −80° C. under a vacuum <0.133 mmHg (FreeZone 4.5 L, Labconco, Kansas City, MO) with or without cryoprotectant and stored at −20° C. until use.

### Effect of Polymeric Nanoparticle Synthesis Parameters on Formoterol Drug Loading

To determine drug loading, nanoparticles were dissolved in an acetonitrile solution and analyzed by HPLC method reported above. Drug loading was calculated, as previously reported (Zhang Z, Feng S S. The drug encapsulation efficiency, in vitro drug release, cellular uptake and cytotoxicity of paclitaxel-loaded poly(lactide)-tocopheryl polyethylene glycol succinate nanoparticles. Biomaterials. 2006; 27(21):4025-33), using Equation 1:

$$DL ( \% ) = \frac{the amount of formoterol assayed}{the total amount of nanoparticles in the preparation} \times 100 \qquad Equation 1$$

### Effect of Polymeric Nanoparticle Synthesis Parameters on Particle Size

Nanoparticle size was determined immediately following washing. For evaluation of lyophilized particles, approximately 1 mg of nanoparticle was suspended in ultrapure water and centrifuged for 10 minutes at 15,000 rcf to remove the cryoprotectant. Nanoparticles were suspended at a concentration of ˜1 mg/mL with ultrapure water and hydrodynamic particle size was determined by photon correlation spectroscopy using the Zetasizer Nano ZS (Malvern Instruments Ltd., Malvern, UK) under previously reported conditions (Duan J, Mansour H M, Zhang Y, Deng X, Chen Y, Wang J, et al. Reversion of multidrug resistance by co-encapsulation of doxorubicin and curcumin in chitosan/poly(butyl cyanoacrylate) nanoparticles. Int J Pharm. 2012; 426(1-2):193-201). The suspended nanoparticles were evaluated using a scattering angle of 173° at a temperature of 25° C. in triplicate with a minimum of 10 measurements taken per replicate.

### Impact of Nanoparticle Synthesis Parameters on Zeta Potential

Nanoparticle zeta potential (ζ) measurements were carried out in 0.1× normal saline solution at 25° C. and pH 7.2. The mean ζ was determined using the Zetasizer Nano ZS (Malvern Instruments Ltd., Malvern, UK) phase analysis light scattering technique.

### Impact of Nanoparticle Synthesis Parameters on In Vitro Drug Release

Nanoparticles prepared as described above were dispersed in 10 mL of phosphate buffered saline (PBS) (pH 7.4) and incubated at 37° C. with gentle stirring as previously described (Cheng J, Teply B A, Sherifi I, Sung J, Luther G, Gu F X, et al. Formulation of functionalized PLGA-PEG nanoparticles for in vivo targeted drug delivery. Biomaterials. 2007; 28(5):869-76). At determined intervals, an aliquot was taken and centrifuged at 15,000 rcf for 10 minutes. The supernatant was extracted and replaced with equal volume of fresh PBS in order to maintain sink conditions. Formoterol content was chemically analyzed and quantified by HPLC as described above. Modeling of formoterol in vitro drug release was carried out for three kinetic models, namely, the zero-order, first-order, and Korsmeyer-Peppas models. Zero-order kinetics were fit to equation 2:

Qₜ−Q₀=k₀t Equation 2

Where Qₜ is the amount of drug released after time t, Q₀ is the initial amount of drug in solution and k₀ is the zero order rate constant. First-order kinetics were fit to equation 3:

ln Qₜ=ln Q₀−k₁t Equation 3

Where Qₜ is the amount of drug released after time t, Q₀ is the initial amount of drug in solution and k₁ is the first order rate constant. Finally, release kinetics were fit to the Korsmeyer-Peppas model, equation 4:

Qₜ=ktⁿ Equation 4

Where Qₜ is the amount of drug released after time t, k is the rate constant and n is the diffusion exponent for drug release. Nanoparticle release was determined in triplicated (n=3) for each preparation. Data were plotted using Prism 9.0 (GraphPad® Software, San Diego, CA, USA).

### Characterization of Nanoparticle Surface Morphology

Nanoparticle size and surface morphology was visualized using SEM (FEI Inspect S SEM, FEI Company, Hillsboro, OR). Powders were deposited on double-sided carbon conductive adhesive tabs (Ted-Pella, Inc., Redding, CA, USA) attached to aluminum SEM stubs (Ted-Pella, Inc., Redding, CA, USA) and sputter-coated (Anatech Hummer 6.2, Union City, CA, USA) with gold for 90 seconds under argon plasma as previously reported (Alabsi W, Al-Obeidi F A, Polt R, Mansour H M. Organic Solution Advanced Spray-Dried Microparticulate/Nanoparticulate Dry Powders of Lactomorphin for Respiratory Delivery: Physicochemical Characterization, In Vitro Aerosol Dispersion, and Cellular Studies. Pharmaceutics. 2020; 13(1)).

### X-Ray Powder Diffraction (XRPD)

The crystallinity of PLGA-PEG-NH₂, formoterol fumarate dihydrate, sucrose, trehalose and lyophilized nanoparticles with and without cryoprotectant were examined using XRPD. The diffraction patterns of samples were collected at room temperature scanning between 5.0° and 70.0° (2θ) at a rate of 2.00° per minute using a Philips PANalytical X'Pert PRO MPD (Malvern Panalytical, Malvern, UK) equipped with copper X-ray source (Kα radiation with λ=1.5406 Å). Samples were loaded on zero background single crystal silicon holders as previously reported (Duan et al., 2012, supra; Alabsi et al., 2020, supra).

### Thermal Analysis of Lyophilized Nanoparticles

Thermal analysis was performed by differential scanning calorimetry (DSC) and cross-polarized hot stage microscopy (HSM). DSC analysis was conducted as previously reported (Tajber L, Corrigan D O, Corrigan O I, Healy A M. Spray drying of budesonide, formoterol fumarate and their composites—I. Physicochemical characterisation. Int J Pharm. 2009; 367(1-2):79-85; Jarring K, Larsson T, Stensland B, Ymén I. Thermodynamic stability and crystal structures for polymorphs and solvates of formoterol fumarate. J Pharm Sci. 2006; 95(5):1144-61). Thermal analysis and phase transition measurements for raw formoterol fumarate dihydrate, raw PLGA-PEG-NH₂ (20,000 MW PLGA, 5,000 MW PEG), raw sucrose, raw trehalose, and lyophilized PLGA-PEG-NH₂ using with or without either sucrose or trehalose as a cryoprotectant were studied. Thermograms were acquired using the TA Q1000 differential scanning calorimeter with RSC090 cooling accessory (TA Instruments, New Castle, DE, USA). A mass of between 1 and 5 mg of sample was weighed into anodized aluminum hermetic pans (TA Instruments) with an empty pan used as a reference. DSC measurements were performed at a heating rate of 10° C./min from 0 to 350° C. Ultrapure nitrogen gas was used as the purging gas at a rate of 50 mL/min. Analysis of thermograms was conducted using TA Universal Analysis (TA Instruments). All measurements were carried out in triplicate.

Solid-state phase transitions of the lyophilized nanoparticles were observed using cross-polarized light HSM similarly to previously reported (Alabsi et al., 2020, supra). Microscopy was conducted using a Leica DMLP cross-polarized microscope (Leica Mircosystems, Wetzlar, Germany) equipped with a Mettler FP 80 central processor and FP82 hot stage (Mettler Toledo, Columbus, OH, USA). Lyophilized particles were mounted on a microscope slide and heated at a rate of 10° C./min from 25° C. to 300° C. The images were digitally captured using a Nikon Coolpix 8800 digital camera (Nikon, Tokyo, Japan) under 100× total magnification.

### Residual Water Content Analysis by Karl Fischer Titration

The residual water content of lyophilized nanoparticles was quantified by Karl Fischer titration (KFT) colorimetric assay using a TitroLine® 7500 trace titrator (SI Analytics, Mainz, Germany). Around 3-7 mg of sample was dissolved in 5 mL AQUA STAR anhydrous acetonitrile and injected into the titration cell. The measured moisture content was expressed in percentage as the result of the KFT. All measurements were completed in triplicate.

### Statistical Analysis

Comparison of the difference between three groups was performed by one-way analysis of variance (ANOVA) with Tukey's post hoc test for comparisons (Prism 9.0, GraphPad Software, San Diego, CA, USA). In all cases p values of 0.05 or less were considered significant.

### Results

### Solubility of Formoterol Fumarate Dihydrate in Aqueous and Organic Media

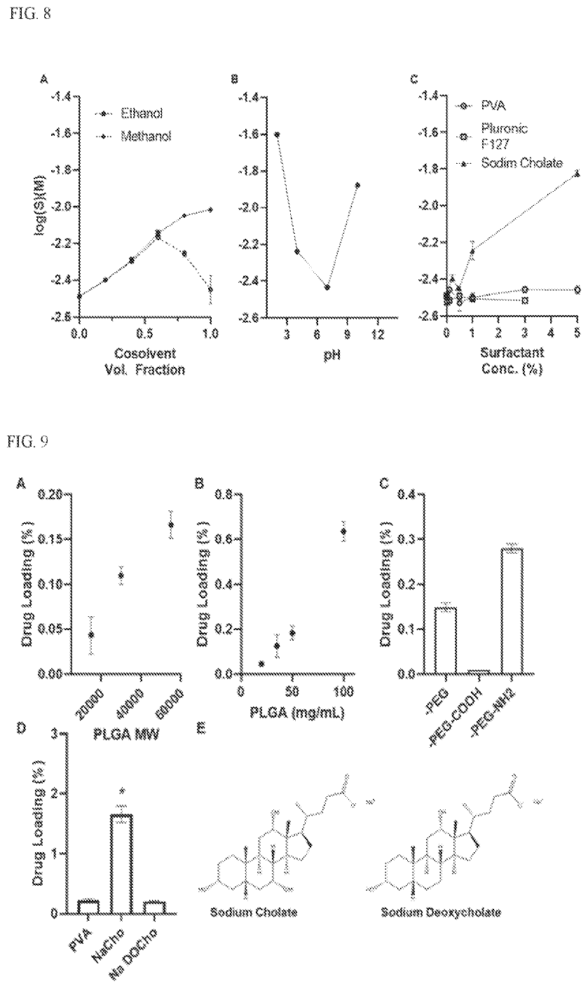

The solubility of formoterol and its salt formoterol fumarate dihydrate has previously been described in water and some organic media, however the solvents where published literature exists are those most commonly used in inhalation drug development such as various ionic and ethanol solutions, not the organic solvents most commonly used in the preparation of polymeric nanoparticles. The solubility of the fumarate salt of formoterol has a water solubility of 1.16±0.02 mg/mL at 25° C. Solubility is increased with increasing volume fraction of low molecular weight alcohols such as ethanol and methanol (FIG. 8A). Similar to previously reported studies, formoterol fumarate dihydrate likely forms a less soluble solvate with ethanol at volume fractions greater than 50% and sees a subsequent reduction in solubility with increasing cosolvent fraction. Similar solvent formation was not seen in water:methanol mixtures however this has been previously reported under different experimental conditions. Formoterol fumarate dihydrate sees increasing solubility in highly basic or acidic conditions (FIG. 8B) as formoterol fumarate dihydrate contains both acidic and basic pKa(s) around 8.6 and 9.8 respectively. Solubility of formoterol fumarate dihydrate in common non-ionic surfactants polyvinyl alcohol (PVA) and Pluronic® F127 solvent remains unchanged at concentrations ranging from 0.1% to 5% in aqueous solution. Solubilization is increased however in a concentration dependent manner above the critical micelle concentration of sodium cholate (12 mM or 0.52%), an ionic surfactant (FIG. 8C). Solubility in common organic solvents dichloromethane (DCM), chloroform, acetonitrile and acetate are also reported (Table 1), with acetone having the greatest solubility of 0.063±0.004 mg/mL.

### Effect of Polymeric Nanoparticle Synthesis Parameters on Formoterol Drug Loading

Achieving significant drug loading of formoterol in PLGA nanoparticles is complicated by the low solubility in organic media such as DCM and acetonitrile, where PLGA is freely soluble, compared to low molecular weight alcohols such as methanol, where PLGA and PLGA conjugates are practically insoluble. Drug loading of formoterol in PLGA-PEG nanoparticles prepared by single emulsion is improved with increasing PLGA molecular weight (35 mg/mL PLGA-PEG held constant), from 0.04% to 0.17% (FIG. 9A), and increasing PLGA-PEG concentration (using 55000 MW PLGA-PEG), up to 0.63% (FIG. 9B). This was the maximum drug loading achievable by a single emulsion solvent evaporation method. While the higher molecular weight polymer (55000 MW PLGA) achieved increased drug loading, the increases in drug loading are offset by increased nanoparticle size and reportedly increased degradation time in vivo (Chenthamara D, Subramaniam S, Ramakrishnan S G, Krishnaswamy S, Essa M M, Lin F H, et al. Therapeutic efficacy of nanoparticles and routes of administration. Biomater Res. 2019; 23:20; Mansour H M, Sohn M, Al-Ghananeem A, P.P. D. Materials for Pharmaceutical Dosage Forms: Molecular Pharmaceutics and Controlled Release Drug Delivery Aspects. International Journal of Molecular Sciences. 2010; 11(Special Issue-Material Sciences and Nanotechnology Section—Biodegradability of Materials.):3298-322; Rhee Y S, Park C W, DeLuca P P, Mansour H M. Sustained-Release Injectable Drug Delivery Systems. Pharmaceutical Technology: Special Issue-Drug Delivery. 2010 (November): 6-13) which may lead to accumulation and toxicity. For these reasons, 20000 MW PLGA polymers were selected for further study.

Formoterol loading is further enhanced by changing from single to double emulsion method and modification of the PEG terminus. Nanoparticles were prepared by water-in-oil-in-water double emulsion using PLGA-PEG, carboxylic acid modified PEG, or amine modified PEG. PLGA-PEG-COOH reduced drug loading compared to methyl terminated PEG from 0.15% to 0.01% whereas PLGA-PEG-NH₂ significantly increased drug loading to 1.39% (FIG. 9C).

Interaction between the formoterol and the inner aqueous surfactant was evaluated using 10 mg/mL 20000 MW PLGA-PEG-NH₂ and either 1% PVA, 12 mM sodium cholate or 10 mM sodium deoxycholate as the inner phase. The use of 10 mM sodium deoxycholate was required as stable nanoparticles did not form using 12 mM sodium deoxycholate. The use of the nonionic homopolymer surfactant PVA as the inner phase showed increased, 0.22%, drug loading compared to their equivalent single emulsion prepared particles (FIG. 9D). However, the use of the ionic surfactant sodium cholate showed a significant increase in drug loading, up to 1.66%. This increase in loading is completely nullified by the use of 10 mM sodium deoxycholate which differs from sodium cholate by only the 7α-hydroxyl group (structures FIG. 9E), indicating that this interaction is important to the improved loading seen by sodium cholate.

### Effect of Polymeric Nanoparticle Synthesis Parameters on Particle Size

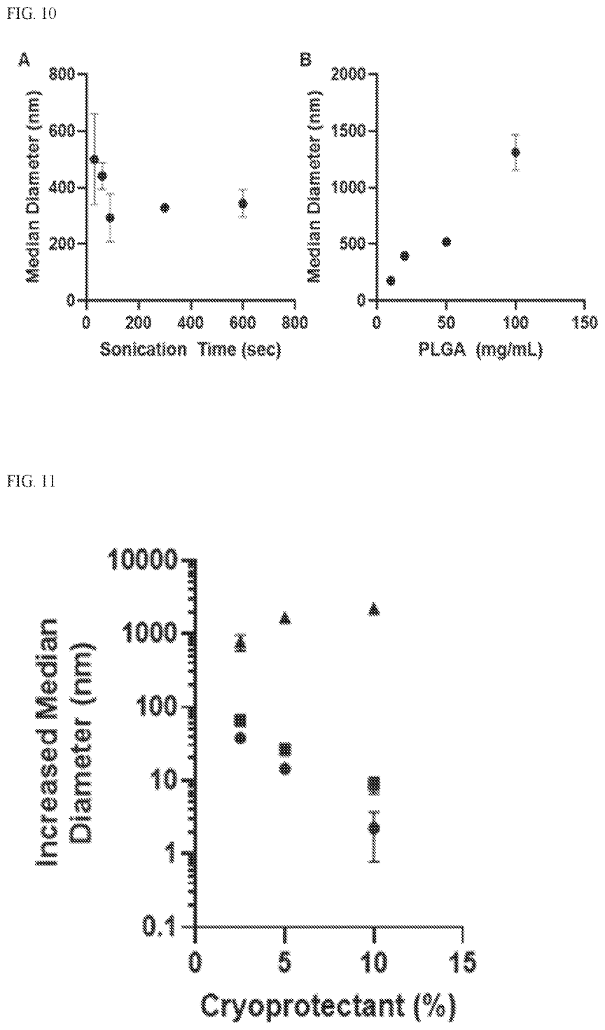

The impact of sonication and PGLA concentration on particle size was evaluated with the goal of achieving particles with median hydrodynamic diameters between 300 and 500 nm. Sonication time was the first parameter to be evaluated for double emulsion prepared particles (10 mg/mL PLGA-PEG-NH₂, 10 mM sodium cholate inner phase), increasing the secondary sonication from 30 to 600 seconds. Initially there is a precipitous decrease in median diameter, from over 500 nm to 292 nm with 90 seconds of sonication. Further increases in sonication time resulted in no significant change in particle size (FIG. 10A).

The impact of polymer concentration was additionally determined at concentrations of PLGA-PEG-NH₂ ranging from 10 to 100 mg/mL. All particles were prepared by double-emulsion, used 12 mM sodium cholate as an inner phase, and were sonicated for 180 seconds during preparation of the secondary emulsion. Particle size proved to be extremely sensitive to increasing PLGA concentration, with particle size increasing proportionally to the increase in polymer (FIG. 10B).

The impact of lyophilization was assessed following purification of the nanoparticles. To determine the impact of cryoprotectant selection and concentration on primary particle size, 1 mL of double-emulsion (12 mM sodium cholate inner phase, 180 second sonication) prepared PLGA-PEG-NH₂ nanoparticles at a concentration of 10 mg/mL were lyophilized in either 2.5, 5, or 10% of either sucrose, trehalose, or d-mannitol for 72 hours. Resuspended nanoparticles lyophilized with mannitol as the cryoprotectant showed significantly increased particle size, over 1 μm (FIG. 11). Conversely, both sucrose and trehalose cryoprotectants demonstrated decreased particle size growth post lyophilization with increasing cryoprotectant concentration, with sucrose performing slightly better than trehalose at all concentrations. For further characterization studies using nanoparticles lyophilized with cryoprotectants, the 5% cryoprotectant concentration was used.

### Impact of Nanoparticle Synthesis Parameters on Zeta Potential

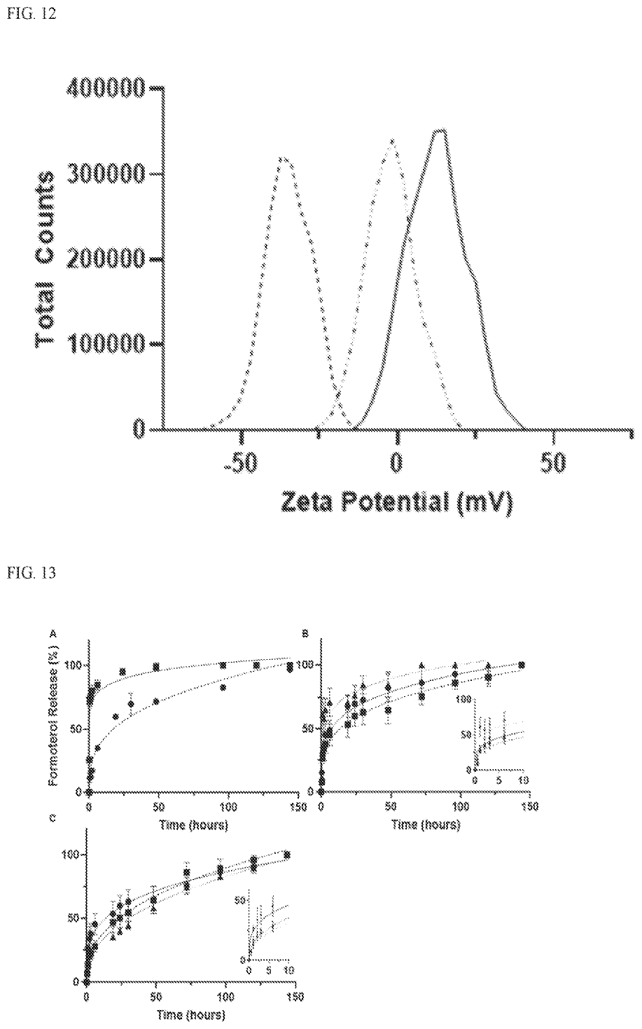

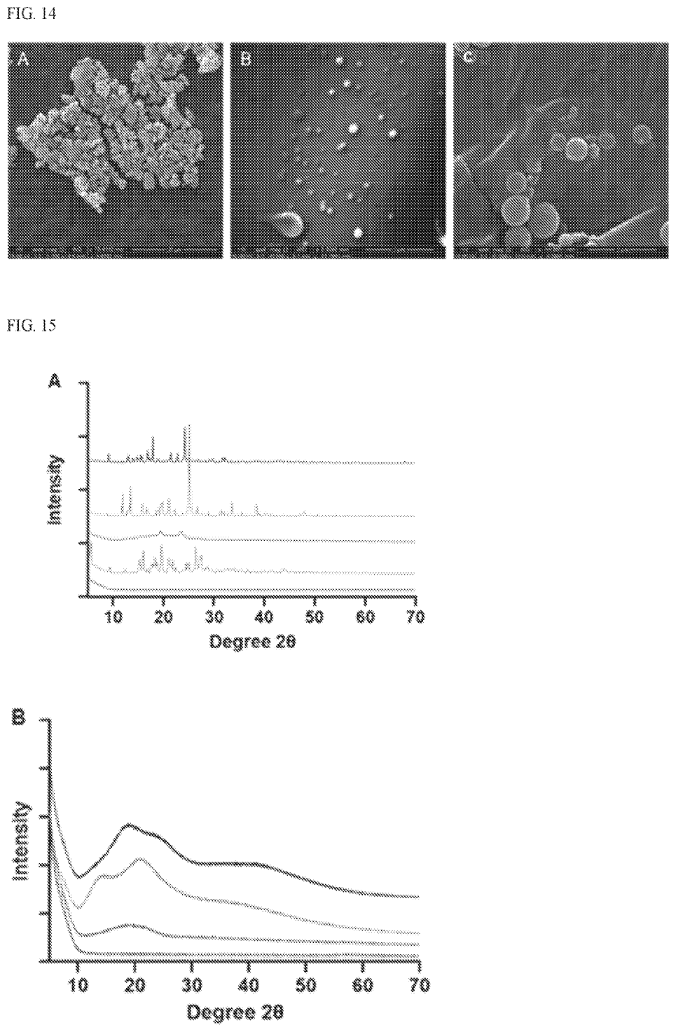

Nanoparticle zeta potential following washing was determined in 0.1× normal saline at 25° C. and neutral pH. Nanoparticle zeta potential was most strongly influenced by modification of PEG group (FIG. 12). Methyl endcapped PEG had a zeta potential of −0.792±0.284 mV, whereas amine modified PEG had a positive zeta potential of 14.133±0.404 mV and carboxylic acid modified PEG had a negative zeta potential of −34.87±0.945 mV.

### Impact of Nanoparticle Synthesis Parameters on Drug Release

Formoterol release is significantly altered by method of synthesis. Single and double emulsion solvent evaporation methods were assessed using 50 mg/mL 55000 MW PLGA-PEG, 180 seconds sonication time and using 1% PVA as the inner aqueous phase. Release was measured out to 1 week (144 hours) in PBS at 37° C. with constant stirring. Nanoparticles prepared by single emulsion showed significant burst release, with 80 and 90% of their entrapped drug released within the first 3 and 24 hours, respectively. Comparatively, nanoparticles prepared by double emulsion demonstrated a slower initial phase of release, with 3-hour release at 17% and 24-hour release between 60 and 70% (FIG. 13A). Analysis of the release kinetics showed non-fickian/anomalous diffusion (n>0.43) for single emulsion prepared nanoparticles and quasi-fickian diffusion for (n<0.43) for double emulsion prepared nanoparticles (Table 2).

Modification of the inner phase surfactant also impacted the initial release. Nanoparticle were prepared using 10 mg/mL 20000 MW PLGA-PEG-NH₂ and varying the inner phase between 1% PVA, 12 mM sodium cholate or 10 mM sodium deoxycholate. The PVA and sodium cholate prepared particles showed slight differences in 3-hour burse release (45 and 38% respectively) and both were significantly lower than the sodium deoxycholate prepared particles which showed 65% drug release by 3-hours (FIG. 13B). Analysis of release kinetics showed no significant difference in release exponent between PVA and sodium cholate inner phases, however there was a significant (p<0.05) difference between those and sodium deoxycholate (Table 2). These differences were less apparent during the sustained release phase (time>24 hours). Finally, increasing concentration of PLGA-PEG-NH₂ during nanoparticle synthesis using 12 mM sodium cholate as the inner phase resulted in a decreased percentage of formoterol released within the first 3 hours and increased rate of release beyond 24 hours (FIG. 13C). Increasing polymer concentration resulted in increasing the release exponent towards fickian (n=0.43) release (Table 2).

### Characterization of Nanoparticle Surface Morphology

The surface characteristics of nanoparticles prepared by double emulsion were assessed. Nanoparticles of PLGA-PEG with sodium cholate inner phase showed a high degree of surface roughness and size irregularity as well as a tendency to agglomerate (FIG. 14A). Nanoparticles prepared by double-emulsion with PLGA-PEG-COOH and PLGA-PEG-NH₂ with sodium cholate inner phase were similarly sized, producing spherical particles that did not form aggregates and had smooth surface features (FIGS. 14B & C).

### X-Ray Powder Diffraction (XRPD)

X-ray diffractograms of nanoparticle raw materials (PLGA-PEG-NH₂, formoterol fumarate dihydrate and cryoprotectants) as well as lyophilized nanoparticles with and without cryoprotection were obtained (FIG. 15). The diffraction pattern of raw materials formoterol fumarate dihydrate, sucrose and trehalose showed multiple sharp peaks across the scanned range indicating long range molecular order consistent with crystallinity (FIG. 8A). Raw polymer samples and all lyophilized prepared nanoparticles did not contain any sharp crystalline peaks (FIGS. 15A & B).

### Thermal Analysis of Lyophilized Nanoparticles

Thermal analysis of nanoparticle components formoterol fumarate dihydrate, PLGA-PEG-NH₂, sucrose and trehalose as well as lyophilized nanoparticles with or without cryoprotectants are summarized in (Table 3).

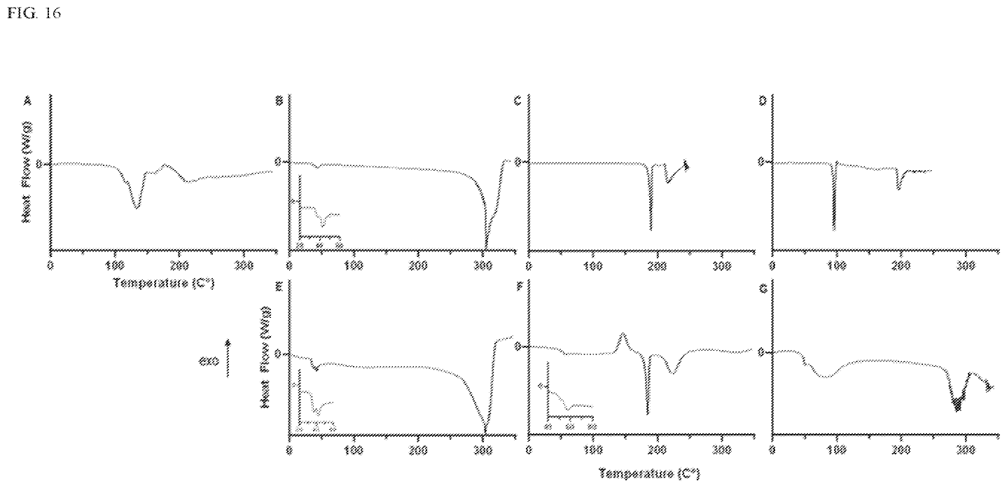

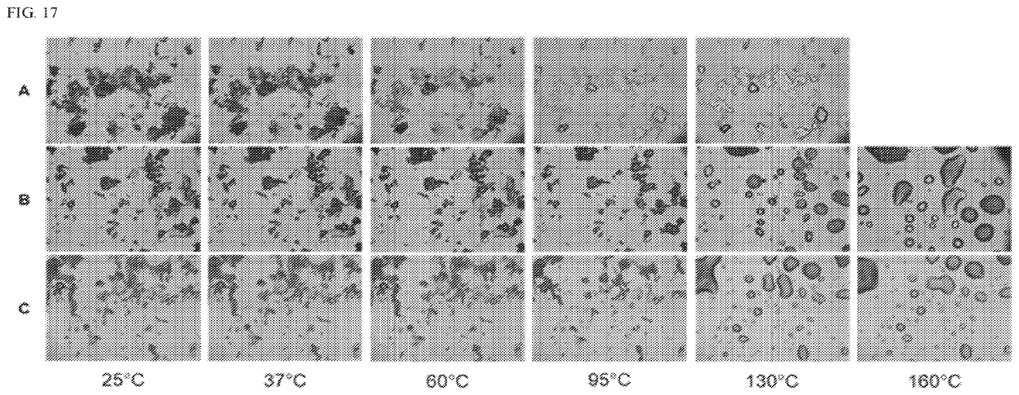

For formoterol fumarate dihydrate, there is a bimodal endotherm with an initial peak of 104° C. and main peak of 130° C. Above 150° C., thermal decomposition was seen in the form of a jagged baseline (FIG. 16A). For PLGA-PEG-NH₂ polymer, there is a clear glass transition peak (T_{g}) from 1-43° C. combined with an endotherm at 48° C. and a broad decomposition starting at 250° C. (FIG. 16B). Raw sucrose shows a sharp endotherm at 189° C. followed by a broad decomposition endotherm at 220° C. (FIG. 9C). Trehalose dihydrate showed a sharp endotherm at 95° C. followed by a broad endotherm at 193° C. followed by decomposition (FIG. 16D). PLGA-PEG-NH₂ particles lyophilized without cryoprotectant showed slightly decreased T_{g} of 35-36° C. compared to the raw polymer and a similar first endotherm at 44° C. followed by a broad endotherm starting from 82° C. and peaking at 112° C. (FIG. 16E). Nanoparticles lyophilized with 5% sucrose as a cryoprotectant showed an increased T_{g} and first endotherm comparted to the raw polymer, 50-54° C. and 57° C. respectively. Additionally, the broad endotherm at 96° C. transitioned into an exothermic peak at 148° C. followed by an endotherm at 184° C. and secondary endotherm at 222° C. leading to decomposition (FIG. 16F). Finally, nanoparticles lyophilized with trehalose as a cryoprotectant had a similar T_{g} to the raw polymer, 43-46° C. followed by a broad endotherm starting from 58° C. and peaking at 86° C. No other endotherms were detected until decomposition starting above 250° C.

Lyophilized nanoparticles were also evaluated by cross-polarized HSM. All three particles (lyophilized without cryoprotectant, with sucrose cryoprotectant and with trehalose cryoprotectant) were dark and lacked birefringence at 25° C. and 37° C. At 60° C., the cryoprotectant free particles begin melting, a process that will take until melting is fully completed at 130° C. (FIG. 17A). Both formulations lyophilized with cryoprotectants did not have observable melts until closer to 100° C. (FIGS. 17B & C). Nanoparticles lyophilized with sucrose exhibited a liquid crystal transition, as noted by the marked diffuse birefringence at 130° C. (FIG. 17B). Both lyophilates had completely melted by 160° C. and there were no observable transitions after that temperature.

### Water Content Analysis by Karl Fischer Titration

Water content was determined for double emulsion prepared lyophilates with and without cryoprotection (Table 4). For lyophilates of PLGA-PEG without cryoprotection, water content was 1.38±0.20%. Comparatively, amine modification of the PEG group increased water content to 2.20±0.61%. Lyophilization with 5% of either sucrose or trehalose resulted in significantly (p<0.05) reduced water content. 0.78±0.17% and 0.80±0.19% respectively.

TABLE 1
Solubility of formoterol in various organic solvents.

| Solvent | Formoterol Solubility (mg/mL) |
|---|---|
| DCM | 0.001 ± 0.0004 |
| Chloroform | 0.002 ± 0.001 |
| ACN | 0.005 ± 0.001 |
| Acetone | 0.051 ± 0.004 |

DCM; dichloromethane, ACN; acetonitrile.
Data are presented as the mean (n = 3) ± s.d.

TABLE 2
Nanoparticle release kinetics modeling. o/w; oil-in-water single
emulsion w/o/w; water-in-oil-in-water double emulsion, PVA;
nanoparticles prepared by double emulsion using 1% PVA inner
phase, NaCholate; nanoparticles prepared by double emulsion
using 12 mM sodium cholate inner phase, NaDOCholate; nanoparticles
prepared by double emulsion using 10 mM sodium deoxycholate
inner phase, 10/20/50 mg/mL; nanoparticles prepared by double
emulsion using 10/20/50 mg/mL of polymer in the organic phase.

| | | |
|---|---|---|
| Zero | First | |
| Order | Order | Korsmeyer-Peppas |

| | R² | R² | R² | n |
|---|---|---|---|---|
| o/w | 0.33 | 0.94 | 0.88 | 0.56 |
| w/o/w | 0.77 | 0.97 | 0.97 | 0.34 |
| PVA | 0.65 | 0.89 | 0.94 | 0.23 |
| NaCholate | 0.75 | 0.86 | 0.94 | 0.27 |
| NaDOCholate | 0.49 | 0.81 | 0.84 | 0.17 |
| 10 mg/mL | 0.75 | 0.86 | 0.94 | 0.27 |
| 20 mg/mL | 0.85 | 0.96 | 0.97 | 0.40 |
| 50 mg/mL | 0.92 | 0.96 | 0.98 | 0.42 |

All data are presented as the mean of (n = 3) release profiles.

TABLE 3
Differential scanning calorimetry (DSC) thermal analysis. T_{g}; glass transition temperature. Data presented are mean (n = 3) ± s.d.

| | | | | | | Raw | | |
|---|---|---|---|---|---|---|---|---|
| Nanoparticle a | Nanoparticle | Nanoparticle | | | Raw | Formoterol | | |
| Trehalose | Sucrose | No | Raw | Raw | PLGA- | Fumarate | | |
| Cryoprotectant | Cryoprotectant | Cryoprotectant | Trehalose | Sucrose | PEG-NH2 | Dihydrate | Sample | |
| 43.08 ± 3.39 | 50.09 ± 1.1 | 35.3 ± 1.25 | | | 41.56 ± 1.34 | | onset (° C.) | Tg (° C.) |
| 44.68 ± 4.02 | 54.3 ± 0.21 | 36.12 ± 0.8 | | | 42.43 ± 1.51 | | mid (° C.) | |
| 46.64 ± 2.94 | 54.82 ± 0.43 | 36.48 ± 0.69 | | | 43.34 ± 1.93 | | end (° C.) | |
| 1.21 ± 0.31 | 1.34 ± 0.38 | 0.3 ± 0.2 | | | 0.69 ± 0.44 | | J/g ° C. | Δ Cp |
| 42.97 ± 1.61 | 53.45 ± 0.32 | 42.16 ± 6.75 | | | 43.32 ± 2.16 | 88.84 ± 1.87 | onset (° C.) | Endotherm #1 |
| 50.17 ± 0.18 | 57.15 ± 0.33 | 43.99 ± 6.73 | | | 47.97 ± 4.72 | 104.62 ± 3.62 | peak (° C.) | |
| 4.73 ± 3.73 | 4.13 ± 0.43 | 1.98 ± 1.14 | | | 4.95 ± 3.34 | 13.56 ± 1.04 | enthalpy (J/g) | |
| 58.99 ± 1.99 | 70.93 ± 2.3 | 82.62 ± 2.34 | 94.46 ± 0.07 | | | 116.28 ± 0.76 | onset (° C.) | Endotherm #2 |
| 86.75 ± 3.45 | 96.02 ± 1.58 | 112.95 ± 9.6 | 95.79 ± 0.05 | | | 130.82 ± 2.73 | peak (° C.) | |
| 16.89 ± 2.72 | 10.11 ± 0.56 | 8.67 ± 3.24 | 94.54 ± 2.28 | | | 136.7 ± 0.89 | enthalpy (J/g) | |
| | 180.03 ± 0.36 | | 190.98 ± 1.75 | 187.01 ± 0.59 | | | onset (° C.) | Endotherm #3 |
| | 184.74 ± 0.03 | | 193.3 ± 3.24 | 189.43 ± 0.23 | | | peak (° C.) | |
| | 86.59 ± 2.38 | | 92.98 ± 14.66 | 129.47 ± 0.4 | | | enthalpy (J/g) | |
| | 213.08 ± 5.68 | | | 219.35 ± 5.56 | | | onset (° C.) | Endotherm #4 |
| | 222.78 ± 2.12 | | | 220.72 ± 4.48 | | | peak (° C.) | |
| | 119.77 ± 1.16 | | | 150 ± 50.01 | | | enthalpy (J/g) | |
| | 136.56 ± 1.93 | | | | | | onset (° C.) | Exotherm #1 |
| | 148.26 ± 3.06 | | | | | | peak (° C.) | |
| | 79.36 ± 5.03 | | | | | | enthalpy (J/g) | |

TABLE 4
Residual water content of lyophilized nanoparticles
with and without cryoprotectants.

| Nanoparticle | Water % (w/w) |
|---|---|
| PLGA-PEG (no cryoprotectant) | 1.38 ± 0.20 |
| PLGA-PEG-NH₂ (no | 2.20 ± 0.61 |
| cryoprotectant) | |
| PLGA-PEG-NH₂ (sucrose) | 0.78 ± 0.17* |
| PLGA-PEG-NH₂ (trehalose) | 0.80 ± 0.19* |

*indicates significantly different from other groups (p < 0.05) Data are presented as the mean (n = 3)

### Example 3

### Methods

### Synthesis and Characterization of Formoterol and Blank PLGA-PEG Nanoparticles

Polymeric nanoparticles containing formoterol (FNP) and blank vehicle nanoparticles (VNP) were prepared as previously described (Vallorz E L, Blohm-Mangone K, Schnellmann R G, et al. Formoterol PLGA-PEG Nanoparticles Induce Mitochondrial Biogenesis in Renal Proximal Tubules. AAPS J 2021; 23: 88). DEDC containing nanoparticles (DEDC-NP) were prepared similarly, replacing formoterol fumarate dihydrate with 1 mg/mL DEDC. The hydrodynamic size of the particles was determined via dynamic light scattering at 1 mg/mL in normal saline. Formoterol loading was ascertained for FNPs as described by HPLC-UV detection, according to published methods) Mascher D G, Zech K, Nave R, et al. Ultra-sensitive determination of Formoterol in human serum by high performance liquid chromatography and electrospray tandem mass spectrometry. J Chromatogr B Analyt Technol Biomed Life Sci 2006; 830: 25-34).

### Animal Use

Male C57Bl/6 mice (8-10 weeks of age) were obtained from Charles River (Oakwood, MI), housed in the University of Arizona Animal Care (UAC) Facility at a constant temperature of 22±2° C. with 12:12 hour light-dark cycles and received standard rodent food and water ad libitum.

### Ischemia-Reperfusion Induced Acute Kidney Injury

Mice were subjected to bilateral renal ischemia for 18-20 minutes or sham surgery as previously described (Funk J A, Schnellmann R G. Persistent disruption of mitochondrial homeostasis after acute kidney injury. Am J Physiol Renal Physiol 2012; 302: F853-864). Mice were allowed to recover without intervention for 24 hours and sorted into groups based on 24-hour serum creatinine levels prior to initiation of dosing. Mice were dosed either daily or at a reduced frequency at 24 hours and 96 hours post-injury (2-day dosing). Mice were dosed with either normal saline (VS), 30 mg/kg vehicle nanoparticles (VNP), 0.3 mg/kg formoterol fumarate dihydrate in normal saline (FFD), or 30 mg/kg nanoparticle containing 0.04 mg/kg formoterol (FNP). Mice were given VS and FFD via intraperitoneal injection. Mice were administered VNP and FNP via lateral tail intravenous injection.

### Renal Localization of Nanoparticles

Mice (n=5) were subjected to bilateral renal ischemia for 18-20 minutes as previously described (Funk et al., 2012, supra). Following 24 hours of reperfusion, mice were administered 30 mg/kg DEDC-NP by lateral tail vein injection. At 1 hour post administration, the kidneys, heart, lungs, liver, spleen and stomach of the mice were removed, weighted and imaged for fluorescence using the Lago live animal fluorescent imaging system (Spectral Instruments Imaging, Tucson, AZ) with 675 nm excitation and 710 nm emission filters. Analysis of the acquired images was carried out using Aura imaging software (Spectral Instruments Imaging, Tucson, AZ) to obtain the total fluorescence emission (TFE) which was then normalized to organ weight.

### Serum Creatinine and Biomarker Analysis

Blood was collected from mice at 24-, 96- and 144-hours post-injury by retro-orbital bleeding. Serum creatinine was quantified using the Creatinine Enzymatic Reagent Assay kit (Diazyme Laboratories, Inc. La Jolla, CA), according to the manufacturer's protocol. Serum KIM-1 and NGAL was quantified using the mouse KIM-1 and NGAL ELISA kits (Abcam, Cambridge, UK) according to the manufacturer's instructions.

### Protein Isolation and Immunoblotting

Protein was extracted from renal cortical tissue and the left ventricle of the heart using radioimmunoprecipitation assay (RIPA) buffer (50 mM Tris-HCl, 150 mM NaCl, 0.1% SDS, 0.5% sodium deoxycholate and 1% Triton X-100, pH 7.4) with protease inhibitor cocktail (1:100), 1 mM sodium fluoride, and 1 mM sodium orthovanadate (Sigma Aldrich, St. Louis, MO). Equal protein quantities (10 μg) were loaded onto 4-15% SDS-PAGE gels, resolved by gel electrophoresis, and then transferred onto nitrocellulose membranes. Membranes were blocked in 5% milk in tris-buffered saline/tween 20, incubated at a temperature of 4° C. for 18-24 hours with primary and secondary antibodies. Bound antibodies were visualized using enhanced chemiluminescence (Thermo Scientific, Waltham, MA) and the GE ImageQuant LAS400 Biomolecular Imaging System (GE Healthcare, Marlborough, MA).

### Electron Microscopy

At 144 hours post-injury, sections from the renal cortex of the mouse right superior renal pole were fixed in glutaraldehyde, stained, and sectioned for imaging by transmission electron microscopy (TEM) as previously described (Dupre T V, Jenkins D P, Muise-Helmericks R C, et al. The 5-hydroxytryptamine receptor 1F stimulates mitochondrial biogenesis and angiogenesis in endothelial cells. Biochem Pharmacol 2019; 169: 113644). Micrographs were obtained using the FEI Tecnai Spirt microscope (FEI, Hillsboro, OR) operated at an accelerating voltage of 100 kV. Mitochondrial count and size were analyzed using MATLAB 9.9 (R2020b) (Mathworks, Natick, MA). Acquisition and analysis of images was carried out by a researcher blinded to injury and treatment group.

### Histochemistry

Right kidneys of mice 144 hours post injury were fixed in 10% neutral buffered formalin solution and embedded in paraffin. Immunohistochemistry was performed by staining with KIM-1 primary antibody and incubated at a temperature of 4° C. for 18-24 hours followed by Alexa Fluor 488 (Thermo Scientific, Waltham, MA). Slides for histopathology were stained with periodic acid-Schiff (PAS) solutions (Thermo Scientific, Waltham, MA) and trichrome stain kit (Abcam). The degree of morphologic change was assessed by brightfield microscopy by a trained pathologist who was blinded to injury and treatment group. Tubular necrosis and severity of protein cast formation was evaluated on a scale from 0 to 4 which ranged from not present (0), mild (1), moderate (2), severe (3), and very severe (4). Tubulointerstitial fibrosis was determined by measuring the nuclei-free, collagen-positive area as a fraction of the renal cortex using ImageJ.

### Cardiovascular Function

Mouse blood pressure and heart rates were measured with a non-invasive tail cuff device (CODA8, Kent Scientific, CT) as previously described (Wang Y, Thatcher S E, Cassis L A. Measuring Blood Pressure Using a Noninvasive Tail Cuff Method in Mice. Methods Mol Biol 2017; 1614: 69-73). All measurements were performed on restrained, conscious animals located on a heating pad following the manufacturer's protocol. Mice were measured in one set of 20 measurements following 5 acclimatization cycles. Cycles which did not pass the internal software control or were outside normal temperature range (34-37° C.) were excluded. At least 10 valid measurements were taken per animal.

### Statistical Analysis

Data are expressed as mean±S.E.M. for all experiments. Statistical significance was determined via one- or two-way ANOVA and differences between groups was determined using Tukey post hoc test. The criteria for statistical significance were P<0.05 for all experiments.

### Results

### Synthesis and Characterization of Polymeric Nanoparticles Containing Formoterol

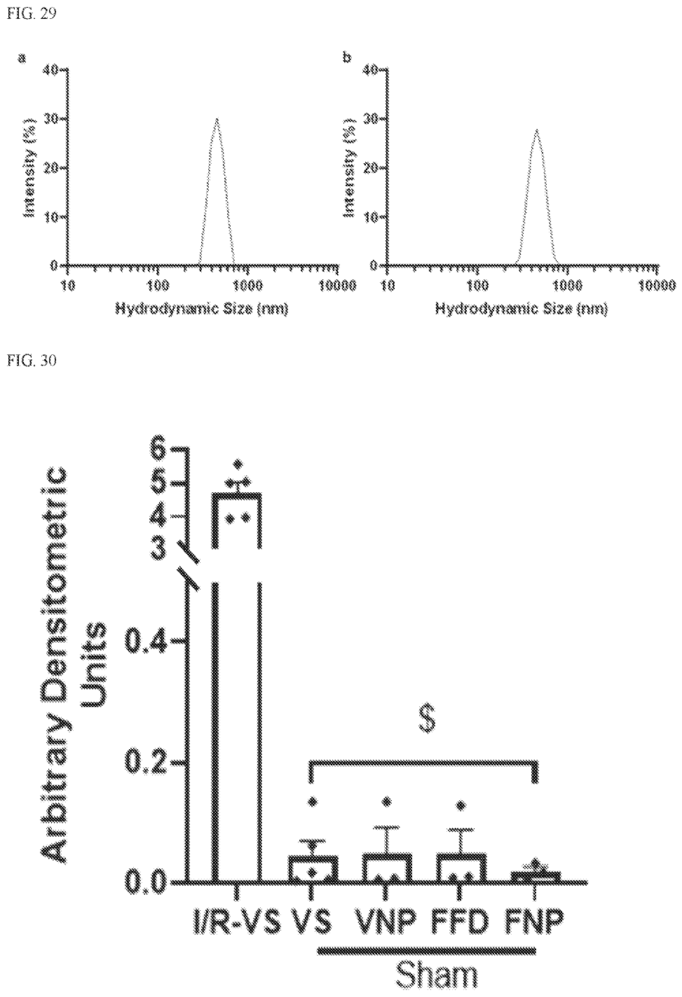

FNP and blank nanoparticles (VNP) were synthesized and lyophilized in 5% α-trehalose. Reconstituted FNP and VNP had a median hydrodynamic diameter of 463±40 nm and 493±24 nm, respectively (FIG. 29). The FNP encapsulated 1.33 μg formoterol per mg of nanoparticles. This drug loading allows for mice to be dosed at 30 mg/kg nanoparticle containing 0.04 mg/kg of formoterol.

### Nanoparticles Localize to the Kidneys Following I/R Injury

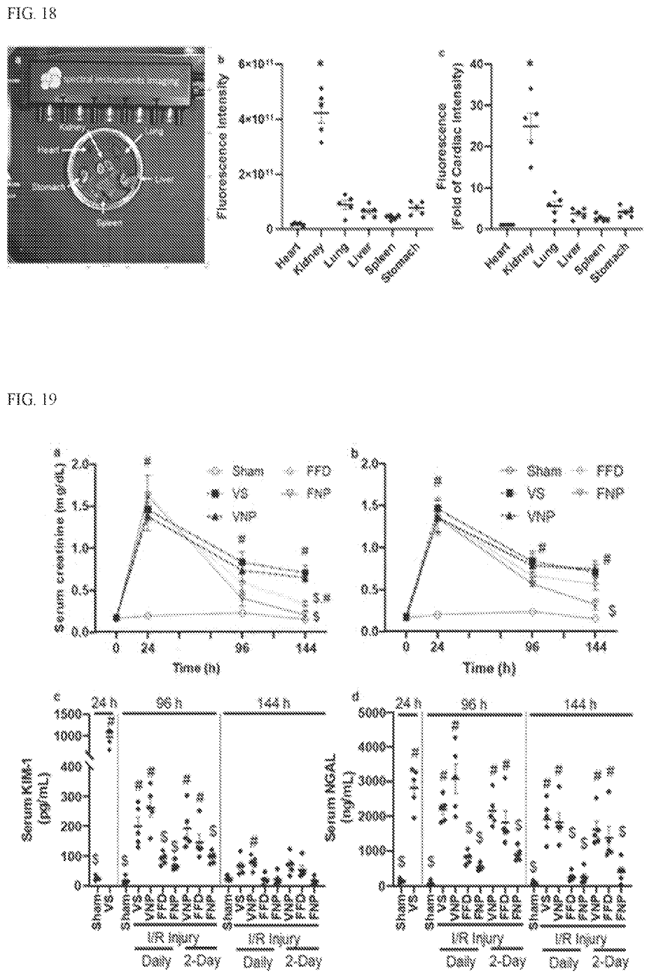

Following 24 hours of reperfusion, mice exhibited serum creatinine concentrations of 1.8±0.2 mg/dL, indicating a significant degree of renal injury. Mice were administered 30 mg/kg DEDC-NP by lateral tail vein injection. After 1 hour, total fluorescence was obtained for the following organs: heart, kidney, lung, liver, spleen and stomach (FIG. 18a). The organ weight normalized fluorescence intensity (FIG. 18b) revealed 5-fold greater fluorescence than the next highest organ, the lungs. When compared to cardiac fluorescence (FIG. 18c), nanoparticles localized to the kidneys with 26-fold greater selectivity, indicating that nanoparticles accumulate in the kidneys even following I/R induced AKI.

Renal Delivery of FNP Enhances Recovery from AKI Induced by I/R Injury.

At 24 hours, mice had serum creatinine levels of 1.4-1.6 mg/dL (FIG. 19a, b) which represents a significant degree of injury and loss of renal function. The means of each group were not statistically different from one another. More sensitive biomarkers of renal injury, KIM-1 and NGAL were also assessed on I/R injured and sham mice at 24-hours post injury. Both serum KIM-1 and NGAL were elevated, 959 pg/mL and 2823 ng/mL respectively, compared to sham (FIG. 19c, d).

Mice were treated with either saline i.p., VNP i.v., formoterol free drug (FFD) (0.3 mg/kg) i.p., or FNP (0.04 mg/kg formoterol) i.v at 24 hours post injury. Mice were treated either daily or once at 24 hours post-injury and again 96 hours post-injury (2-day). The total formoterol dose received in each group is shown in Table 5.

By 96 hours post injury, serum KIM-1 among the VS and VNP treated groups remained elevated (11-15-fold) compared to sham (FIG. 19c). Mice treated daily with FFD or FNP had recovered back to sham levels and were 53% and 66% lower than VS treated mice. However, using the 2-day administered (24 hours and 96 hours) protocol, only the FNP treatment resulted in recovery compared to the VS group (52% decrease). Mice dosed with FFD using the 2-day protocol were not significantly different from VS treated mice and remained 8.5-fold greater than shams.

Additionally, serum NGAL was elevated with VS and VNP treatment (30-43-fold) compared to sham (FIG. 19d). Daily FFD and FNP treatment decreased serum NGAL by 63% and 75% respectively compared to VS. Similarly, 2-day treatment with FNP decreased serum NGAL by 59%. The 2-day FFD treatment group was not significantly different from VS treated mice and remained 26-fold greater than shams. This indicates that daily FFD treatment and both daily and 2-day FNP treatment enhances renal recovery as early as 96-hours post injury.

Following 6 days of daily therapy, mice treated with normal saline or VNP exhibited a decrease in serum creatine compared to 24 hours but remained elevated (5-fold) compared to the sham group (FIG. 19a). Both FFD and FNP treatment groups showed complete recovery of serum creatinine to sham levels, 0.20 mg/dL and 0.33 mg/dL, respectively (FIG. 19a). However, using the 2-day administered (24 hours and 96 hours) protocol, only the FNP treatment resulted in recovery compared to VS and VNP (FIG. 19b). By 144-hours only daily VNP treated mice demonstrated elevated serum KIM-1 (3-fold) compared to shams. Serum NGAL however remained elevated in both VS and VNP treated groups (20-24-fold) compared to sham. Daily FFD and FNP treated mice showed no difference in serum NGAL compared to shams and were both 85% decreased compared to VS treated mice. Similarly, 2-day FNP treatment reduced 144-hour serum NGAL by 78% compared to VS while FFD treatment only recovery by 24%.

FNP therapy enhanced recovery even at reduced dosing frequency, recovering on the 2-day dosing protocol after 6 days to the same serum creatinine as daily FFD therapy, 0.31 and 0.33 mg/dL respectively (FIG. 19a, b) as well as both serum KIM-1 and NGAL. Conversely, mice dosed using the 2-day protocol with FFD showed minimal recovery compared to VS demonstrating that formoterol nanoparticle therapy results in improved renal recovery after AKI.

TABLE 5
Total formoterol dosing across groups (FFD-Formoterol
free drug, FNP-Formoterol containing nanoparticles)

| Treatment | Total Formoterol Dose (by 144 hours) | |
|---|---|---|
| Daily FFD | 1.8 | mg/kg |
| 2-day FFD | 0.6 | mg/kg |
| Daily FNP | 0.24 | mg/kg |
| 2-day FNP | 0.08 | mg/kg |

### Renally Targeted FNP Reduces KIM-1 in the Kidneys

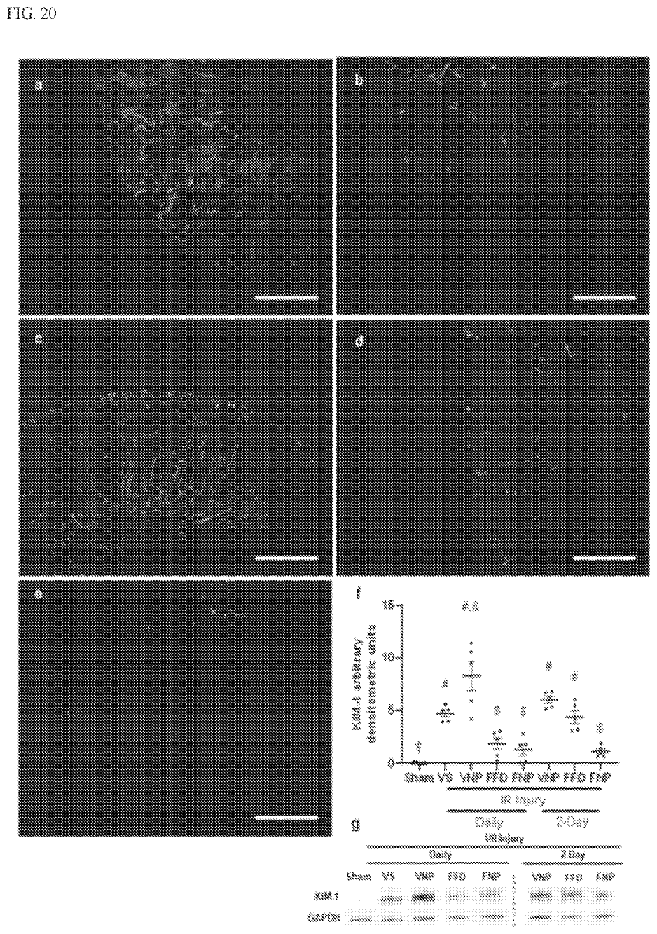

To determine whether FFD and FNP treatment improves tubular injury, the biomarker KIM-1 was evaluated. KIM-1 is a transmembrane protein that is rarely expressed in the healthy kidney but is highly upregulated following acute injury and positively correlates with the extent of kidney damage (Bonventre J V, Yang L. Cellular pathophysiology of ischemic acute kidney injury. J Clin Invest 2011; 121: 4210-4221; Sabbisetti V S, Waikar S S, Antoine D J, et al. Blood kidney injury molecule-1 is a biomarker of acute and chronic kidney injury and predicts progression to ESRD in type I diabetes. J Am Soc Nephrol 2014; 25: 2177-2186). KIM-1 spatial expression pattern was characterized by immunohistochemistry using green fluorescent anti-KIM-1 antibody on renal sections 144 hours following I/R injury. KIM-1 expression is diffuse and present from the renal cortex to the outer stripe of the outer medulla in VS and VNP treated mice (FIG. 20a, b). The intensity and distribution of KIM-1 was markedly decreased with FFD and FNP treatment (FIG. 20c, d). KIM-1 expression was not present to a significant degree in sham controls (FIG. 20e).

Renal Cortex Proteins were Isolated to Quantify KIM-1

I/R injury resulted in an increase in KIM-1 in the cortex of VS animals and was reduced 61% and 73% in daily FFD and FNP treatments respectively (FIG. 20f, g). While 2-day dosing of FFD did not reduce KIM-1 compared to VS, 2-day dosing of FNP showed a decrease in KIM-1 (75% decrease), with no significant difference among daily FFD or daily FNP groups. Mice treated with VNP showed elevated (1.8-fold) KIM-1 compared to VS when dosed daily; however, there was no increase in the 2-day dosing VNP group (1.2-fold difference) compared to VS. Further, VNP given daily to sham mice showed no increase in KIM-1 (1.1-fold difference) and was negligible across all treatment groups (FIG. 30).

### Formoterol Containing Nanoparticles Recover MB Signaling

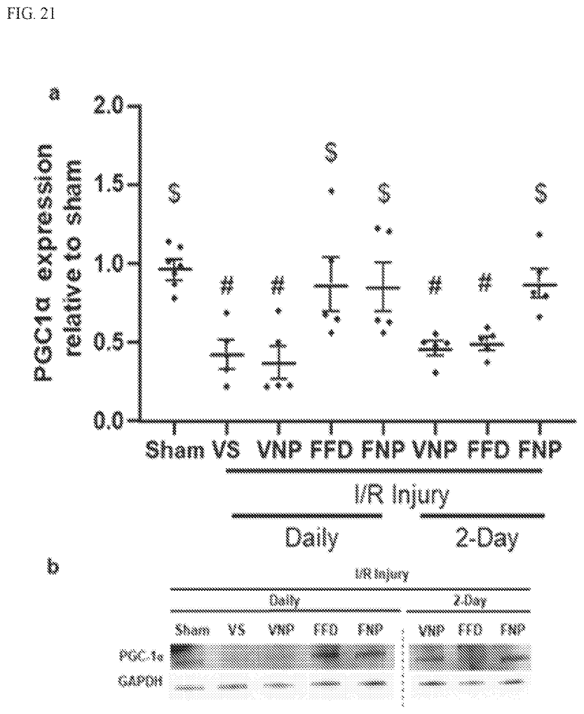

Previous research has shown that mitochondrial function and ETC proteins are reduced following I/R injury in mice (Funk et al., 2012, supra). Further, PGC-1α induces transcription of mitochondrially encoded ETC proteins following I/R injury (Cameron R B, Gibbs W S, Miller S R, et al. Proximal Tubule Beta-2 Adrenergic Receptor Mediates Formoterol-Induced Recovery of Mitochondrial and Renal Function After Ischemia-Reperfusion Injury. J Pharmacol Exp Ther 2019; 369: 173-180). Mice subjected to bilateral I/R injury show a decline in PGC-1α at 144 hours post injury without treatment, 58% and 64% decrease compared to sham for VS and daily VNP groups (FIG. 21). Treatment with daily FFD and FNP showed recovery of PGC-1α to sham levels. In contrast, FFD 2-day dosing did not promote PGC-1α recovery, which remained 52% decreased; whereas with 2-day dosing of FNP, PGC-1α recovered to shams and were not different from either daily FFD or FNP treatment. Taken together, these results indicate that FNP sustains PGC-1α recovery compared to formoterol free drug with 2-day dosing.

### Formoterol Containing Nanoparticles Restore Mitochondrial ETC Protein Level

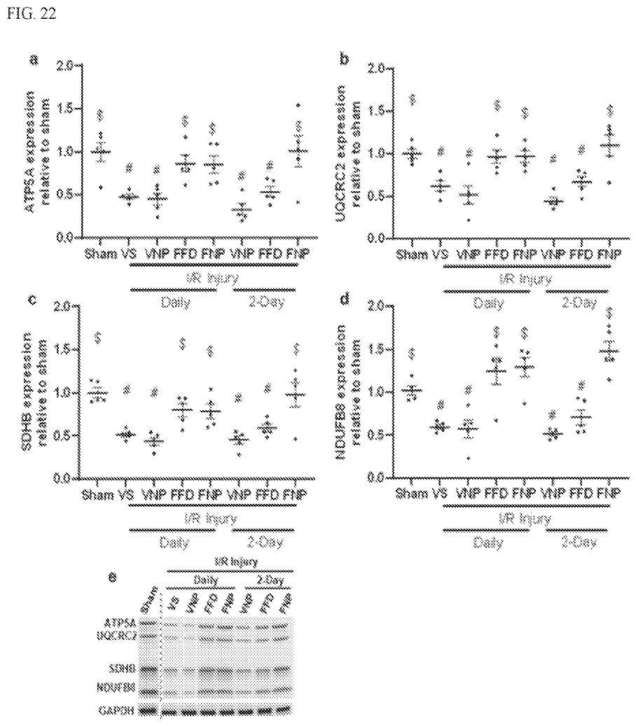

Following AKI, MB suppression results in persistently lowered ETC protein content, inhibiting recovery (Cameron et al., 2019, supra; Tran M, Tam D, Bardia A, et al. PGC-1α promotes recovery after acute kidney injury during systemic inflammation in mice. J Clin Invest 2011; 121: 4003-4014). NDUFB8, SDHB, UQCRC2 and ATP5A are subunits of ETC complexes I, II, III and V respectively. All four complexes were suppressed following AKI, ranging from 38% to 57% decreases for VS and VNP treated mice compared to sham mice (FIG. 22a-d). Daily FFD and FNP restored ETC proteins to their sham levels. Reducing the dosing frequency to 2-days resulted in complete loss of the effects of FFD and were no different from VS. Mice treated with 2-day FNP recovered all four ETC protein subunits back to sham levels.

### Formoterol Containing Nanoparticles Enhance MB Following AKI.

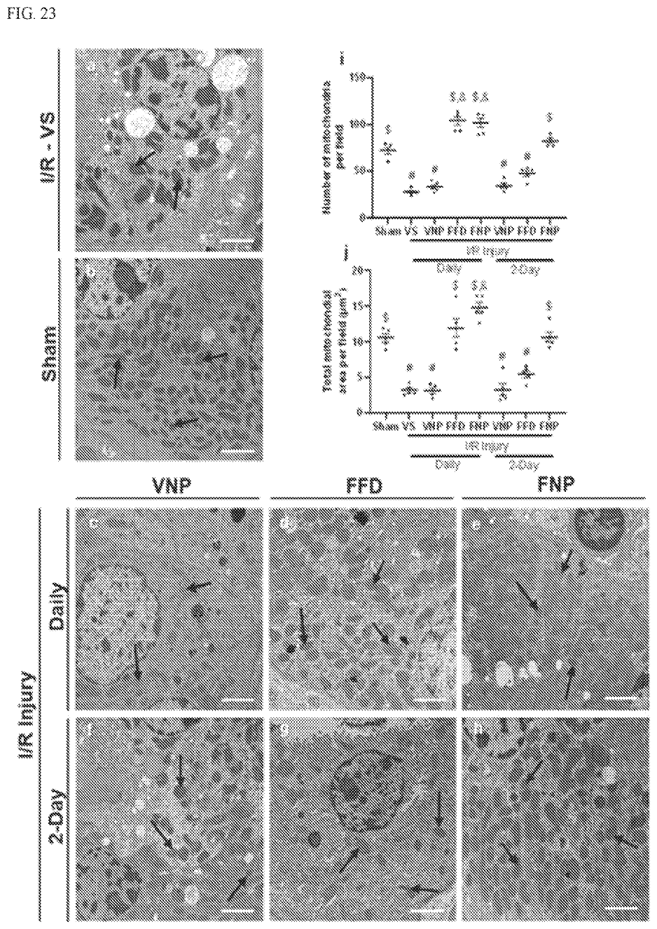

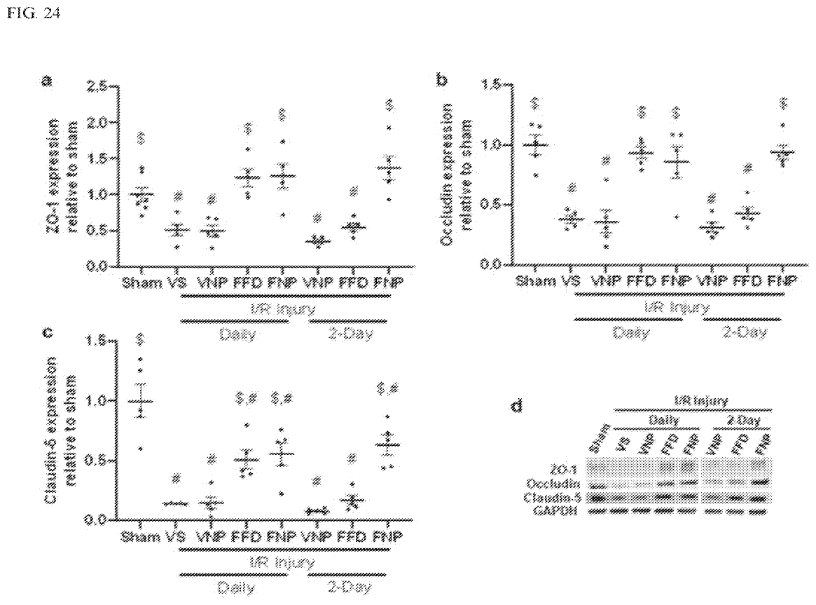

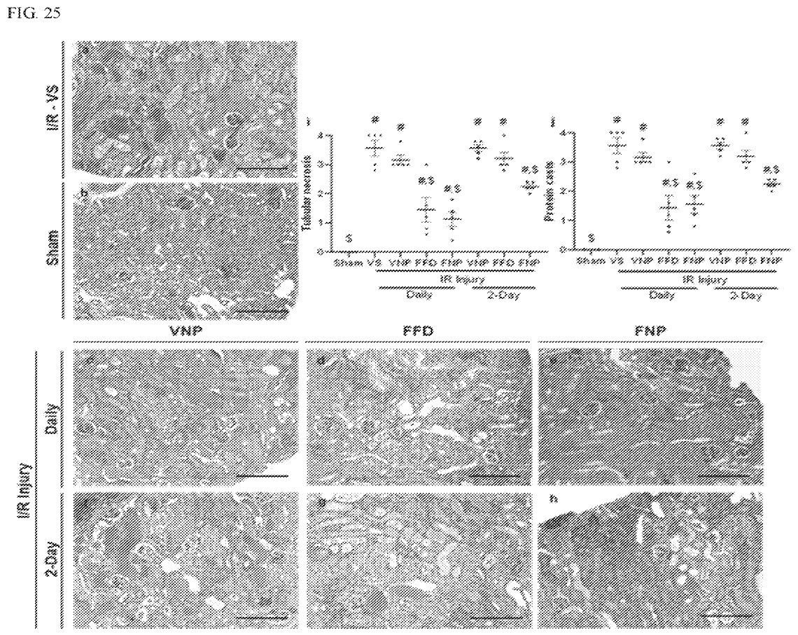

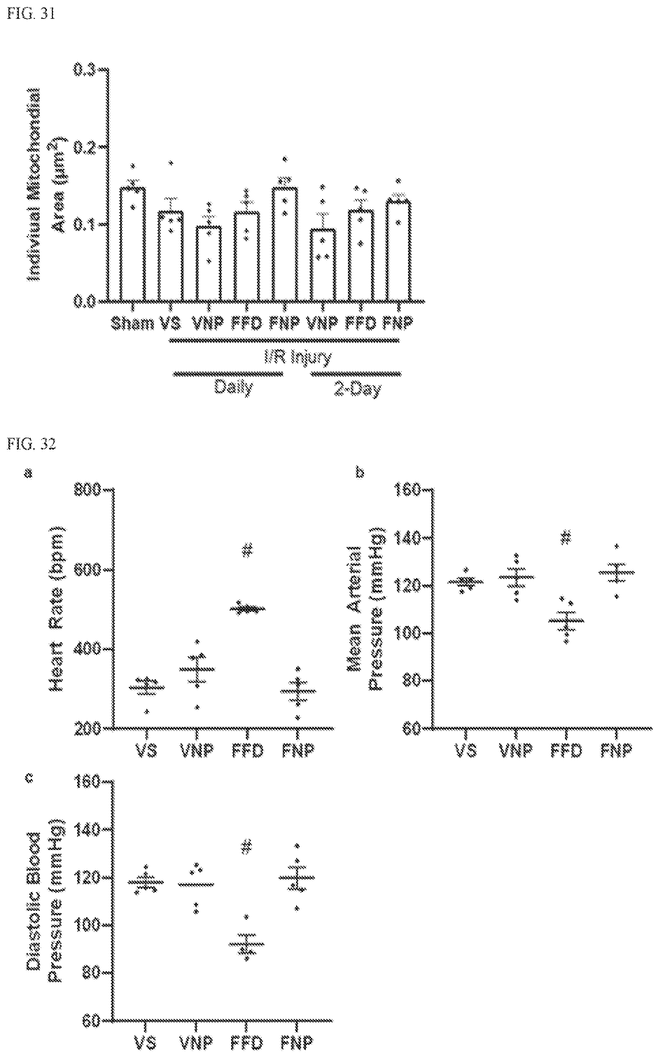

Quantitative analysis of number and size of mitochondria in proximal tubules was carried out 144 hours post I/R injury. Renal cortex from the right superior renal pole was sectioned from each animal and electron micrographs were obtained. There was no difference in individual mitochondrial area, indicating that neither FFD nor FNP induced mitochondrial fission or fusion (FIG. 31). In I/R injured mice treated with VS, a reduction in mitochondrial number, from 72 to 28 mitochondria per field (61% decrease) and total mitochondrial area, from 10.5 μm² to 3.2 μm² per field (70% decrease) compared to sham (FIG. 23i, j). Daily FFD and FNP treatment recovered mitochondrial number above sham (1.4-fold), confirming that formoterol treatment induced MB. 2-day FFD therapy did not completely restore mitochondrial number, which increased to 47 mitochondria per field but was still 35% less than sham. Conversely, 2-day dosed FNP mice showed recovery of mitochondrial number and total area (82 mitochondria and 10.7 μm² per field), indicating that the targeted and sustained renal drug delivery enhances formoterol therapy.

### Formoterol Containing Nanoparticles Recover Epithelial Tight Junction Proteins Following AKI

Mitochondrial dysfunction following I/R injury also results in reduced expression of key epithelial tight junction proteins (Devarajan P. Update on mechanisms of ischemic acute kidney injury. J Am Soc Nephrol 2006; 17: 1503-1520; Kwon O, Nelson W J, Sibley R, et al. Backleak, tight junctions, and cell-cell adhesion in postischemic injury to the renal allograft. J Clin Invest 1998; 101: 2054-2064; Lee D B, Huang E, Ward H J. Tight junction biology and kidney dysfunction. Am J Physiol Renal Physiol 2006; 290: F20-34). Quantitative analysis of tight junction proteins in renal cortex 144 hours post I/R injury revealed decreases in the occludin (62%), claudin-5 (86%) and ZO-1 (50%) in VS treated mice (FIG. 24a-c). Daily treatment with either FFD or FNP recovered ZO-1 and occludin. FFD and FNP also partially recovered claudin-5 expression, however it was still less than sham (50% and 56% of sham) (FIG. 24c). Treatment with FNP on the 2-day protocol saw similar recovery of tight junction proteins; however, 2-day FFD showed no improvement from the VS group (FIG. 24a-c). Restoration of tight junction proteins in the renal cortex with FNP and daily FFD treatment are further evidence of recovering epithelial cells subsequent recovery of renal function.

Proximal Tubule Necrosis and Protein Casts are Reduced with Formoterol Treatment

The effects of formoterol treatment on acute tubular injury after I/R was investigated using PAS-stained sections of the right renal cortex. Compared with sham mice, animals with ischemic AKI exhibited an increase in tubular necrosis, as characterized by the loss of brush border and tubular dilation and proteinaceous cast formation in the lumen of tubules at 144-hours post injury (FIG. 25i, j). Mice treated daily with FFD or FNP showed a 60% and 69% reduction in tubular necrosis compared to VS. When FFD dosing frequency was reduced to 2-day, mice showed no improvement in scored tubular necrosis (3.2) or protein casts (3.2) compared to VS (both 3.6). Conversely, 2-day FNP treatment still showed recovery, a 38% decrease in tubular necrosis compared to VS and VNP. While there was significant recovery, the 2-day dosing of FNP did not recover to the same extent as daily dosing FNP. However, there was no significant difference between 2-day dosing FNP and daily FFD therapies.

### Formoterol Treatment Protects Against Renal Fibrosis Following I/R Injury

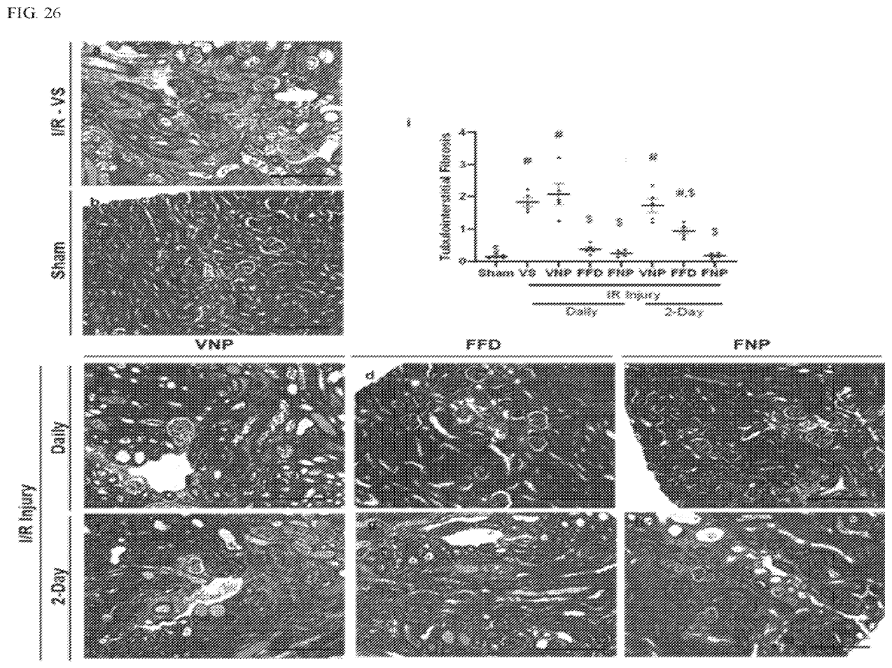

Renal fibrosis is a common result of unrecovered AKI and a hallmark of the acute to chronic injury transition. Mice treated with VS or VNP showed increased (11-14-fold) renal fibrosis compared to shams by 144 hours post I/R injury (FIG. 26i). Daily FFD and FNP therapy protected against fibrosis, decreasing trichrome collagen staining by 79% and 86% respectively compared to VS treated mice. Treatment following the 2-day dosing protocol with FNP showed a similar, 90% reduction in fibrosis compared to VS treated mice. Conversely 2-day FFD therapy only resulted in a 48% reduction in fibrosis which is an improvement from VS treated mice but also exhibits significantly (6.3-fold) greater collagen staining than shams.

### Formoterol Containing Nanoparticles Prevent Acute Cardiovascular Effects

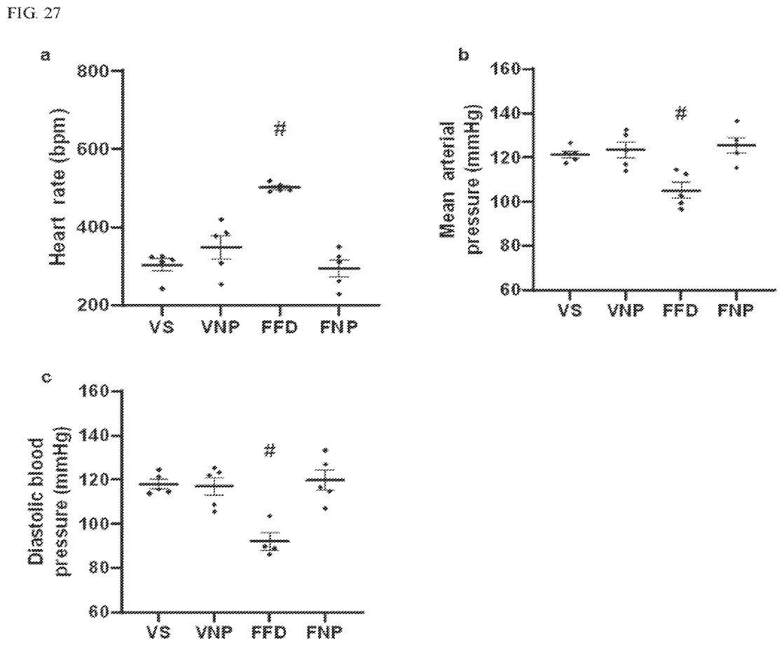

Systemic formoterol administration has been shown to cause alterations in cardiac function, specifically diastolic dysfunction and tachycardia (Guhan A R, Cooper S, Oborne J, et al. Systemic effects of formoterol and salmeterol: a dose-response comparison in healthy subjects. Thorax 2000; 55: 650-656; Bernstein D, Fajardo G, Zhao M. The Role of B-Adrenergic Receptors in Heart Failure: Differential Regulation of Cardiotoxicity and Cardioprotection. Prog Pediatr Cardiol 2011; 31: 35-38; Brouri F, Findji L, Mediani O, et al. Toxic cardiac effects of catecholamines: role of beta-adrenoceptor downregulation. Eur J Pharmacol 2002; 456: 69-75; Faubel S, Shah P B. Immediate Consequences of Acute Kidney Injury: The Impact of Traditional and Nontraditional Complications on Mortality in Acute Kidney Injury. Adv Chronic Kidney Dis 2016; 23: 179-185). Mice subjected to I/R injury were dosed with VS, VNP, FFD or FNP and their heart rate and blood pressure assessed 1-2 hours following administration of therapy. Mice received FFD exhibited profound tachycardia, a 1.5-fold increase from VS (FIG. 27a). Mice dosed with FNP showed no change in heart rate. Similarly, mice administered FFD showed an 18% decrease in mean arterial pressure (MAP), (FIG. 27b, c). FNP had no effect on heart rate, MAP and diastolic blood pressure compared to VS (<1 mmHg difference). These cardiovascular changes were similarly observed in mice without I/R injury (FIG. 32). Taken together, these results reveal that renally targeted nanoparticle delivery ameliorates the tachycardic and hypotensive effects of formoterol.

### Formoterol Nanoparticle Treatment Protects Against MB-Induced Cardiac Hypertrophy

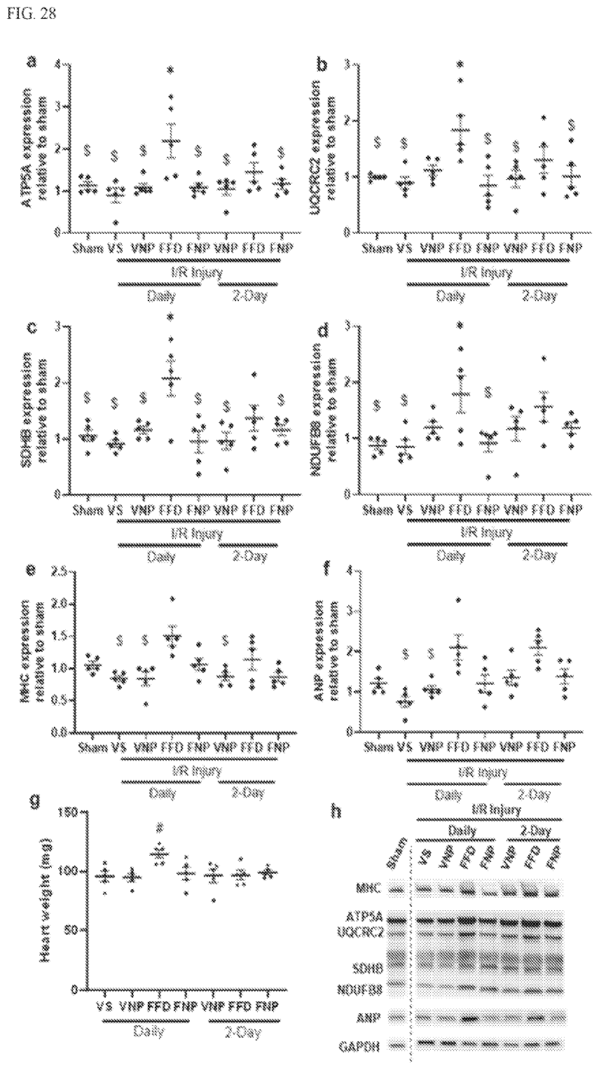

Repeated administration of β-adrenergic receptor agonists has been shown to result in muscle hypertrophy (Bernstein et al., 2011, supra; Brouri et al., 2002, supra; Faubel et al., 2016, supra). In mice treated with FFD, left ventricular ETC protein expression is elevated compared to VS treated mice (FIG. 28a-d). ETC protein subunits NDUFB8, SDHB, UQCRC2 and ATP5A were shown to be increased 2.0, 2.0, 1.8 and 1.9-fold respectively with daily FFD treatment and 1.8, 1.3, 1.3 and 1.2-fold with the 2-day FFD treatment. Conversely, renally targeted nanoparticle therapy with FNP resulted in no increase in the same proteins with either dosing regimen, suggesting limited induction of MB in the heart. Additionally, daily and 2-day FFD treatment resulted in increased myosin heavy chain (MHC) (1.4 and 1.1-fold respectively) and atrial natriuretic peptide (ANP) (1.7-fold for both), which are biomarkers of cardiac hypertrophy (FIG. 28e, f). Treatment with FNP exhibited no increase in either marker with daily or 2-day dosing protocols. Formoterol-induced increase in cardiac mass was also assessed. Mice treated daily with FFD showed a slight increase in cardiac mass, 114 mg and 96 mg for FFD and VS, respectively (FIG. 28g). FNP treated mice showed no significant increase in cardiac mass.

### Example 4

### Methods

Poly(lactide-co-glycolide) methyl ether block-poly(ethylene glycol)-amine (PLGA-PEG-HN₂) PLGA average Mₙ 20,000, PEG average Mₙ 5,000 were purchased from Nanosoft Polymers (Winston-Salem, NC). Acetic acid (HPLC grade) was purchased from ThermoFisher (Waltham, MA). Formoterol fumarate dehydrate, 420.46 MW, >98% pure, hydrochloric acid 1 N (ACS grade), poly(vinyl alcohol) (PVA) (89,000-98,000 MW, >99% hydrolyzed, reagent grade), Sodium choate hydrate (>99% purity), potassium phosphate monobasic (>99% purity), potassium phosphate dibasic (>98% purity), formic acid (97.5-98.5% purity), neutral buffered formalin solution (10%, histology grade), Triton™ X-100 (molecular biology grade), sodium fluoride (≥99% purity), sodium orthovanadate (>90% purity), protease inhibitor cocktail (molecular biology grade), hydrogen peroxide (3%, molecular biology grade), Tween® 20 (molecular biology grade) and resazurin sodium salt (˜80% dye content, molecular biology grade) were purchased from Sigma-Aldrich (St. Louis, MO). (+)-Trehalose dihydrate (387.32 g/mol) and 3,3′-diethylthiadicarbocyanine iodide was purchased from Acros Organics (Fair Lawn, NJ, USA). Choloroform (ACS grade), anhydrous acetonitrile (LCMS grade), and methanol (LCMS grade) were purchased from Spectrum Chemical Mfg. Corp. (Gardena, CA). Collagen I, phospo-SMAD3, SMAD3, TGFβ, and GAPDH primary antibodies, secondary antibodies, KIM-1 and NGAL ELISA kits were purchased from abcam (Cambridge, MA). Creatinine enzymatic reagent assay kit was purchased from Diazyme Laboratories, Inc. (La Jolla, CA). Albuwell M: murine microalbuminuria ELISA was purchased from Ethos Biosciences (Newton Square, PA). Fluorescein-isothiocyanate-labelled sinistrin (>99% pure) was purchased from Medibeacon GmBH Mannheim, Germany).

### Synthesis of PLGA-PEG-NH₂ Nanoparticles

Poly(lactide-co-glycolide) methyl ether block-poly(ethylene glycol)-amine (PLGA-PEG-HN₂) (Sigma-Aldrich, St. Louis, MO) nanoparticles containing either vehicle (Veh(NP)), formoterol (FFD(NP)), 3,3′-diethylthiadicarbocyanine iodide (DEDC) (DEDC(NP)) were synthesized using the water-in-oil-in-water double-emulsion solvent evaporation method. Briefly, 20 mg of PLGA-PEG-HN₂ was dissolved in chloroform. Formoterol fumarate dihydrate (Sigma-Aldrich, St. Louis, MO) or 3,3′-diethylthiadicarbocyanine iodide (DEDC) (Acros Organics; Geel, Belgium) was dissolved in 10 mM sodium cholate and was added in a 1:2 aqueous:organic ratio. The primary emulsion was then formed using a microtip probe sonicator (Qsonica, Newtown, CT) at 60 Watt of energy output for 30 seconds over an ice bath. The primary emulsion was then diluted with 1% PVA (Sigma-Aldrich, St. Louis, MO) and sonicated for 3 min at 60 Watt over an ice bath. The nanoparticles were then allowed to harden and organic solvent evaporate with stirring at room temperatures. The nanoparticle suspension was centrifuged at 15,000 relative centrifugal force (rcf) and washed thrice with distilled ultrapure water (18.2 MΩ·cm) (Milli-Q Plus, MilliporeSigma, Burlington, MA). Samples were lyophilized at −80° C. under a vacuum <0.133 mmHg (FreeZone 4.5 L, Labconco, Kansas City, MO) in 5% α-Trehalose (Acros Organics) and stored at −20° C. until used in the physical characterization and in vivo animal studies.

### Characterization of PLGA-PEG-NH₂ Nanoparticles

An HPLC system (Alliance 2965, Waters, Milford, MA) with a UV dual-wavelength detector (Alliance 2487 Dual Wavelength Absorbance Detector, Waters, Milford, MA) with a C18-column (4.6 mm×250 mm length, 5 μm pore size) (Phenomenex Torrance, CA) was used to quantify the amount of formoterol drug encapsulated within the polymeric nanoparticles and the amount released. The mobile phase consisted of methanol (Spectrum Chemical MFG Corp., Gardena, CA) and 50 mM phosphoric acid (Sigma-Aldrich, St. Louis, MO) buffer with 1% acetic acid (ThermoFisher, Waltham, MA) at a ratio of 65:35, 1.0 mL/min flow rate and a column temperature of 40° C. The detection wavelength was 242 nm and the injection volume was 10 μL. The % drug loading (DL) was calculated, as previously reported (S. Faubel and P. B. Shah, “Immediate Consequences of Acute Kidney Injury: The Impact of Traditional and Nontraditional Complications on Mortality in Acute Kidney Injury,” (in eng), Adv Chronic Kidney Dis, vol. 23, no. 3, pp. 179-85, May 2016), using Equation 1:

$$DL ( \% ) = \frac{the amount of formoterol assayed}{the total amount of nanoparticles in the preparation} \times 100 \qquad Equation 1$$

The % encapsulation (entrapment) efficiency (EE) was calculated, as previously reported (S. Zhou, W. Sun, Z. Zhang, and Y. Zheng, “The role of Nrf2-mediated pathway in cardiac remodeling and heart failure,” (in eng), Oxid Med Cell Longev, vol. 2014, p. 260429, 2014) using Equation 2:

$$EE ( \% ) = \frac{the amount of formoterol assayed}{the total amount of formoterol used in the preparation} \times 100 \qquad Equation 2$$

Hydrodynamic particle size distribution of the nanoparticles was determined by photon correlation spectroscopy using the Zetasizer Nano ZS (Malvern Instruments Ltd., Malvern, UK), using similar conditions and procedures published earlier (Zhou et al., 2014, supra). The nanoparticle suspension was evaluated using a scattering angle of 173° at a temperature of 25° C. A minimum of 10 measurements were taken per replicate and samples were evaluated a minimum of 3 replicates (n≥3).

Zeta potential (ζ) measurements were carried out in 0.1× normal saline solution at 25° C. and pH 7.2. The mean ζ was determined using the Zetasizer Nano ZS (Malvern Instruments Ltd., Malvern, UK) phase analysis light scattering technique.

Using similar conditions as previously reported (M. St John Sutton et al., “Left ventricular remodeling and ventricular arrhythmias after myocardial infarction,” (in eng), Circulation, vol. 107, no. 20, pp. 2577-82, May 2003), FFD(NP)s were dispersed in 10 mL of phosphate buffered saline (PBS) producing a final pH 7.4 and incubated at 37° C. under magnetic stirring. At determined time intervals up to 144 h, aliquots were centrifuged at 10,000 rcf for 10 min. Supernatant was extracted and replaced with equal volume fresh PBS in order to maintain sink conditions. Samples were analyzed by HPLC, as described above. Nanoparticle release was determined in triplicate (n=3).

### Animal Use

Mice and rabbits were housed in the University of Arizona Animal Care (UAC) Facility at a constant temperature of 22±2° C. with 12:12 h light-dark cycles and received standard rodent food and water ad libitum.

### Renal Proximal Tubule Cell Biocompatibility and Uptake

RPTC were isolated from female New Zealand white rabbits (Crl:KBL(NZW)) purchased from Charles River (Oakwood, MI) as previously described using the iron oxide perfusion method (G. Nowak and R. G. Schnellmann, “Improved culture conditions stimulate gluconeogenesis in primary cultures of renal proximal tubule cells,” (in eng), Am J Physiol, vol. 268, no. 4 Pt 1, pp. C1053-61, April 1995). RPTCs were grown to confluence with 1:1 DMEM:F-12 (without glucose, phenol red, or sodium pyruvate) media supplemented with 15 mM HEPES buffer, 2.5 mM L-glutamine, 1 μM pyridoxine HCL, 15 mM sodium bicarbonate, and 6 mM lactate (ThermoFisher). Hydrocortisone (50 nM, Sigma-Aldrich, St. Louis, MO), selenium (5 ng/mL, Sigma-Aldrich), human transferrin (5 μg/mL, Sigma-Aldrich), bovine insulin (10 nM, Sigma-Aldrich), and L-ascorbic acid-2-phosphate (50 μM, ThermoFisher) were added daily to fresh culture medium.

To assess the toxicity of FFD(NP)s, RPTCs were seeded in each well of a 96-well plate (5×10³ cells/well) and grown to confluence. Different concentrations of FFD(NP)s were added to each well and incubated for 24 h. Toxicity was assessed by resazurin reduction assay, as described previously (M. F. Acosta, M. D. Abrahamson, D. Encinas-Basurto, J. R. Fineman, S. M. Black, and H. M. Mansour, “Inhalable Nanoparticles/Microparticles of an AMPK and Nrf2 Activator for Targeted Pulmonary Drug Delivery as Dry Powder Inhalers,” (in eng), AAPS J, vol. 23, no. 1, p. 2, November 2020; W. Alabsi, F. A. Al-Obeidi, R. Polt, and H. M. Mansour, “ Organic Solution Advanced Spray-Dried Microparticulate/Nanoparticulate Dry Powders of Lactomorphin for Respiratory Delivery: Physicochemical Characterization, In Vitro Aerosol Dispersion, and Cellular Studies.,” Pharmaceutics, vol. 13, p. Article 26, 2021) (n=5 biological replicates). Detection was measured using Synergy H1 Multi-Mode Reader (BioTek Instruments, Inc., Winooski, VT) with 544 nm excitation and 590 nm emission wavelengths. Five biological replicates were used and viability of the cells was calculated as follows:

$$Relative viability ( \% ) = \frac{Sample fluorescence intensity}{Control fluorescence intensity} \times 100 \% \qquad Equation 3$$

To assess uptake of DEDC(NP)s, RPTCs were grown in 35-mm tissue culture dishes as described above. DEDC(NP) dispersions were prepared by diluting nanoparticles into media and vortexed immediately prior to experiments. Cells were preincubated for 30 min with varying concentrations of either media or the drugs chlorpromazine hydrochloride (Sigma-Aldrich), nocodazole (Sigma-Aldrich), or simvastatin (Sigma-Aldrich). After pre-incubation, DEDC(NP)s were added at a final concentration of 0.1 mg/mL and incubated for 2 h at 37° C. Cells were washed in triplicate with media to ensure removal of nanoparticles adhered to the outer cell membrane. Cells were then fixed in 10% neutral buffered formalin and nuclei stained with 4′,6-diamidino-2-phenylindole (DAPI). Cells were imaged using the EVOS M5000 Imaging System (ThermoFisher, Waltham, MA) and fluorescence intensity was analyzed by ImageJ (NIH) (n=5 biological replicates).

### Renal Localization of Nanoparticles

The renal localization of DEDC(NP)s was assessed in eight-week-old male SKH-1 Elite hairless mice (Crl:SKH1-Hr^{hr}) (Charles River, Troy, NY). Groups of n=5 mice were each administered 30 mg/kg DEDC(NP)s either intravenously via lateral tail vein injection, intraperitoneal injection, subcutaneous injection or oral gavage. A control group of n=5 mice was administered 100 μL of 25 μg/kg DEDC in normal saline as described previously. Dorsal images of mice were acquired using the Lago live animal fluorescent imaging system (Spectral Instruments Imaging, Tucson, AZ) with 675 nm excitation and 710 nm emission filters. Images were taken at 1, 3, 12, 24, 72, 120 and 144 h following nanoparticle or dye administration. Additional mice (n=5) were administered 30 mg/kg of DEDC(NP)s and euthanized 3 h following administration. The kidneys, heart, lung, liver, pancreas and spleen were removed, weighed and imaged for fluorescence. Analysis of florescence images was carried out using Aura imaging software (Spectral Instruments Imaging, Tucson, AZ) with equal sized regions of interest (ROI) selected around each kidney in live animals to obtain the total fluorescence emission (TFE). In ex-vivo organ analysis, TFE for each organ was normalized to organ weight.

### Nanoparticle Dose Selection

The FFD(NP) dosage and frequency was determined using BTBR wild type mice (BTBR T⁺ Itpr3^{tf}/J) (The Jackson Laboratory, Bar Harbor, ME). Mice were divided into seven groups to be dosed for seven days (Table 6). The Veh(Sal) group was given intraperitoneal injections of normal saline daily. The Veh(NP) group was given 60 mg/kg nanoparticles injected via lateral tail vein 2× per week. The FFD(NP) groups were given 30 mg/kg or 60 mg/kg nanoparticles (0.5 or 1 mg/kg formoterol) injected via lateral tail, either once or twice per week. The FFD(Sal) group was given intraperitoneal injections of 1 mg/kg formoterol in normal saline daily, this dose was selected after it showed success at reducing hyperglycemia induced mitochondrial dysfunction in a diabetic mouse model (N. B. Flemming, L. A. Gallo, M. S. Ward, and J. M. Forbes, “Tapping into Mitochondria to Find Novel Targets for Diabetes Complications,” (in eng), Curr Drug Targets, vol. 17, no. 12, pp. 1341-9, 2016). After eight days, mice were weighed, euthanized and kidneys were extracted and snap-frozen in liquid nitrogen.

TABLE 6
Total Weekly Formoterol Exposure

| 7-day dosing | Total Weekly Dose | |
|---|---|---|
| Daily Formoterol IP (1.0 mg/kg) | 7 | mg/kg |
| 2/week Formoterol NP (60 mg/kg) | 2 | mg/kg |
| 1/week Formoterol NP (60 mg/kg) | 1 | mg/kg |
| 2/week Formoterol NP (30 mg/kg) | 1 | mg/kg |
| 1/week Formoterol NP (30 mg/kg) | 0.5 | mg/kg |

### Nanoparticle Treatment of Diabetic Mice

The efficacy of nanoparticle therapy was determined using BTBR wild type and obese diabetic mice (BTBR T⁺ Itpr3^{tf}/J (WT) and BTBR.Cg-Lep^{ob}/WiscJ (ob/ob)) (The Jackson Laboratory, Bar Harbor, ME). Five-week-old male WT and ob/ob mice were randomly assigned into eight groups (n=8-16 per group): vehicle saline (Veh(Sal)), vehicle nanoparticle (Veh(NP)), formoterol saline (FFD(Sal)), or formoterol nanoparticle (FFD(NP)) for both WT and obese diabetic animals. Mice in each of the nanoparticle groups, Veh(NP) and FFD(NP), received weekly 30 mg/kg nanoparticle (0.5 mg/kg formoterol) intravenous injections via the lateral tail vein and equal volumes of normal saline on all other days. Mice in the formoterol groups, FFD(Sal), received daily 1 mg/kg intraperitoneal injections of formoterol in normal saline. Mice in the vehicle saline groups, Veh(Sal), received daily intraperitoneal injections of normal saline. Immediately following study initiation, 8 mice from both WT and ob/ob Veh(Sal) were euthanized and kidneys extracted for histological analysis. Remaining mice (n=8 per group) were treated for 8 weeks. Following the final week of treatment, mice were sacrificed and kidneys were perfused with normal saline with heparin (10 U/mL). Serum, kidneys and heart were collected for protein and histological analysis.

### Blood Glucose Measurement

At 5, 8 and 12 weeks of age blood glucose was measured following 6 h of fasting using the AlhaTRAK2 blood glucose monitor (Zoetis, Parsippany-Troy Hills, NJ) using the supplier recommended diabetic mouse model codes (P. Molenaar, L. Chen, and W. A. Parsonage, “Cardiac implications for the use of beta2-adrenoceptor agonists for the management of muscle wasting,” (in eng), Br J Pharmacol, vol. 147, no. 6, pp. 583-6, March 2006; N. E. Scholpa, E. C. Simmons, J. D. Crossman, and R. G. Schnellmann, “Time-to-treatment window and cross-sex potential of Beta 2-adrenergic receptor-induced mitochondrial biogenesis-mediated recovery after spinal cord injury,” (in eng), Toxicol Appl Pharmacol, vol. 411, p. 115366, January 2021).

### Urinalysis

Urine samples were collected by metabolic caging for 18 h at 5, 8 and 12 weeks of age. The urinary albumin concentration was measured by Albuwell M mouse albumin ELISA kit (Ethos Biosciences, Newtown Square, PA). Urinary creatinine was quantified using the Creatinine Enzymatic Reagent Assay kit (Diazyme Laboratories, Inc. La Jolla, CA), according to the manufacturer's protocol. Urinary KIM-1 and NGAL was quantified using mouse KIM-1 and NGAL ELISA kits (Abcam, Cambridge, UK) according to the manufacturer's instructions.

### Glomerular Filtration Rate

The glomerular filtration rate (GFR) was determined at 5, 8 and 12 weeks of age. GFR was measured using a transdermal GFR monitor (Medibeacon GmBH, Mannheim, Germany) as previously described (L. Scarfe et al., “Transdermal Measurement of Glomerular Filtration Rate in Mice,” (in eng), J Vis Exp, no. 140, Oct. 21, 2018). Briefly, FITC-sinistrin (Medibeacon GmBH) was dissolved in PBS at a concentration of 15 mg/mL. Mice were anesthetized by isoflurane inhalation, the GFR monitor attached, and injected with 7.5 mg/100 g BW of FITC-sinistrin by retro-orbital injection. Mice were recovered from sedation and elimination was measured for 1.5 h. GFR was calculated using MB Studio (Medibeacon GmBH) using a 3-compartment model with linear correction as previously reported (Scarfe et al., 2018, supra).

### Histological Analysis

Perfused organs were fixed in 10% neutral formalin buffer for 24 h and embedded in paraffin and sectioned at 4 μm. Staining and immunohistochemistry was performed on a Bond Rx autostainer (Leica Biosystems, Wetzlar, Germany). Whole slide scanning was performed using an Aperio AT2 (Leica Biosystems). Antibodies used were rat monoclonal F4/80 primary antibody (ThermoFisher). Randomization of image selection was carried out by overlaying a grid on whole slide and assigning random numbers to each grind square from which images were taken in order. All quantification was carried out using ImageJ. Mesangial matrix expansion was determined by measuring the PAS positive, nuclei-free area as a fraction of the glomerular tuft as previously described (L. Wu et al., “Annexin A1 alleviates kidney injury by promoting the resolution of inflammation in diabetic nephropathy,” (in eng), Kidney Int, vol. 100, no. 1, pp. 107-121, July 2021), at least 20 glomeruli were measured per animal. Glomerular size was measured by determining the cross-sectional area of the glomerular tuft in PAS-stained images, at least 30 glomeruli were measured per animal. Glomerular and interstitial collagen deposition was determined by quantifying the fraction of picrosirius red-positive stain at 20× magnification, at least 20 interstitial regions restricted to the kidney cortex or glomeruli were measured per animal. Macrophage infiltration was measured by quantifying the number of F4/80-positive cells in each of 20 400× magnification fields restricted to the kidney cortex per animal.

Cardiovascular fibrosis was measured by quantification of the fraction of trichrome-positive area in both interstitial and perivascular sections of the right ventricular wall. At least 15 sections were quantified per animal. All quantification was completed by an analyst blinded to treatment group.

### Protein Isolation and Immunoblotting

Protein was extracted from renal cortical tissue using radioimmunoprecipitation assay (RIPA) buffer (50 mM Tris-HCl, 150 mM NaCl, 0.1% SDS, 0.5% sodium deoxycholate and 1% Triton X-100, pH 7.4) with protease inhibitor cocktail (1:100), 1 mM sodium fluoride, and 1 mM sodium orthovanadate (Sigma Aldrich). Equal protein quantities (10 μg) were loaded onto 4-15% SDS-PAGE gels, resolved by gel electrophoresis, and then transferred onto nitrocellulose membranes. Membranes were blocked in 5% milk in tris-buffered saline/tween 20, incubated at a temperature of 4° C. for 18-24 h with primary and secondary antibodies (Abcam). Bound antibodies were visualized using enhanced chemiluminescence (Thermo Scientific, Waltham, MA) and the GE ImageQuant LAS400 Biomolecular Imaging System (GE Healthcare, Marlborough, MA).

### Statistical Analysis

Data are expressed as mean±S.E.M. for all experiments. Statistical significance for two groups was determined via students t-test. For multiple groups one- or two-way ANOVA was used and differences between groups was determined using Tukey post hoc test. The criteria for statistical significance were P<0.05 for all experiments.

### Results

### Physicochemical Characterization of Polymeric Nanoparticles

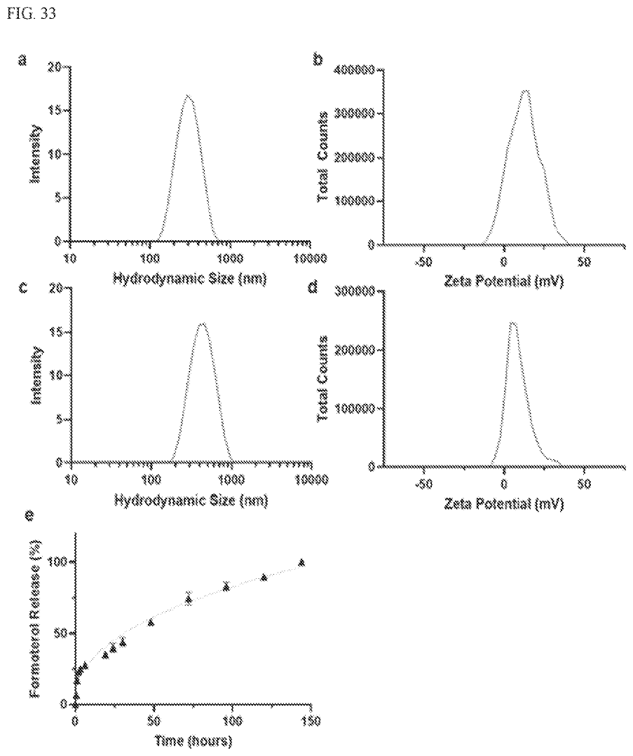

The DEDC(NP) and FFD(NP)s were prepared using diblock copolymer of PLGA-PEG-NH₂ as previously described (E. L. Vallorz, K. Blohm-Mangone, R. G. Schnellmann, and H. M. Mansour, “Formoterol PLGA-PEG Nanoparticles Induce Mitochondrial Biogenesis in Renal Proximal Tubules,” (in eng), AAPS J, vol. 23, no. 4, p. 88, June 2021). The double emulsion solvent evaporation method was used to formulate both nanoparticles. Particle size was confirmed to be 328±12 nm and 388±27 nm for the DEDC(NP) and FFD(NP) respectively) which is within the range previously shown to be effective at localizing to the kidneys (Vallorz et al., supra; Zhang and S. S. Feng, “The drug encapsulation efficiency, in vitro drug release, cellular uptake and cytotoxicity of paclitaxel-loaded poly(lactide)-tocopheryl polyethylene glycol succinate nanoparticles,” (in eng), Biomaterials, vol. 27, no. 21, pp. 4025-33, July 2006). Particle effective zeta potential, a measure of surface charge, was determined to be 12.1±2.3 mV and 5.7±1.7 mV for the DEDC(NP) and FFD(NP) respectively (FIG. 33b, d). The FFD(NP)s contained 0.017 mg of formoterol per mg of nanoparticle, a drug loading of 1.7% which represents an encapsulation efficiency of 70%. Over 90% of the entrapped drug was released over the 144-h studied. With ˜25% released within the initial burst phase and the remainder released at a constant rate (FIG. 33e).

### Nanoparticles are Biocompatible and Endocytosed by Renal Proximal Tubule Cells

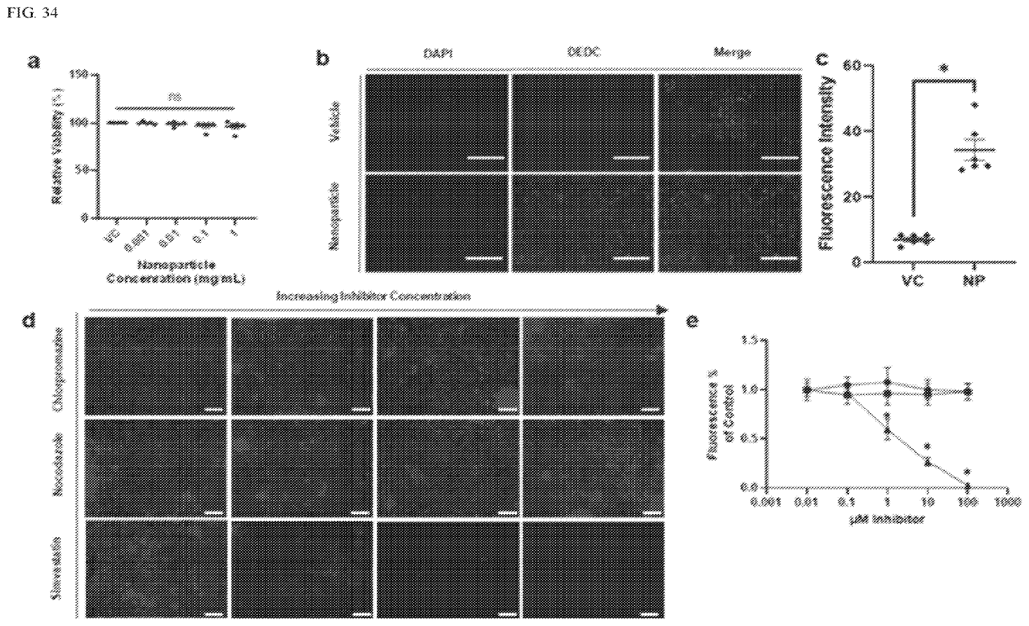

Previous studies have shown that formoterol is non-toxic to RPTCs up to a concentration of 1000 nM (Vallorz et al., supra). Here, FFD(NP)s at concentrations ranging from 0.001 to 1 mg/mL were exposed to RPTCs for 24 h and relative viability determined. Across the range evaluated, no concentration of FFD(NP)s resulted in decline in RPTC viability (FIG. 34a). Uptake of the PLGA-PEG-NH₂ nanoparticles was determined using DEDC(NP)s which contain the fluorescent dye DEDC. RPTCs were exposed to either 0.1 mg/mL of DEDC(NP)s or an equivalent amount of free dye and incubated with stirring for 2 h. After incubation and washing, intracellular fluorescence is strongly visible in DEDC(NP) exposed RPTCs and minimal in free dye treated cells which is confirmed with quantitative fluorescent analysis (FIG. 34b, c).

Pretreatment of RPTCs with endocytosis inhibitors chlorpromazine, nocodazole, and simvastatin followed by DEDC(NP) exposure showed a concentration dependent decrease in nanoparticle uptake with simvastatin pretreatment, starting at 1 μM inhibitor and resulting in complete reduction in intracellular fluorescence intensity at 100 μM (FIG. 34d, e). There was no observable decline in uptake with either chlorpromazine or nocodazole pretreatment. These results suggest that DEDC(NP)s are not endocytosed via a clathrin-mediated mechanism or micropinocytosis, which would be inhibited by chlorpromazine and nocodazole, rather, uptake may be dependent on caveolae-mediated or lipid vesicle dependent mechanisms (V. Francia, C. Reker-Smit, G. Boel, and A. Salvati, “Limits and challenges in using transport inhibitors to characterize how nano-sized drug carriers enter cells,” (in eng), Nanomedicine (Lond), vol. 14, no. 12, pp. 1533-1549, June 2019; T.-G. Iversen, T. Skotland, and K. Sandvig, “Endocytosis and intracellular transport of nanoparticles: Present knowledge and need for future studies,” Nano Today, vol. 6, no. 2, pp. 176-185, 2011; J. P. Caviston and E. L. Holzbaur, “Microtubule motors at the intersection of trafficking and transport,” (in eng), Trends Cell Biol, vol. 16, no. 10, pp. 530-7, October 2006).

### Nanoparticles Localize to the Kidneys of Mice in a Route of Administration Dependent Manner

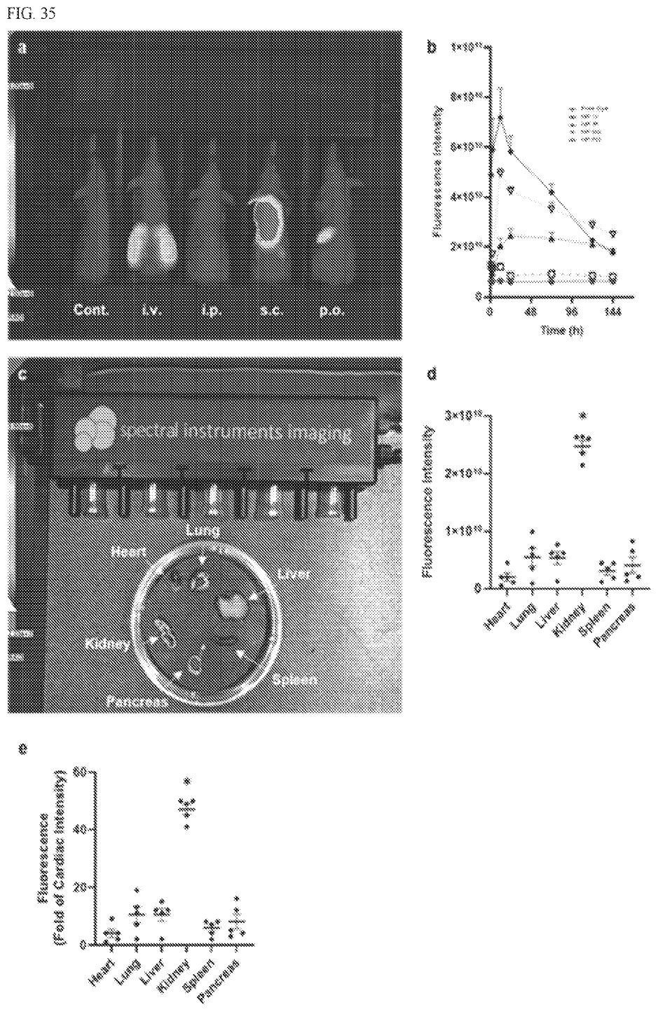

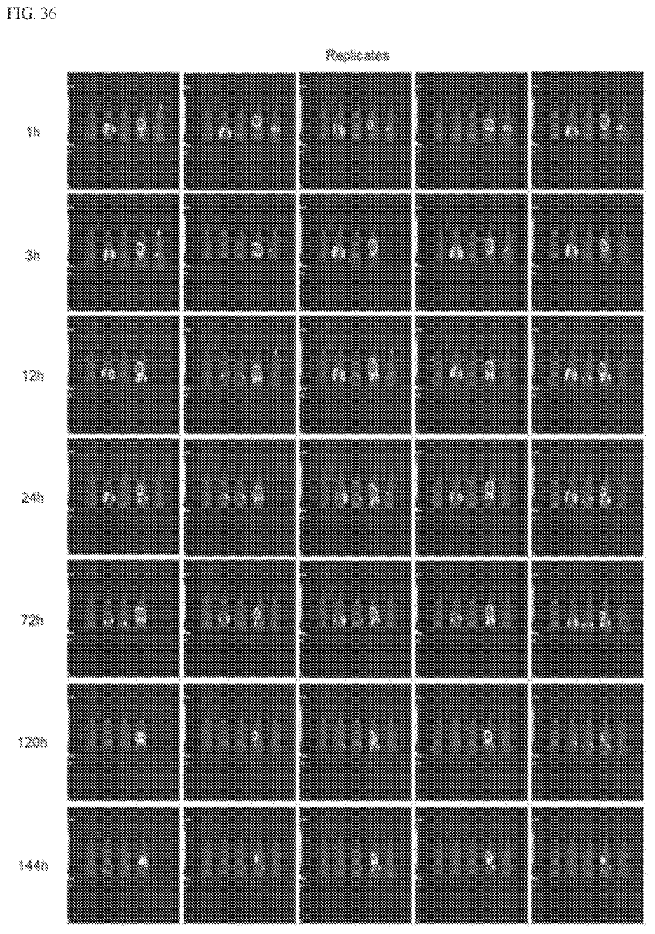

SHK-1 elite mice were dosed with DEDC(NP) or DEDC free dye in normal saline via intravenous, intraperitoneal, subcutaneous, or per os administration. Using whole-body live-animal fluorescence imaging renal fluorescence intensity increased in DEDC(NP) treated mice compared to DEDC free dye mice (FIG. 35a). Renal fluorescence intensity was measured 1, 3, 12, 24, 72, 120 and 144 h post administration (FIG. 35b). For all routes of administration, DEDC(NP) fluorescence intensity peaked between 12 and 24 h and steadily decreased over 144 h. DEDC(NP) intravenous administration resulted in greater renal fluorescence intensity at all time points up to ˜96 h. DEDC(NP) administered via intraperitoneal (i.p.) and subcutaneous (s.c.) injection also resulted in substantial renal accumulation with s.c. administration showing the second highest renal fluorescence intensity over 144 h. As expected per os administration resulted in minimal renal fluorescence. The fluorescence signal immediately following injection (FIG. 36) is likely the stomach and intestinal tract which is completely dissipated by 12 h. To examine the selectivity of intravenously administered DEDC(NP)s to the kidneys, additional mice dosed with 30 mg/kg DEDC(NP)s were euthanized 3 h post injection and their organs were harvested and imaged for fluorescence (FIG. 35c). At this time point renal fluorescence intensity was 3.6-fold greater than the next highest organ, the liver (FIG. 35d). Additionally, when compared to cardiac fluorescence, DEDC(NP)s showed 47-fold greater renal localization to the kidneys than the heart (FIG. 35e) demonstrating that nanoparticles significantly accumulate in the kidneys for a period of at least one week and avoid cardiac localization.

### Formoterol Containing Nanoparticles Increase Electron Transport Chain Protein Expression in the Renal Cortex

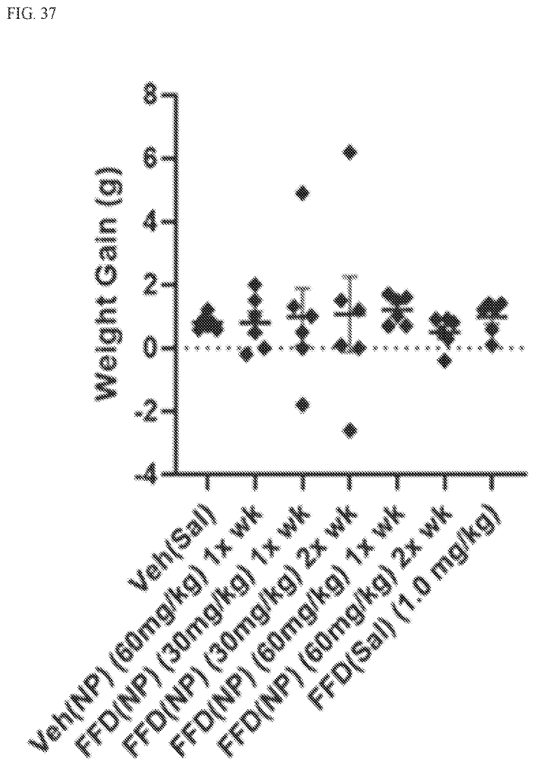

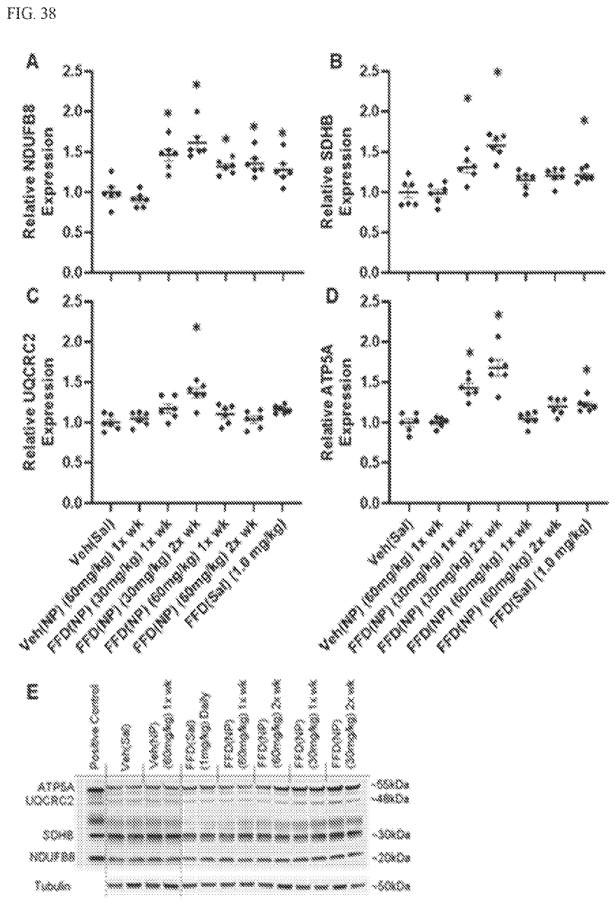

Given that FFD(NP)s demonstrate controlled release of formoterol over 144 h in vitro and that fluorescently labeled DEDC(NP)s show lasting accumulation in the kidneys of mice in vivo, the optimum FFD(NP) dosage and dosing frequency was determined. The FFD(Sal) dosage of 1 mg/kg administered daily was selected based on previously published work showing efficacy in a pre-diabetic mouse model (K. H. Cleveland, F. C. Brosius, and R. G. Schnellmann, “Regulation of mitochondrial dynamics and energetics in the diabetic renal proximal tubule by the β,” (in eng), Am J Physiol Renal Physiol, vol. 319, no. 5, pp. F773-F779, November 2020). Mice were administered FFD(NP) either once or twice per week at either 30 mg/kg or 60 mg/kg (Table 6) and compared to Veh(NP) 60 mg/kg twice per week, Veh(Sal) daily and FFD(Sal) 1 mg/kg daily. Mice were weighed at the conclusion of the study and showed no differences in weight gain amongst groups (FIG. 37). Mice administered FFD(NP)s at 30 mg/kg either once or twice per week showed increases in renal cortical electron transport chain (ETC) proteins NDUFB8 (1.4-1.6-fold), SDHB (1.2-1.5-fold), UQCRC2 (1.2-1.4-fold), and ATP5A (1.4-1.7-fold) (FIG. 38). Mice administered FFD(NP) at 60 mg/kg once and twice per week showed lower or no increases compared to the 30 mg/kg groups (FIG. 38). FFD(Sal) showed increased expression of NDUFB8 (1.3-fold), SDHB (1.3-fold) and ATP5A (1.2-fold) indicating that this strain of mice was similarly responsive to formoterol induced increases in ETC protein expression, which is an established indicator of mitochondrial biogenesis (MB). Additionally, mice exposed to Veh(NP)s at 60 mg/kg bi-weekly, the highest nanoparticle dose, showed no change in ETC protein expression (FIG. 38) indicating that the nanoparticles did not impact mitochondrial proteins in the renal cortex.

### Body Weight and Fasting Blood Glucose of Formoterol Containing Nanoparticle Treated Mice

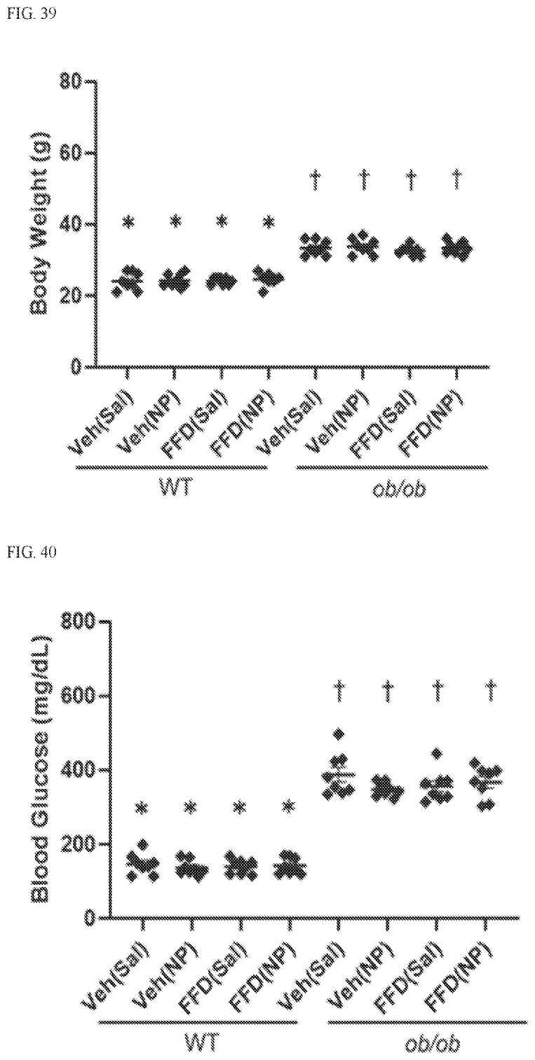

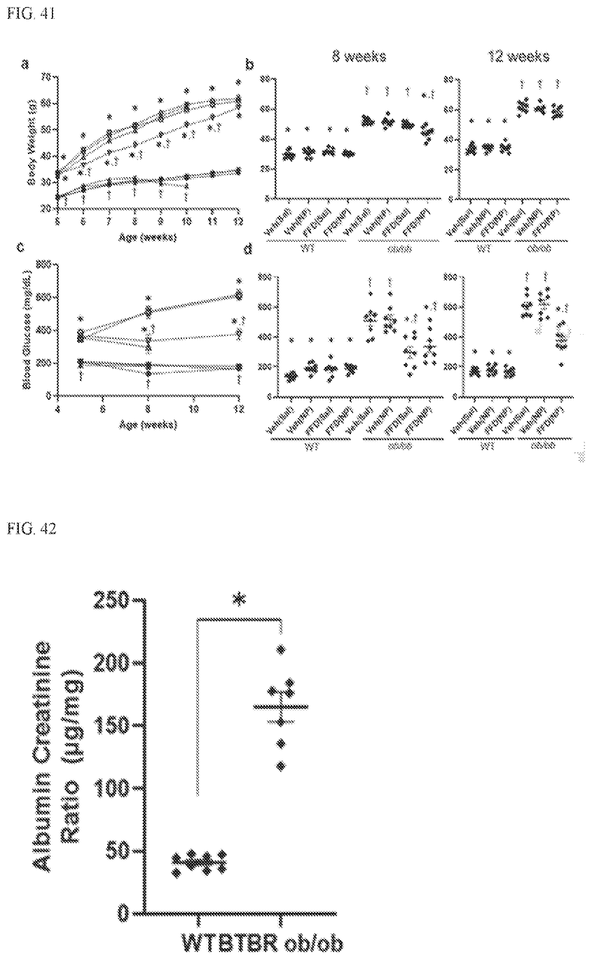

At 5 weeks of age, when the treatment was initiated, BTBR ob/ob mice already presented with elevated body weight (1.3-fold) and fasting blood glucose (2.6-fold) (FIG. 39) and fasting blood glucose (FIG. 40) compared to WT controls indicative of the diabetic phenotype. Both measures remained elevated for all BTBR ob/ob mice throughout the duration of the study (FIG. 41). FFD(NP) treated mice exhibited decreased weight gain between 6 and 11 weeks (FIG. 41a, b) compared to Veh(Sal) ob/ob mice. However, this was not seen with FFD(Sal) treated diabetic mice and the differences diminished by 12 weeks of age (FIG. 41b). Both FFD(Sal) and FFD(NP) treated obese diabetic groups exhibited lower fasted blood glucose levels at 8 and 12 weeks of age compared to Veh(Sal) and Veh(NP) treated diabetic mice. No differences were seen between the WT treated groups (FIG. 41d, e).

### Formoterol Containing Nanoparticles Ameliorated Polyuria, Albuminuria, Glomerular Hyperfiltration and Markers of Tubular Injury

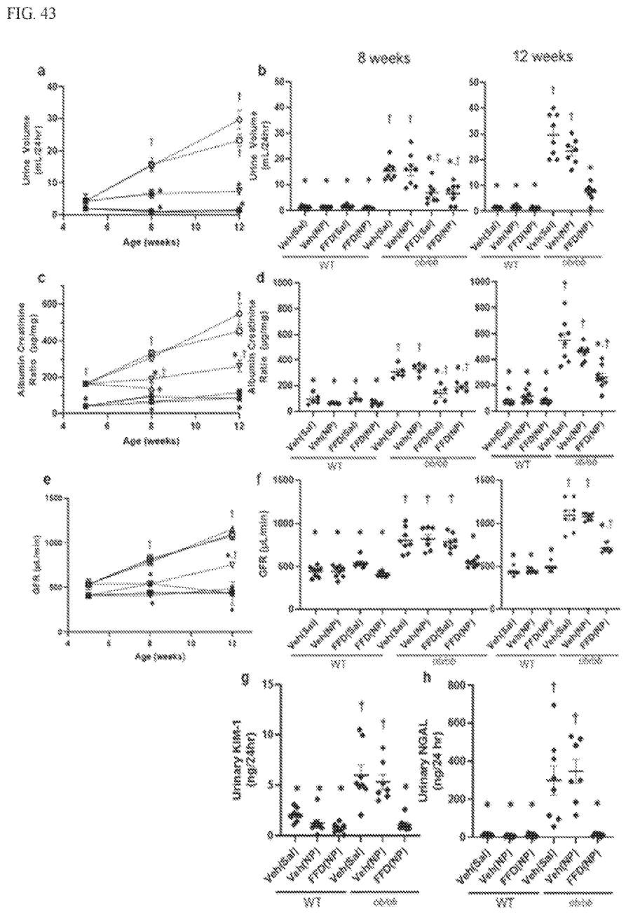

Veh(Sal) treated BTBR ob/ob mice have increased urine output at 8 weeks of age compared to their WT controls (FIG. 43a, b). Treatment with either FFD(Sal) or FFD(NP) resulted in a decrease in total urine output (47%) at 8 weeks. While the urine output of Veh(Sal) and Veh(NP) treated BTBR ob/ob mice increases throughout the study, formoterol treated mice show minimal (1.1-fold) increase in urine output over the course of the study. Further, vehicle treated BTBR ob/ob mice exhibit elevated albuminuria and albumin creatinine ratio (ACR) starting at 5 weeks of age (FIG. 42) and increasing (2.5-fold) at 12 weeks of age. FFD(Sal) and FFD(NP) treatment slowed the progression of ACR (FIG. 43c) and FFD(NP) treated mice had reduced (43%) ACRs by 12 weeks of age compared to vehicle controls (FIG. 43d).

Similarly, glomerular hyperfiltration which is common during the initial stages of DKD, is observed in vehicle treated BTBR ob/ob mice in which GFR is increased at 8 weeks of age (FIG. 43e, f). FFD(NP) treatment alone slows the progression of glomerular hyperfiltration. FFD(NP) treated BTBR ob/ob mice show no increase in GFR at 8 weeks of age and at 12 weeks of age demonstrate decreased hyperfiltration (39%) than BTBR ob/ob Veh(Sal) treated mice (FIG. 43f). Urinary KIM-1 and NGAL, both markers of renal injury, are elevated in the BTBR ob/ob vehicle treated mice compared to WT controls (FIG. 43g, h). This is not the case in FFD(NP) treated BTBR ob/ob mice which are indistinguishable from BTBR WT controls. This further supports that FFD(NP) treatment is protecting from sub-acute/chronic diabetic kidney injury.

### Formoterol Containing Nanoparticles Reduce Glomerular Injury in Diabetic Mice

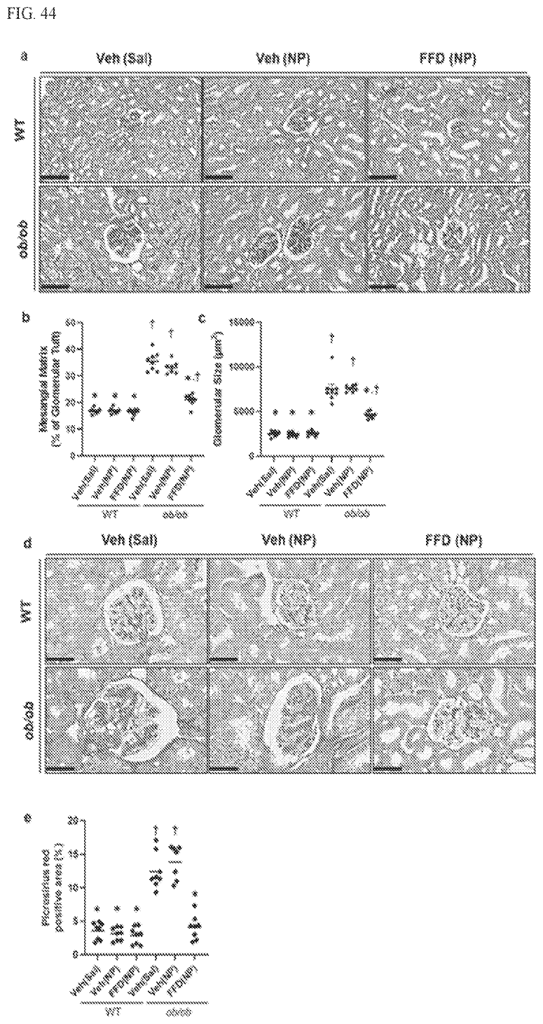

Veh(Sal) treated BTBR ob/ob mice showed significant mesangial matrix expansion (1.9-fold) (FIG. 44a, b) and increased glomerular size (2.7-fold) (FIG. 44a, c) compared to BTBR WT Veh(Sal) control. Mesangial matrix expansion was reduced with FFD(NP) treatment at 12 weeks of age, with FFD(NP) treated BTBR ob/ob mice having only 1.1-fold greater mesangial matrix than BTBR WT controls. Additionally, FFD(NP) treated BTBR ob/ob mice showed less glomerular hypertrophy than Veh(Sal) and Veh(NP) BTBR ob/ob mice (36% decrease). In addition, glomerulosclerosis as measured by picrosirius red staining was increased in BTBR ob/ob Veh(Sal) treated mice (3.4-fold increase) (FIG. 44d, e). Comparatively, FFD(NP) treated BTBR ob/ob mice showed reduced glomerular accumulation of collagen I and III compared to BTBR ob/ob Veh(Sal) treated mice (67% decrease). Arteriolar hyalinosis was not detected in any of the BTBR ob/ob or BTBR WT groups. This confirms that BTBR ob/ob vehicle mice develop significant glomerular injury and demonstrates a reduction in injury with FFD(NP) treatment.

### Formoterol Containing Nanoparticles Mitigates Renal Tubulointerstitial Damage.

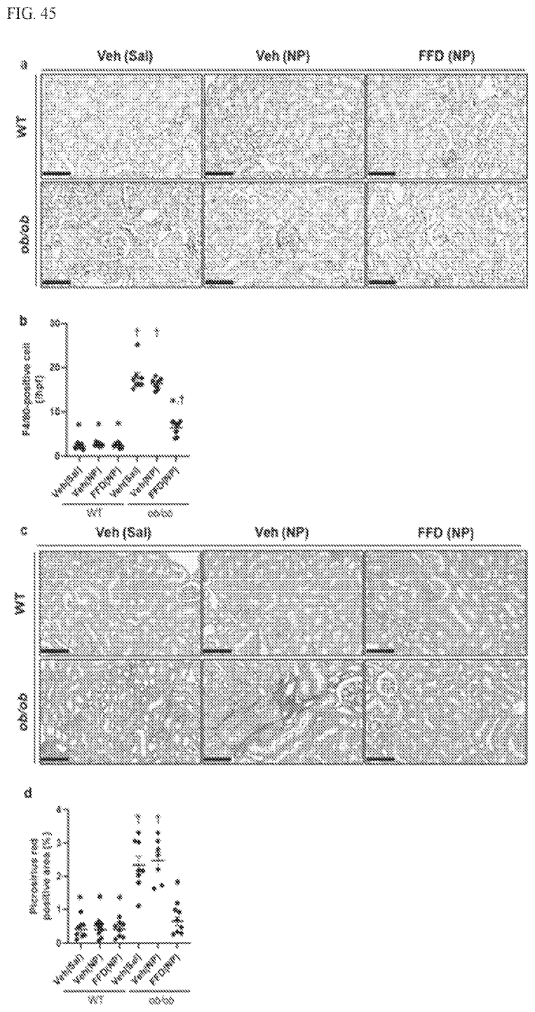

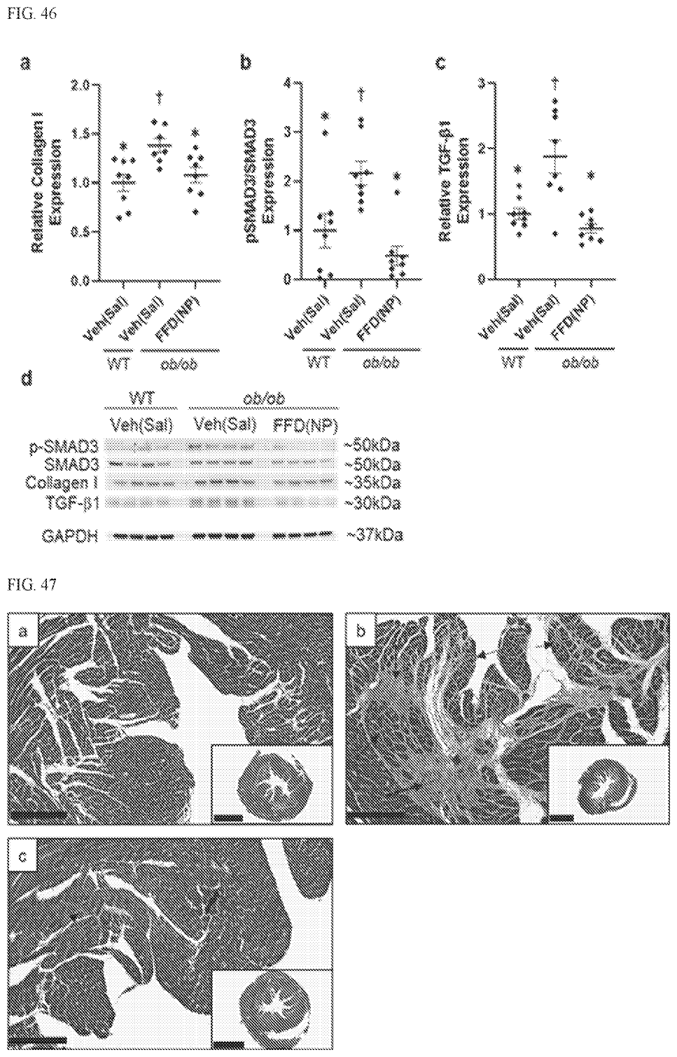

Tubulointerstitial inflammation, characterized by macrophage infiltration, is an important driver of tubulointerstitial fibrosis. The number of infiltrating macrophages, as measured by F4/80-positive cells per field in the renal cortex, was significantly elevated (18-fold) in BTBR ob/ob Veh(Sal) treated mice (FIG. 45a). FFD(NP) treatment reduced the number of infiltrating macrophages (61%) (FIG. 45a, b). There was no evidence of elevated inflammatory response in BTBR WT groups. Only one of the Veh(Sal) treated ob/ob mice showed any sign of glomerular infiltration of macrophages. In line with tubulointerstitial inflammation, tubulointerstitial fibrosis as measured by picrosirius red staining revealed diffuse fibrosis in BTBR ob/ob Veh(Sal) treated mice (FIG. 45c) that was 5.2-fold greater than WT controls. Fibrosis was improved with FFD(NP) treatment (62% reduction) compared to BTBR ob/ob Veh(Sal) control (FIG. 45c, d). This is further substantiated by total cortical collagen I expression levels (FIG. 46a) which show a 1.4 fold increase in renal cortical expression in Veh(Sal) treated BTBR ob/ob mice and no significant increase in FFD(NP) treated BTBR ob/ob mice. Additionally, BTBR ob/ob mice saw an increased ratio of phosphorylated:total SMAD3 (FIG. 46b) as well as increase TGF-β1 expression (FIG. 46c) which are central in the TGF-β-mediated signaling pathway in the pathogenesis of fibrosis. Treatment of BTBR ob/ob mice with FFD(NP) results in a decrease in collagen I expression (30%) compared to BTBR ob/ob Veh(Sal) controls and no difference compared to BTBR WT Veh(Sal) controls. FFD(NP) treatment also results in reduced TGF-β1 expression (44%) and SMAD3 phosphorylation (77%) compared to Veh(Sal) treated BTBR ob/ob mice.

### Formoterol Containing Nanoparticles Protect Against Formoterol-Induced Cardiovascular Toxicity

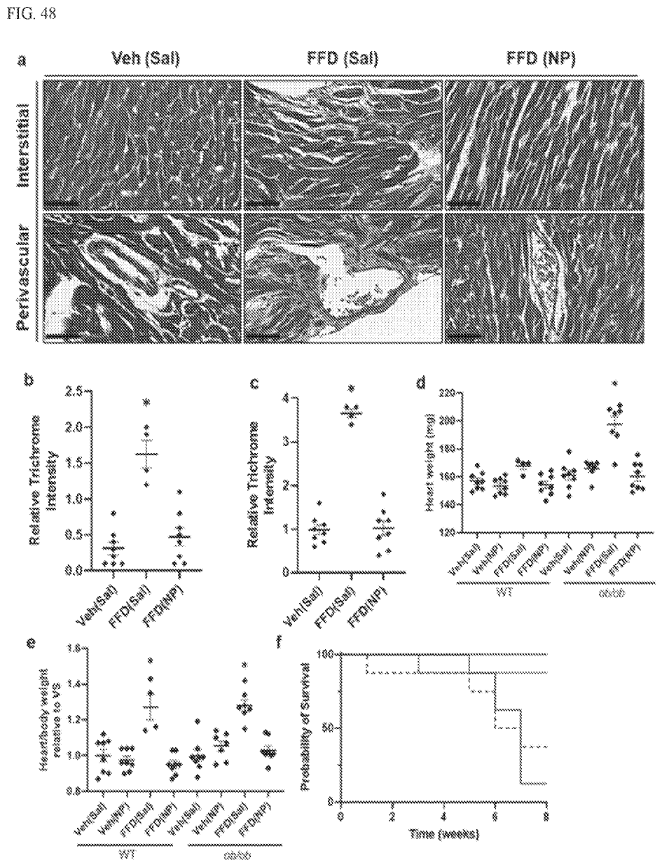

Previous research has shown that formoterol and similar beta-agonists have the potential for cardiovascular toxicity. Recently we have shown that 0.3 mg/kg of formoterol given intraperitoneally daily for one week to C57Bl/6NCrl mice results in profound tachycardia and diastolic dysfunction as well as cardiac hypertrophy. That research also demonstrated that renally targeted polymeric nanoparticles similar to the ones employed in this study did not show signs of acute cardiotoxicity. Here, FFD(Sal) treated mice showed significant focal (FIG. 47b) and diffuse (FIG. 48a) collagen accumulation in the left ventricular wall of the heart. Diffuse interstitial trichrome positive area was elevated 1.6-fold with FFD(Sal) treatment (FIG. 48b) and perivascular trichrome positive area was elevated 3.7-fold (FIG. 48c). Notably, there was no increase in trichrome staining intensity in either interstitial or perivascular regions in FFD(NP) treated mice and there was no evidence of focal myocardial fibrosis (FIG. 47a, c). Formoterol treatment of both diabetic and WT mice resulted in cardiac hypertrophy. FFD(Sal) treated BTBR ob/ob mice showed an increase in raw cardiac weight (FIG. 48d) which was 1.3-fold increase over Veh(Sal) treated BTBR ob/ob mice. When normalized to their relative Veh(Sal) controls, either BTBR WT or BTBR ob/ob, FFD(Sal) treated mice showed comparable 1.3-fold increases over their respective vehicle controls (FIG. 48e). Neither FFD(NP) treated group showed any increase in heart weight relative to controls. The hypertrophic cardiomyopathy observed in FFD(Sal) treated mice is potentially a driver for decreased survival rate of FFD(Sal) treated mice in this study. BTBR ob/ob mice treated with FFD(Sal) had only a 13% probability of surviving to 12 weeks of age and BTBR WT mice treated with FFD(Sal) had a 38% probability of survival over the same time range compared to an 88% survival probability for the next nearest group (Veh(NP) treated BTBR ob/ob mice) (FIG. 48f).

All publications and patents mentioned in the present application are herein incorporated by reference. Various modification and variation of the described methods and compositions of the disclosure will be apparent to those skilled in the art without departing from the scope and spirit of the disclosure. Although the disclosure has been described in connection with specific preferred embodiments, it should be understood that the disclosure as claimed should not be unduly limited to such specific embodiments. Indeed, various modifications of the described modes for carrying out the disclosure that are obvious to those skilled in the relevant fields are intended to be within the scope of the following claims.

1. A composition, comprising:
a pharmaceutical composition comprising nanoparticles comprising a β₂ adrenergic agonist and a polymer.

2. The composition of claim 1, wherein said β₂ adrenergic agonist is interspersed within said polymer.

3. The composition of claim 1, wherein said β₂ adrenergic agonist is selected from the group consisting of albuterol, formoterol, bitolterol, fenoterol, isoproterenol, levalbuterol, metaproterenol, pirbuterol, procaterol, ritodrine, terbutaline, arformoterol, bambuterol, clenbuterol, salmeterol, abediterol, carmoterol, indacaterol, olodaterol, vilanterol, isoxsuprine, mabuterol, and zilpaterol.

4. The composition of claim 1, wherein said polymer is biocompatible and biodegradable.

5. The composition of claim 4, wherein said polymer is selected from the group consisting of methyl ether-block-poly(lactide-co-glycolide) (PLGA), poly(vinyl alcohol), chitosan, poly(ε-caprolactone), poly(ethylene glycol) methyl ether-block-poly(lactide-co-glycolide) (PLGA-PEG), Poly(lactide-co-glycolide) methyl ether block-poly(ethylene glycol)-amine (PLGA-PEG-HN2), and poly(lactide-co-glycolide) methyl ether block-poly(ethylene glycol)-carboxylic acid (PLGA-PEG-COOH).

6. The composition of claim 5, wherein said PLGA has an average molecular weight of 10,000 to 100,000 and said PEG has an average molecular weight of 2,000 to 10,000.

7. The composition of claim 6, wherein said PLGA has an average molecular weight of 55,000 and said PEG has an average molecular weight of 5,000.

8. The composition of claim 1, wherein said particles are spherical.

9. The composition of claim 1, wherein said nanoparticles have a diameter of 100 to 800 nm.

10. The composition of claim 1, wherein said β₂ adrenergic agonist is released from said nanoparticle at physiological conditions.

11. The composition of claim 1, wherein each of said nanoparticles comprises 1 to 5 μg of said β₂ adrenergic agonist per mg of nanoparticles.

12. The composition of claim 1, wherein said composition further comprises a pharmaceutically acceptable carrier.

13. (canceled)

14. The composition of claim 1, wherein said nanoparticles are made by a method, comprising:
a) mixing a first solution comprising said β₂ adrenergic agonist with a second solution comprising an emulsion of said polymer in a solvent;
b) emulsifying said first solution in said second solution; and
c) removing said solvent.

15. The composition of claim 1, wherein said nanoparticles are made by a method, comprising:
a) mixing a first solution comprising said β₂ adrenergic agonist with a second solution comprising an emulsion of said polymer in a solvent;
b) emulsifying said first solution in said second solution to generate a first emulsion;
c) mixing and emulsifying said first emulsion with a third solution comprising said polymer; and
d) removing said solvent.

16. A method of treating or preventing kidney disease, comprising:
administering the composition of claim 1 to a subject in need thereof.

17. The method of claim 16, wherein said administering is intravenous, intraperitoneal, or subcutaneous administration.

18. The method of claim 16, wherein said nanoparticles localize to the kidney of said subject.

19. The method of claim 18, wherein said nanoparticles localize to the tubules of the renal cortex.

20-21. (canceled)

22. The method of claim 16, wherein said administration results in mitochondrial biogenesis in the renal proximal tubules.

23. The method of claim 16, wherein said kidney disease is selected from the group consisting of acute renal injury, chronic renal injury, glomerular injury, drug and toxicant induced renal injury, ischemia-reperfusion injury, and diabetic nephropathy

24-26. (canceled)
